(19) United States
(12) Patent Application Publication
(10) Pub. No.: US 2007/0161578 A1
(43) Pub. Date: Jul. 12, 2007

(54) Treatment of nonalcoholic fatty liver disease using cholesterol lowering agents and/or H3 receptor antagonist/inverse agonist

(76) Inventors: Joyce J. Hwa, Westfield, NJ (US); Margaret van Heek, Scotch Plains, NJ (US); Harry Davis JR., Berkeley Heights, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/641,527

(22) Filed: Dec. 19, 2006

Related U.S. Application Data
(60) Provisional application No. 60/855,178, filed on Oct. 30, 2006.
(60) Provisional application No. 60/836,642, filed on Aug. 9, 2006.
(60) Provisional application No. 60/787,048, filed on Mar. 29, 2006.
(60) Provisional application No. 60/752,710, filed on Dec. 21, 2005.

(51) Int. Cl. A61K 31/397; A61K 31/7052

(52) U.S. Cl. 514/23; 514/210.02

(57) ABSTRACT

This invention provides for method for the treatment, prevention or ameliorating the symptoms of nonalcoholic fatty liver disease (NAFLD) in a mammal in need thereof comprising the step of administering an effective amount of a composition comprising a therapeutic combination of at least one cholesterol lowering agent and/or at least one H₃ antagonist/inverse agonist.

### RELATED APPLICATION

This application claims the benefit to provisional application U.S. Ser. No. 60/855,178 filed Oct. 30, 2006, provisional application U.S. Ser. No. 60/752,710, filed Dec. 21, 2005, provisional application U.S. Ser. No. 60/787,048, filed Mar. 29, 2006, and provisional application U.S. Ser. No. 60/836,642, filed Aug. 9, 2006, all herein incorporated by reference.

### FIELD OF THE INVENTION

The present invention relates to a method for treating nonalcoholic fatty liver disease in a mammal by administering an effective amount of therapeutic composition comprising at least one cholesterol lowering agent and/or at least one H₃ receptor antagonist/inverse agonist.

### BACKGROUND OF THE INVENTION

Nonalcoholic fatty liver disease (NAFLD) describes a spectrum of liver diseases ranging from simple fatty liver (steatosis) to nonalcoholic steatohepatitis (NASH) with progressive fibrosis and liver failure. Hyperglycemia with or without evidence of hyperlipidemia is commonly associated with NAFLD. The disease exhibits the histological features of alcohol-induced liver disease in patients who do not consume significant amounts of alcohol. All of the stages of NAFLD have in common the accumulation of fat in the liver cells. Farrell and Larter in Hepatology, 243:S99-S112 (2006) describe NASH as “the lynchpin” between hepatic steatosis and cirrhosis in the spectrum of NAFLD. See also, Palekar, et al., Liver Int., 26(2):151-6 (2006). In NASH, the fat accumulation of associated with varying degrees of inflammation and fibrosis. Conditions most commonly associated with NAFLD are obesity, type 2 diabetes and metabolic syndrome.

US Publication No. 2004/29805 describes a method for preventing or treating NAFLD by administering an agent that antagonizes the receptor to glucose-dependent insulinotropic polypeptide. Yamagishi et al. advance a hypothesis that ezetimibe might be a new therapeutic approach for the treatment of NAFLD (Medical Hypotheses, 66, pp. 844-846 (2006)) (available on line in September 2005).

Treatments for NASH include diet and exercise and/or administering probucol, clofribrate, gemfibrozil, betaine, vitamins E and/or C, metformin, toglitaxone, rosiglitazone or plogitazone and vitamin E. M. Charlton, Clinical Gastroenterology and Hepatology, 2(12), 1048-56 (2004); P. Portincaso et al., Clinical Biochemistry, 38, 203-17 (2005). US Publication No. 2004/105870A1 describes a treatment for NASH which comprises administering a formulation comprising dietary lecithin supplement, vitamin B complex or an antioxidant. US Publication Nos. 2005/0032823A1 and 2004/0102466A1 describe pyrimidine derivatives, which are selective COX-2 inhibitors, as useful in treating NASH. Other compounds for the treatment of fatty liver disease are described in US Publication No. 2005/0004115A1. There is no mention of cholesterol absorption inhibitors or H₃ receptor antagonists/inverse agonist as being useful in treating NAFLD or NASH.

Beltroy et al. (Abstract, American College of Gastroenterology Meeting, 2004) discuss the effect of ezetimibe treatment on Niemann-Pick type C mice. These mice have elevated liver enzymes (ACT and AST) and steatosis and, therefore, have steato hepatitis. Beltroy et al. indicate that ezetimibe treatment reduced hepatic cholesterol accumulation and improved histological abnormalities and liver enzymes.

Compounds that inhibit cholesterol absorption in the small intestine are well known in the art and are described, for example, in U.S. Re. Pat. No. 37,721; U.S. Pat. No. 5,631,356; U.S. Pat. No. 5,767,115; U.S. Pat. No. 5,846,966; U.S. Pat. No. 5,698,548; U.S. Pat. No. 5,633,246; U.S. Pat. No. 5,656,624; U.S. Pat. No. 5,624,920; U.S. Pat. No. 5,688,787; U.S. Pat. No. 5,756,470; US Publication No. 2002/0137689; WO 02/066464; WO 95/08522 and WO96/19450. Each of the aforementioned publications is incorporated by reference. The art indicates that these compounds are useful in treating, for example, atherosclerotic coronary disease, either by administrating these compounds alone or with a second compound such as a cholesterol biosynthesis inhibitor. These documents do not indicate that these inhibitors are useful in treating NAFLD.

U.S. Pat. Nos. 5,846,966 and 5,661,145, respectively, disclose treatments for inhibiting atherosclerosis and reducing plasma cholesterol levels using such hydroxy-substituted azetidinone compounds or substituted β-lactam compounds in combination with HMG-CoA reductase inhibitor compounds, which act by blocking hydroxymethylglutaryl coenzyme A (HMG-CoA) reductase (the rate-limiting enzyme in hepatic cholesterol synthesis). HMG-CoA reductase inhibitors, e.g., statins such as lovastatin, simvastatin, and pravastatin, slow the progression of atherosclerotic lesions in the coronary and carotid arteries. Simvastatin and pravastatin have also been shown to reduce the risk of coronary heart disease events in patients with hypercholesterolemia and/or atherosclerotic coronary heart disease (CHD).

Simvastatin is marketed worldwide, and sold in the U.S. under the tradename ZOCOR®. Methods for making it are described in U.S. Pat. Nos. 4,444,784; 4,916,239; 4,820,850; among other patent and literature publications.

H₃ receptor antagonists/inverse agonists are well known in the art. H₃ receptor sites are found on sympathetic nerves, where they modulate sympathetic neurotransmission and attenuate a variety of end organ responses under control of the sympathetic nervous system. Specifically, H₃ receptor activation by histamine attenuates norepinephrine outflow to resistance and capacitance vessels, causing vasodilation. H₃ receptor antagonists/inverse agonists are known to treat: allergy, allergy-induced airway (e.g., upper airway) responses, congestion (e.g., nasal congestion), hypotension, cardiovascular disease, diseases of the GI tract, hyper and hypo motility and acidic secretion of the gastro-intestinal tract, obesity, sleeping disorders (e.g., hypersomnia, somnolence, and narcolepsy), disturbances of the central nervous system, attention deficit hyperactivity disorder (ADHD), hypo and hyperactivity of the central nervous system (for example, agitation and depression), and/or other CNS disorders (such as Alzheimer's, schizophrenia, and migraine) in a patient such as a mammal. These compounds are particularly useful for treating allergy, allergy-induced airway responses and/or congestion.

WO 95/14007 published May 26, 1995 and incorporated by reference discloses H₃ receptor antagonists of the imidazole type.

WO99/24405 and incorporated by reference published May 20, 1999 discloses H₃ receptor ligands of the imidazole type.

U.S. Pat. No. 6,720,328 B1, issued on Apr. 13, 2004 and incorporated by reference, discloses non-imidazole H₃ receptor antagonists. U.S. Publication US 2004/0019099, published on Jan. 29, 2004 and incorporated by reference, discloses indole derivatives that are H₃ receptor antagonists. U.S. Publication US 2004/0048843A1, published on Mar. 11, 2004 and incorporated by reference, and U.S. Publication US 2004/0097483A1, published on May 20, 2004 and incorporated by reference, disclose benzimidazole derivatives as H₃ antagonists. Piperidine compounds that are H₃ antagonists are disclosed in U.S. Pat. No. 6,849,621; this document issued on Feb. 1, 2005 and is incorporated by reference.

WO 2004/110375 describes a combination therapy for the treatment of diabetes wherein the combination comprises an anti-obesity agent, such as an H₃ receptor antagonist/inverse agonist and an anti-diabetic agent. The publication indicates that other pharmaceutical agents including anti-dislipidemic agents, such as bile acid sequestrants and cholesterol absorption inhibitors, such as azetidinones, may be included.

U.S. Pat. No. 5,869,479 discloses compositions for the treatment of the symptoms of allergic rhinitis using a combination of at least one histamine HI receptor antagonist and at least one histamine H₃ receptor antagonist.

WO 2004/110368 describes combination therapies for the treatment of hypertension comprising the combination of an anti-obesity agent and an anti-hypertensive agent.

WO 2005/000217 describes combination therapies for the treatment of dyslipidemia comprising the administration of a combination of an anti-obesity agent and an anti-dyslipidemic agent.

WO 2004/110375 describes combination therapies for the treatment of diabetes comprising the administration of a combination of an anti-obesity agent and an anti-diabetic agent.

US 2004/0122033 describes combination therapies for the treatment of obesity comprising the administration of a combination of an appetite suppressant and/or metabolic rate enhancers and/or nutrient absorption inhibitors. US 2004/0229844 describes combination therapies for treating atherosclerosis comprising the administration of a combination of nicotinic acid or another nicotinic acid receptor agonist and a DP receptor antagonist.

U.S. Pat. Nos. 6,437,147, 6,756,384, and 2003/0135056 describe combinations of imidazo heterocyclic compounds which bind to the H₃ receptor with antiobesity agents or appetite regulating agents, including sibutramine, phentermine, topiramate, lovastatin, pravastatin, and simvastatin.

### SUMMARY OF THE INVENTION

The present invention provides for a method for the treatment, prevention or ameliorating the symptoms of nonalcoholic fatty liver disease (NAFLD) in a mammal in need thereof by administering an effective amount of a composition comprising at least one cholesterol lowering agent, e.g., a sterol absorption inhibitor, a 5-α-stanol absorption inhibitor or a HMG-CoA reductase inhibitor and/or at least one H₃ antagonist/inverse agonist.

An alternative embodiment of this invention provide for the prevention or amelioration the symptoms or development of hepatic steatosis in a mammal in need thereof by administering at least one cholesterol lowering agent, e.g., a sterol absorption inhibitor, a 5-α-stanol absorption inhibitor or a HMG-CoA reductase inhibitor and/or at least one H₃ receptor antagonists/inverse agonist.

Another embodiment of this invention also provides for the prevention or amelioration of the development of nonalcoholic steatohepatitis (NASH) in a mammal by administering an effective amount of a therapeutic combination comprising at least one cholesterol lowering agent, e.g., a sterol absorption inhibitor, a 5-α-stanol absorption inhibitor or an HMG-CoA reductase inhibitor and/or at least one H₃ receptor antagonist/inverse agonist.

A further embodiment of this invention provides for the prevention or amelioration of the development of cirrhosis and heptacellular carcinoma in a mammal by administering an effective amount of a therapeutic combination comprising at least one cholesterol lowering agent, e.g., a sterol absorption inhibitor, a 5-α-stanol absorption inhibitor or an HMG-CoA reductase inhibitor and/or at least one H₃ receptor antagonist/inverse agonist to said mammal.

Another embodiment of this invention provides for a method for the treatment, prevention or ameliorating the symptoms of NAFLD or NASH in a mammal in need thereof by administering an effective amount of a composition comprising, in addition to at least cholesterol lowering agent, e.g., a sterol absorption inhibitor, a 5-α-stanol absorption inhibitor or an HMG-CoA reductase inhibitor, and/or at least one H₃ antagonist/inverse agonist, an antiobesity agent.

The present invention also relates to a kit for the treatment, prevention or amelioration of the symptoms of NAFLD which comprises at least one cholesterol lowering agent and/or at least one H₃ receptor/inverse agonist in separate form.

### BRIEF DESCRIPTION OF THE DRAWINGS

FIG. 1 depicts the effect of ezetimibe and the H₃ receptor antagonist/inverse agonist of Formula XIIIA on liver to body weight ratio in mice.

FIG. 2 depicts the effect of ezetimibe and the H₃ receptor antagonist/inverse agonist of Formula XIIIA on the levels of liver triglycerides in mice.

FIG. 3 depicts the effect of ezetimibe and the H₃ receptor antagonist/inverse agonist of Formula XIIIA on the levels of cholesterol ester in mice.

FIG. 4 depicts the effect of ezetimibe and the H₃ receptor antagonist/inverse agonist of Formula XIIIA on the levels of free cholesteryl in mice.

FIG. 5 depicts the effect of ezetimibe and the H₃ receptor antagonist/inverse agonist of Formula XIIID on plasma alanine aminotransferase (ALT) enzyme activities in mice.

FIG. 6 depicts the effect of ezetimibe on liver to body weight ratio in mice.

FIG. 7 depicts the effect of ezetimibe on the levels of liver triglycerides in mice.

FIG. 8 depicts the effect of ezetimibe on the levels of cholesteryl ester in mice.

FIG. 9 depicts the effect of ezetimibe on the levels of free cholesterol in mice.

### DETAILED DESCRIPTION

The terms used herein have their ordinary meaning and the meaning of such terms is independent at each occurrence thereof. That notwithstanding and except where stated otherwise, the following definitions apply throughout the specification and claims: Chemical names, common names and chemical structures may be used interchangeably to describe that same structure. These definitions apply regardless of whether a term is used by itself or in combination with other terms, unless otherwise indicated. Hence, the definition of “alkyl” applies to “alkyl” as well as the “alkyl” protion of “hydroxyalkyl”, “haloalkyl”, “alkoxy” etc.

As used above, and throughout the specification, the following terms, unless otherwise indicated, shall be understood to have the following meanings:

“Patient” includes both human and animals.

“Mammal” means humans and other mammalian animals.

“Alkyl” means an aliphatic hydrocarbon group which may be straight or branched and comprising about 1 to about 20 carbon atoms in the chain which may be optionally substituted with groups, such as, for example, hydroxyl, cyano, halo, alkoxy, aryloxy, heteroaryl heteroxy, —C(O)OH, —C(O)Oalkyl, N₃, amino, dialkylamino, alkylamino, NO₂ mercapto, alkylthio, cycloalkyl and the like. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. Non-limiting examples of suitable alkyl groups include methyl, ethyl, n-propyl, isopropyl, n-butyl, t-butyl, n-pentyl, heptyl, nonyl and decyl. Non-limiting examples of suitable substituted alkyl groups include fluoromethyl, trifluoromethyl and cyclopropylmethyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. Non-limiting examples of suitable alkenyl groups include ethenyl, propenyl, n-butenyl, 3-methylbut-2-enyl, n-pentenyl, octenyl and decenyl.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl, 3-methylbutynyl, n-pentynyl, and decynyl.

“Aryl” means an aromatic monocyclic or multicyclic ring system comprising about 6 to about 14 carbon atoms, preferably about 6 to about 10 carbon atoms. The aryl group can be optionally substituted with one or more substituents, which may be the same or different, and are as defined herein or two substituents on adjacent carbons can be linked together to form 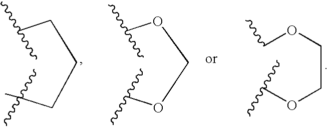
Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one to four of the ring atoms is an element other than carbon, for example nitrogen, oxygen or sulfur, alone or in combination. Preferred heteroaryls contain about 5 to about 6 ring atoms. The “heteroaryl” can be optionally substituted by one or more substituents, which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more substituents which may be the same or different, and are as defined herein. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalin, norbornyl, adamantyl and the like. Further non-limiting examples of cycloalkyl include the following:

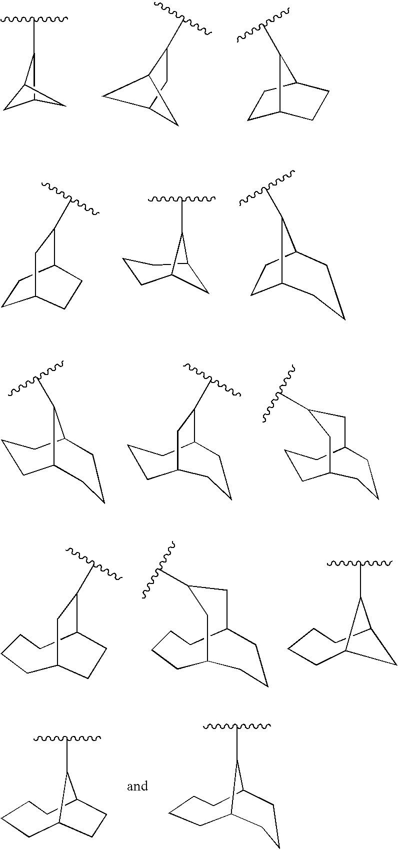

“Cycloalkylether” means a non-aromatic ring of 3 to 7 members comprising an oxygen atom and 2 to 7 carbon atoms. Ring carbon atoms can be substituted, provided that substituents adjacent to the ring oxygen do not include halo or substituents joined to the ring through an oxygen, nitrogen or sulfur atom.

“Cycloalkenyl” means a non-aromatic mono or multicyclic ring system comprising about 3 to about 10 carbon atoms, preferably about 5 to about 10 carbon atoms which contains at least one carbon-carbon double bond. The cycloalkenyl ring can be optionally substituted with one or more substituents which may be the same or different, and are as defined herein. Preferred cycloalkenyl rings contain about 5 to about 7 ring atoms. Non-limiting examples of suitable monocyclic cycloalkenyls include cyclopentenyl, cyclohexenyl, cycloheptenyl, and the like. Non-limiting example of a suitable multicyclic cycloalkenyl is norbornylenyl.

“Heterocyclenyl” (or “heterocycloalkeneyl”) means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more substituents. The nitrogen or sulfur atom of the heterocyclenyl can be optionally oxidized to the corresponding N-oxide, S-oxide or S,S-dioxide. Non-limiting examples of suitable monocyclic azaheterocyclenyl groups include 1,2,3,4-tetrahydropyridyl, 1,2-dihydropyridyl, 1,4-dihydropyridyl, 1,2,3,6-tetrahydropyridyl, 1,4,5,6-tetrahydropyrimidyl, 2-pyrrolinyl, 3-pyrrolinyl, 2-imidazolinyl, 2-pyrazolinyl, and the like. Non-limiting examples of suitable oxaheterocyclenyl groups include 3,4-dihydro-2H-pyran, dihydrofuranyl, fluorodihydrofuranyl, and the like. Non-limiting example of a suitable multicyclic oxaheterocyclenyl group is 7-oxabicyclo[2.2.1]heptenyl. Non-limiting examples of suitable monocyclic thiaheterocyclenyl rings include dihydrothiophenyl, dihydrothiopyranyl, and the like.

“Halo” means fluoro, chloro, bromo, or iodo groups. Preferred are fluoro, chloro or bromo, and more preferred are fluoro and chloro.

“Haloalkyl” means an alkyl as defined above wherein one or more hydrogen atoms on the alkyl is replaced by a halo group defined above.

“Heterocyclyl” (or heterocycloalkyl) means a non-aromatic saturated monocyclic or multicyclic ring system comprising about 3 to about 10 ring atoms, preferably about 5 to about 10 ring atoms, in which 1-3, preferably 1 or 2 of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. The heterocyclyl can be optionally substituted by one or more which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like.

“Arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Arylcycloalkyl” means a group derived from a fused aryl and cycloalkyl as defined herein. Preferred arylcycloalkyls are those wherein aryl is phenyl and cycloalkyl consists of about 5 to about 6 ring atoms. The arylcycloalkyl can be optionally substituted by one or more substituents. Non-limiting examples of suitable arylcycloalkyls include indanyl and 1,2,3,4-tetrahydronaphthyl and the like. The bond to the parent moiety is through a non-aromatic carbon atom.

“Arylheterocycloalkyl” means a group derived from a fused aryl and heterocycloalkyl as defined herein. Preferred arylcycloalkyls are those wherein aryl is phenyl and heterocycloalkyl consists of about 5 to about 6 ring atoms. The arylheterocycloalkyl can be optionally substituted by one or more substituents. Non-limiting examples of suitable arylheterocycloalkyls include

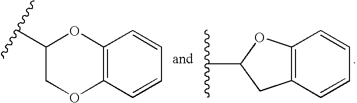

The bond to the parent moiety is through a non-aromatic carbon atom.

“Acyl” means an organic group in which the —OH of the carboxyl group is replaced by some other substituent. Suitable non-limiting examples include H—C(O)—, alkyl-C(O)—, alkenyl-C(O)—, alkynyl-C(O)—, aryl-C(O)— or cycloalkyl-C(O)— group in which the various groups are as previously described. The bond to the parent moiety is through the carbonyl. Preferred acyls contain a lower alkyl. Non-limiting examples of suitable acyl groups include formyl, acetyl, propanoyl, 2-methylpropanoyl, butanoyl and cyclohexanoyl.

“Alkoxy” means an alkyl-O— group in which the alkyl group is as previously described. Non-limiting examples of suitable alkoxy groups include methoxy, ethoxy, n-propoxy, isopropoxy, n-butoxy and heptoxy. The bond to the parent moiety is through the ether oxygen.

“Alkyoxyalkyl” means a group derived from an alkoxy and alkyl as defined herein. The bond to the parent moiety is through the alkyl.

“Arylalkenyl” means a group derived from an aryl and alkenyl as defined herein. Preferred arylalkenyls are those wherein aryl is phenyl and the alkenyl consists of about 3 to about 6 atoms. The arylalkenyl can be optionally substituted by one or more substituents. The bond to the parent moiety is through a non-aromatic carbon atom.

“Arylalkynyl” means a group derived from a aryl and alkenyl as defined herein. Preferred arylalkynyls are those wherein aryl is phenyl and the alkynyl consists of about 3 to about 6 atoms. The arylalkynyl can be optionally substituted by one or more substituents. The bond to the parent moiety is through a non-aromatic carbon atom.

The suffix “ene” on alkyl, aryl, hetercycloalkyl, etc. indicates a divalent moiety, e.g., —CH₂CH₂— is ethylene, and 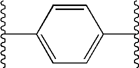
is para-phenylene.

The term “optionally substituted” means optional substitution with the specified groups, radicals or moieties, in available position or positions.

Substituents (“ring substituents”) for the aryl, heteroayl, cycloalkyl, cycloalkylether, cycloalkenyl, heterocyclenyl, heterocyclyl, arylcycloalkyl, arylheterocycloalkyl, arylalkenyl and arylalkynyl groups described above, include, for example, alkyl, cycloalkyl, aryl, heteroaryl, aryloxy, heteroaryloxy, cycloalkylether, cycloalkenyl, heterocyclenyl, heterocycly, arylcycloalkyl, arylheteroalkyl, arylalkenyl and arylalkynyl, said groups may in turn be substituted by ring substituents, as well as halo, haloalkyl, hydroxyl, alkoxy, halolkoxy, amino, alklamino, dialkylamino, NO₂, mercapto, alkylthio, —N₃, —COOH, and —C(O)O-alkyl.

Substitution on a cycloalkylalkyl, heterocycloalkylalkyl, arylalkyl, or heteroarylalkyl moiety includes substitution on the ring portion and/or on the alkyl portion of the group.

When a variable appears more than once in a group, or a variable appears more than once in the structure of a formula, the variables can be the same or different.

With reference to the number of moieties (e.g., substituents, groups or rings) in a compound, unless otherwise defined, the phrases “one or more” and “at least one” mean that there can be as many moieties as chemically permitted, and the determination of the maximum number of such moieties is well within the knowledge of those skilled in the art. With respect to the compositions and methods comprising the use of the phrase “at least one” in a phrase such as “at least one cholesterol lowering agent” or “at least one H₃ antagonist/inverse agonist” means one to three cholesterol lowering agents and independently one to three H₃ receptor antagonists/inverse agonists can be administered at the same time, with preference to one of each.

As used herein, the term “composition” is intended to encompass a product comprising the specified ingredients in the specified amounts, as well as any product which results, directly or indirectly, from combination of the specified ingredients in the specified amounts.

The wavy line [symbol]
as a bond generally indicates a mixture of, or either of, the possible isomers, e.g., containing (R)- and (S)-stereochemistry. For example, 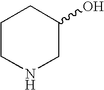
means containing both

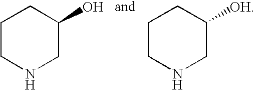

Lines drawn into the ring systems, such as, for example: 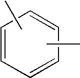
indicate that the indicated line (bond) may be attached to any of the substitutable ring carbon atoms.

It is noted that the carbon atoms for formula I may be replaced with 1 to 3 silicon atoms so long as all valency requirements are satisfied.

\textcircled{N}, for example in the structure 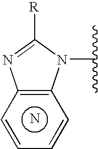
represents a nitrogen atom that is located at one of the 4 non-fused positions of the ring, i.e., positions 4, 5, 6 or 7 indicated below:

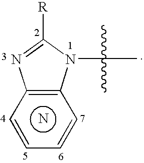

Similarly, \textcircled{2N} means that two nitrogens are located at any two of the 4 non-fused positions of the ring, e.g., the 4 and 6 positions, the 4 and 7 positions, or the 5 and 6 positions.

As well known in the art, a bond drawn from a particular atom wherein no moiety is depicted at the terminal end of the bond indicates a methyl group bound through that bond to the atom, unless stated otherwise. For example:

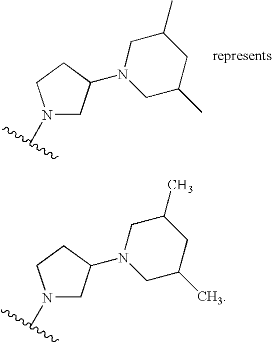

It should also be noted that any heteroatom with unsatisfied valences in the text or structural formulae herein is assumed to have the hydrogen atom or atoms to satisfy the valences.

Those skilled in the art will recognize that certain compounds in the structural formulae disclosed herein are tautomeric and all such tautomeric forms are contemplated herein as part of the present invention.

As used herein, the term “cholesterol lowering agent” means any agent capable of lowering the cholesterol level in a mammal, such as a human.

Non-limiting examples of compounds that act as lipid lowering agents include, for example, sterol absorption inhibitors, 5-α-stanol absorption inhibitors, HMG-CoA reductase inhibitors, bile acid sequestrants, nicotinic acid and/or nicotinic acid receptor agonists, agonists or activators of peroxisome proliferators-activated receptors (PPAR) etc. The term “H₃ receptor antagonist inverse agonist” is any compound that acts as an antagonist or an inverse agonist to the H₃ receptor.

The terms “combination therapy” or “therapeutic combination” means the administration of two or more therapeutic agents, such as a sterol absorption inhibitor and an H₃ receptor antagonists/inverse agonist to prevent, treat or ameloriate NAFLD or NASH. The combinations and treatments of the present invention can be administered by any suitable means which produce contact of these compounds with the site of action in the body, for example in the plasma, liver or small intestine of a subject (mammal or human or other animal). Such administration includes coadministration of these therapeutic agents in a substantially simultaneous manner, such as in a single tablet or capsule having a fixed ratio of active ingredients or in multiple, separate capsules for each therapeutic agent. Also, such administration includes use of each type of therapeutic agent in a sequential manner. In either case, the treatment using the combination therapy will provide beneficial effects in treating the condition. A potential advantage of the combination therapy disclosed herein may be a reduction in the required amount of an individual therapeutic compound or the overall total amount of therapeutic compounds that are effective in treating the condition. By using a combination of therapeutic agents, the side effects of the individual compounds can be reduced as compared to a monotherapy, which can improve patient compliance. Also, therapeutic agents can be selected to provide a broader range of complimentary effects or complimentary modes of action.

As discussed above, the therapeutic combinations and methods of the present invention may comprise one or more substituted azetidinone or substituted β-lactam sterol absorption inhibitors discussed in detail below. As used herein, “sterol absorption inhibitor” means a compound capable of inhibiting the absorption of one or more sterols, including but not limited to cholesterol, phytosterols (such as sitosterol, campesterol, stigmasterol and avenosterol), 5α-stanols (such as cholestanol, 5α-campestanol, 5α-sitostanol), and/or mixtures thereof, when administered in a therapeutically effective (sterol and/or 5α-stanol absorption inhibiting) amount to a mammal or human.

Non-limiting examples of suitable substituted azetidinones and methods of making the same include those disclosed in U.S. Pat. No. Re. 37,721, U.S. Pat. Nos. 5,306,817, 5,561,227, 5,618,707, 5,624,920, 5,631,365, 5,656,624, 5,627,176, 5,633,246, 5,661,145, 5,688,785, 5,688,787, 5,688,990, 5,698,548, 5,728,827, 5,739,321, 5,744,467, 5,756,470, 5,767,115, 5,846,966, 5,856,473, 5,886,171, 5,919,672, 6,093,812, 6,096,883, 6,133,001, 6,207,822, 6,627,757, 6,632,933, U.S. Patent Publication Nos. 2003/0105028, 2004/0180860, 2004/0180861, and 2004/0198700, N-sulfonyl-2-azetidinones such as are disclosed in U.S. Pat. No. 4,983,597, ethyl 4-(2-oxoazetidin-4-yl)phenoxy-alkanoates such as are disclosed in Ram et al., Indian J. Chem. Sect. B. 29B, 12 (1990), p. 1134-7, and diphenyl azetidinones and derivatives disclosed in U.S. Patent Publication Nos. 2002/0039774, 2002/0128252, 2002/0128253, 2002/0137689, 2004/0082561, and PCT Published Application Nos. WO 2002/066464, WO 04/000805, WO 04/005247, WO 04/000804, WO 04/000803, WO 04/014947, WO 04/087655, WO 05/009955, WO 05/023305, WO 05/021495, WO 05/021497, WO 05/044256, WO 05/042692, WO 05/033100, WO 05/030225, WO 05/047248, WO 05/046662, WO 05/061451, WO 05/061452, WO 05/062824, WO 05/02897, WO 05/000353, as well as the acetidiones disclosed in U.S. Patent Publication Nos. 2004/0077623, 2002/0137689, 2004/0067913, each of which is incorporated by reference herein.

In one embodiment, substituted azetidinones useful in the compositions, therapeutic combinations and methods of the present invention are represented by formula (I) below: 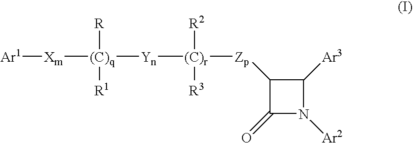
or pharmaceutically acceptable salts or solvates of the compounds of formula (I),
wherein, in formula (I) above:

Ar¹ and Ar² are independently selected from the group consisting of aryl and R⁴-substituted aryl;

Ar³ is aryl or R⁵-substituted aryl;

X, Y and Z are independently selected from the group consisting of —CH₂—, —CH(lower alkyl)- and —C(dilower alkyl)-;

R and R² are independently selected from the group consisting of —OR⁶, —O(CO)R⁶, —O(CO)OR⁹ and —O(CO)NR⁶R⁷;

R¹ and R³ are independently selected from the group consisting of hydrogen, lower alkyl and aryl;

q is 0 or 1; r is 0 or 1; m, n and p are independently selected from 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5;

R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶, —CH═CH—COOR⁶, —CF₃, —CN, —NO₂ and halogen;

R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;

R⁶, R⁷ and R⁸ are independently selected from the group consisting of hydrogen, lower alkyl, aryl and aryl-substituted lower alkyl; and

R⁹ is lower alkyl, aryl or aryl-substituted lower alkyl.

Preferably, R⁴ is 1-3 independently selected substituents, and R⁵ is preferably 1-3 independently selected substituents.

Preferred compounds of formula (I) are those in which Ar¹ is phenyl or R⁴-substituted phenyl, more preferably (4-R⁴)-substituted phenyl. Ar² is preferably phenyl or R⁴-substituted phenyl, more preferably (4-R⁴)-substituted phenyl. Ar³ is preferably R⁵-substituted phenyl, more preferably (4-R⁵)-substituted phenyl. When Ar¹ is (4-R⁴)-substituted phenyl, R⁴ is preferably a halogen. When Ar² and Ar³ are R⁴- and R⁵-substituted phenyl, respectively, R⁴ is preferably halogen or —OR⁶and R⁵ is preferably —OR⁶, wherein R⁶ is lower alkyl or hydrogen. Especially preferred are compounds wherein each of Ar¹ and Ar² is 4-fluorophenyl and Ar³ is 4-hydroxyphenyl or 4-methoxyphenyl.

X, Y and Z are each preferably —CH₂—. R¹ and R³ are each preferably hydrogen. R and R² are preferably —OR⁶ wherein R⁶ is hydrogen, or a group readily metabolizable to a hydroxyl (such as —O(CO)R⁶, —O(CO)OR⁹ and —O(CO)NR⁶R⁷, defined above).

The sum of m, n, p, q and r is preferably 2, 3 or 4, more preferably 3. Preferred are compounds wherein m, n and r are each zero, q is 1 and p is 2.

Also preferred are compounds of formula (I) in which p, q and n are each zero, r is 1 and m is 2 or 3. More preferred are compounds wherein m, n and r are each zero, q is 1, p is 2, Z is —CH₂— and R is —OR⁶, especially when R⁶ is hydrogen.

Also more preferred are compounds of formula (I) wherein p, q and n are each zero, r is 1, m is 2, X is —CH₂— and R² is —OR⁶, especially when R⁶ is hydrogen.

Another group of preferred compounds of formula (I) is that in which Ar¹ is phenyl or R⁴-substituted phenyl, Ar² is phenyl or R⁴-substituted phenyl and Ar³ is R⁵-substituted phenyl. Also preferred are compounds in which Ar¹ is phenyl or R⁴-substituted phenyl, Ar² is phenyl or R⁴-substituted phenyl, Ar³ is R⁵-substituted phenyl, and the sum of m, n, p, q and r is 2, 3 or 4, more preferably 3. More preferred are compounds wherein Ar¹ is phenyl or R⁴-substituted phenyl, Ar² is phenyl or R⁴-substituted phenyl, Ar³ is R⁵-substituted phenyl, and wherein m, n and r are each zero, q is 1 and p is 2, or wherein p, q and n are each zero, r is 1 and m is 2 or 3.

In a preferred embodiment, a substituted azetidinone of formula (I) useful in the compositions, therapeutic combinations and methods of the present invention is represented by formula (II) (ezetimibe) below: 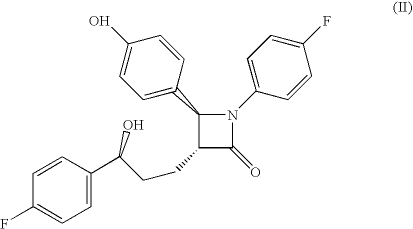
or pharmaceutically acceptable salts or solvates of the compound of formula (II). The compound of formula (II) can be in anhydrous or hydrated form. A product containing ezetimibe compound is commercially available as ZETIA® ezetimibe formulation from MSP Pharmaceuticals.

Compounds of formula I can be prepared by a variety of methods well know to those skilled in the art, for example such as are disclosed in U.S. Pat. No. Re. 37,721, U.S. Pat. Nos. 5,631,365, 5,767,115, 5,846,966, 6,207,822, PCT Patent Application No. 02/079174, and PCT Patent Application WO 93/02048, each of which is incorporated herein by reference.

Alternative substituted azetidinones useful in the compositions, therapeutic combinations and methods of the present invention are represented by formula (III) below: 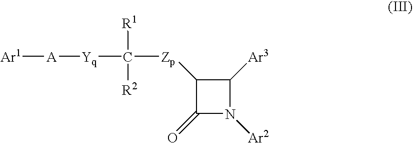
or a pharmaceutically acceptable salt thereof or a solvate thereof, wherein, in formula (III) above:

Ar¹ is R³-substituted aryl;

Ar² is R⁴-substituted aryl;

Ar³ is R⁵-substituted aryl;

Y and Z are independently selected from the group consisting of —CH₂—, —CH(lower alkyl)- and —C(dilower alkyl)-;

A is selected from —O—, —S—, —S(O)— or —S(O)₂—;

R¹ is selected from the group consisting of —OR⁶, —O(CO)R⁶, —O(CO)OR⁹ and —O(CO)NR⁶R⁷; R² is selected from the group consisting of hydrogen, lower alkyl and aryl; or R¹ and R² together are ═O;

q is 1, 2 or 3;

p is 0, 1, 2, 3 or 4;

R⁵ is 1-3 substituents independently selected from the group consisting of —OR⁶, —O(CO)R⁶, —O(CO)OR⁹, —O(CH₂)₁₋₅OR⁹, —O(CO)NR⁶R⁷, —NR⁶R⁷, —NR⁶(CO)R⁷, —NR⁶(CO)OR⁹, —NR⁶(CO)NR⁷R⁸, —NR⁶SO₂-lower alkyl, —NR⁶SO₂-aryl, —CONR⁶R⁷, —COR⁶, —SO₂NR⁶R⁷, S(O)₀₋₂-alkyl, S(O)₀₋₂-aryl, —O(CH₂)₁₋₁₀—COOR⁶, —O(CH₂)₁₋₁₀CONR⁶R⁷, o-halogeno, m-halogeno, o-lower alkyl, m-lower alkyl, -(lower alkylene)-COOR⁶, and —CH═CH—COOR⁶;

R³ and R⁴ are independently 1-3 substituents independently selected from the group consisting of R⁵, hydrogen, p-lower alkyl, aryl, —NO₂, —CF₃ and p-halogeno;

R⁶, R⁷ and R⁸ are independently selected from the group consisting of hydrogen, lower alkyl, aryl and aryl-substituted lower alkyl; and R⁹ is lower alkyl, aryl or aryl-substituted lower alkyl.

Preferred compounds of formula III include those in which Ar¹ is R³-substituted phenyl, especially (4-R³)-substituted phenyl. Ar² is preferably R⁴-substituted phenyl, especially (4-R⁴)-substituted phenyl. Ar³ is preferably R⁵-substituted phenyl, especially (4-R⁵)-substituted phenyl. Mono-substitution of each of Ar¹, Ar² and Ar³ is preferred.

Y and Z are each preferably —CH₂—. R² is preferably hydrogen. R¹ is preferably —OR⁶ wherein R⁶ is hydrogen, or a group readily metabolizable to a hydroxyl (such as —O(CO)R⁶, —O(CO)OR⁹ and —O(CO)NR⁶R⁷, defined above). Also preferred are compounds wherein R¹ and R² together are ═O.

The sum of q and p is preferably 1 or 2, more preferably 1. Preferred are compounds wherein p is zero and q is 1. More preferred are compounds wherein p is zero, q is 1, Y is —CH₂— and R¹ is —OR⁶, especially when R⁶ is hydrogen.

Another group of preferred compounds is that in which Ar¹ is R³-substituted phenyl, Ar² is R⁴-substituted phenyl and Ar³ is R⁵-substituted phenyl.

Also preferred are compounds wherein Ar¹ is R³-substituted phenyl, Ar² is R⁴-substituted phenyl, Ar³ is R⁵-substituted phenyl, and the sum of p and q is 1 or 2, especially 1. More preferred are compounds wherein Ar¹ is R³-substituted phenyl, Ar² is R⁴-substituted phenyl, Ar³ is R⁵-substituted phenyl, p is zero and q is 1.

A is preferably —O—.

R³ is preferably —COOR⁶, —CONR⁶R⁷, —COR⁶, —SO₂NR⁶R⁷, S(O)₀₋₂-alkyl, S(O)₀₋₂-aryl, NO₂ or halogeno. A more preferred definition for R³ is halogeno, especially fluoro or chloro.

R⁴ is preferably hydrogen, lower alkyl, —OR⁶, —O(CO)R⁶, —O(CO)OR⁹, —O(CO)NR⁶R⁷, —NR⁶R⁷, COR⁶ or halogeno, wherein R⁶ and R⁷ are preferably independently hydrogen or lower alkyl, and R⁹ is preferably lower alkyl. A more preferred definition for R⁴ is hydrogen or halogeno, especially fluoro or chloro.

R⁵ is preferably —OR⁶, —O(CO)R⁶, —O(CO)OR⁹, —O(CO)NR⁶R⁷, —NR⁶R⁷, -(lower alkylene)-COOR⁶ or —CH═CH—COOR⁶, wherein R⁶ and R⁷ are preferably independently hydrogen or lower alkyl, and R⁹ is preferably lower alkyl. A more preferred definition for R⁵ is —OR⁶, -(lower alkylene)-COOR⁶ or —CH═CH—COOR⁶, wherein R⁶ is preferably hydrogen or lower alkyl.

Methods for making compounds of Formula III are well known to those skilled in the art. Non-limiting examples of suitable methods are disclosed in U.S. Pat. No. 5,688,990, which is incorporated herein by reference.

In another embodiment, substituted azetidinones useful in the compositions, therapeutic combinations and methods of the present invention are represented by formula (IV): 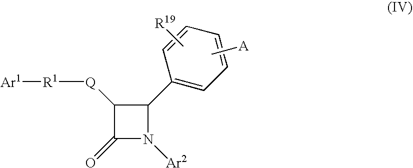
or a pharmaceutically acceptable salt thereof or a solvate thereof, wherein, in formula (IV) above:

A is selected from the group consisting of R²-substituted heterocycloalkyl, R²-substituted heteroaryl, R²-substituted benzofused heterocycloalkyl, and R²-substituted benzofused heteroaryl;

Ar¹ is aryl or R³-substituted aryl;

Ar² is aryl or R⁴-substituted aryl;

Q is a bond or, with the 3-position ring carbon of the azetidinone, forms the spiro group 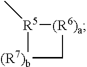
and

R¹ is selected from the group consisting of:
—(CH₂)_{q}—, wherein q is 2-6, provided that when Q forms a spiro ring, q can also be zero or 1;
—(CH₂)ₑ-G-(CH₂)ᵣ—, wherein G is —O—, —C(O)—, phenylene, —NR⁸— or —S(O)₀₋₂—, e is 0-5 and r is 0-5, provided that the sum of e and r is 1-6;
—(C₂-C₆ alkenylene)-; and
—(CH₂)_{f}—V—(CH₂)_{g}—, wherein V is C₃-C₆ cycloalkylene, f is 1-5 and g is 0-5, provided that the sum of f and g is 1-6;

R⁵ is selected from:

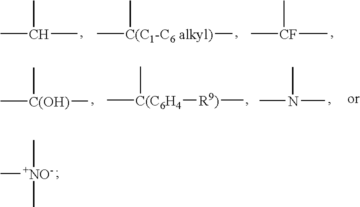

R⁶ and R⁷ are independently selected from the group consisting of —CH₂—, —CH(C₁-C₆ alkyl)-, —C(di-(C₁-C₆)alkyl), —CH═CH— and —C(C₁-C₆ alkyl)═CH—; or R⁵ together with an adjacent R⁶, or R⁵ together with an adjacent R⁷, form a —CH═CH— or a —CH═C(C₁-C₆ alkyl)- group;

a and b are independently 0, 1, 2 or 3, provided both are not zero; provided that when R⁶ is —CH═CH— or —C(C₁-C₆ alkyl)═CH—, a is 1; provided that when R⁷ is —CH═CH— or —C(C₁-C₆ alkyl)═CH—, b is 1; provided that when a is 2 or 3, the R⁶'s can be the same or different; and provided that when b is 2 or 3, the R⁷'s can be the same or different;

and when Q is a bond, R¹ also can be selected from:

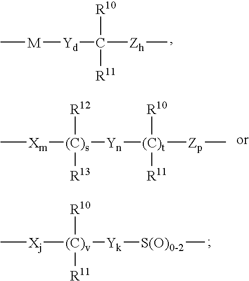

where M is —O—, —S—, —S(O)— or —S(O)₂—;

X, Y and Z are independently selected from the group consisting of —CH₂—, —CH(C₁-C₆ alkyl)- and —C(di-(C₁-C₆)alkyl);

R¹⁰ and R¹² are independently selected from the group consisting of —OR¹⁴, —O(CO)R¹⁴, —O(CO)OR¹⁶ and —O(CO)NR¹⁴R¹⁵;

R¹¹ and R¹³ are independently selected from the group consisting of hydrogen, (C₁-C₆)alkyl and aryl; or R¹⁰ and R¹¹ together are ═O, or R¹² and R¹³ together are ═O;

d is 1, 2 or 3;

h is 0, 1, 2, 3 or 4;

s is 0 or 1; t is 0 or 1; m, n and p are independently 0-4; provided that at least one of s and t is 1, and the sum of m, n, p, s and t is 1-6; provided that when p is 0 and t is 1, the sum of m, s and n is 1-5; and provided that when p is 0 and s is 1, the sum of m, t and n is 1-5;

v is 0 or 1;

j and k are independently 1-5, provided that the sum of j, k and v is 1-5;

R² is 1-3 substituents on the ring carbon atoms selected from the group consisting of hydrogen, (C₁-C₁₀)alkyl, (C₂-C₁₀)alkenyl, (C₂-C₁₀)alkynyl, (C₃-C₆)cycloalkyl, (C₃-C₆)cycloalkenyl, R¹⁷-substituted aryl, R¹⁷-substituted benzyl, R¹⁷-substituted benzyloxy, R¹⁷-substituted aryloxy, halogeno, —NR¹⁴R¹⁵, NR¹⁴R¹⁵(C₁-C₆ alkylene)-, NR¹⁴R¹⁵C(O)(C₁-C₆ alkylene)-, —NHC(O)R¹⁶, OH, C₁-C₆ alkoxy, —OC(O)R¹⁶, —COR¹⁴, hydroxy(C₁-C₆)alkyl, (C₁-C₆)alkoxy(C₁-C₆)alkyl, NO₂, —S(O)₀₋₂R¹⁶, —SO₂NR¹⁴R¹⁵ and —(C₁-C₆ alkylene)COOR¹⁴; when R² is a substituent on a heterocycloalkyl ring, R² is as defined, or is ═O or 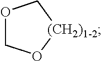
and, where R² is a substituent on a substitutable ring nitrogen, it is hydrogen, (C₁-C₆)alkyl, aryl, (C₁-C₆)alkoxy, aryloxy, (C₁-C₆)alkylcarbonyl, arylcarbonyl, hydroxy, —(CH₂)₁₋₆CONR¹⁸R¹⁸,

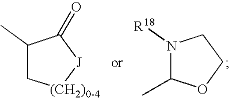

wherein J is —O—, —NH—, —NR¹⁸— or —CH₂—;

R³ and R⁴ are independently selected from the group consisting of 1-3 substituents independently selected from the group consisting of (C₁-C₆)alkyl, —OR¹⁴, —O(CO)R¹⁴, —O(CO)OR¹⁶, —O(CH₂)₁₋₅OR¹⁴, —O(CO)NR¹⁴R¹⁵, —NR¹⁴R¹⁵, —NR¹⁴(CO)R¹⁵, —NR¹⁴(CO)OR¹⁶, —NR¹⁴(CO)NR¹⁵R¹⁹, —NR¹⁴SO₂R¹⁶, —COOR¹⁴, —CONR¹⁴R¹⁵, —COR¹⁴, —SO₂NR¹⁴R¹⁵, S(O)₀₋₂R¹⁶, —O(CH₂)₁₋₁₀—COOR¹⁴, —O(CH₂)₁₋₁₀CONR¹⁴R¹⁵, —(C₁-C₆ alkylene)-COOR¹⁴, —CH═CH—COOR¹⁴, —CF₃, —CN, —NO₂ and halogen;

R⁸ is hydrogen, (C₁-C₆)alkyl, aryl(C₁-C₆)alkyl, —C(O)R¹⁴ or —COOR¹⁴;

R⁹ and R¹⁷ are independently 1-3 groups independently selected from the group consisting of hydrogen, (C₁-C₆)alkyl, (C₁-C₆)alkoxy, —COOH, NO₂, —NR¹⁴R¹⁵, OH and halogeno;

R¹⁴ and R¹⁵ are independently selected from the group consisting of hydrogen, (C₁-C₆)alkyl, aryl and aryl-substituted (C₁-C₆)alkyl;

R¹⁶ is (C₁-C₆)alkyl, aryl or R¹⁷-substituted aryl;

R¹⁸ is hydrogen or (C₁-C₆)alkyl; and

R¹⁹ is hydrogen, hydroxy or (C₁-C₆)alkoxy.

Methods for making compounds of formula IV are well known to those skilled in the art. Non-limiting examples of suitable methods are disclosed in U.S. Pat. No. 5,656,624, which is incorporated herein by reference.

As used in formula (IV) above, “A” is preferably an R²-substituted, 6-membered heterocycloalkyl ring containing 1 or 2 nitrogen atoms. Preferred heterocycloalkyl rings are piperidinyl, piperazinyl and morpholinyl groups. The ring “A” is preferably joined to the phenyl ring through a ring nitrogen. Preferred R² substituents are hydrogen and lower alkyl. R¹⁹ is preferably hydrogen.

Ar² is preferably phenyl or R⁴-phenyl, especially (4-R⁴)-substituted phenyl. Preferred definitions of R⁴ are lower alkoxy, especially methoxy, and halogeno, especially fluoro.

Ar¹ is preferably phenyl or R³-substituted phenyl, especially (4-R³)-substituted phenyl.

There are several preferred definitions for the —R¹-Q- combination of variables:

Q is a bond and R¹ is lower alkylene, preferably propylene;

Q is a spiro group as defined above, wherein preferably R⁶ and R⁷ are each ethylene and R⁵ is

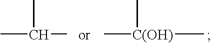

Q is a bond and R¹ is 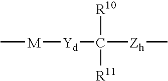
wherein the variables are chosen such that R¹ is —O—CH₂—CH(OH)—;

Q is a bond and R¹ is 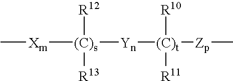
wherein the variables are chosen such that R¹ is —CH(OH)—(CH₂)₂—; and

Q is a bond and R¹ is 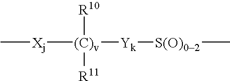
wherein the variables are chosen such that R¹ is —CH(OH)—CH₂—S(O)₀₋₂—.

In another embodiment, substituted azetidinones useful in the compositions, therapeutic combinations and methods of the present invention are represented by formula (V): 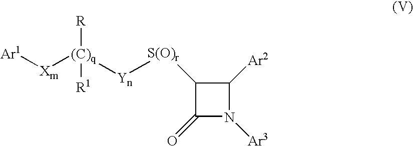
or a pharmaceutically acceptable salt thereof or a solvate thereof, wherein, in formula (V) above:

Ar¹ is aryl, R¹⁰-substituted aryl, heteroaryl or R¹⁰-substituted heteroaryl;

Ar² is aryl or R⁴-substituted aryl;

Ar³ is aryl or R⁵-Substituted aryl;

X and Y are independently selected from the group consisting of —CH₂—, —CH(lower alkyl)- and —C(dilower alkyl)-;

R is —OR⁶, —O(CO)R⁶, —O(CO)OR⁹ or —O(CO)NR⁶R⁷; R¹ is hydrogen, lower alkyl or aryl; or R and R¹ together are ═O;

q is 0 or 1;

r is 0, 1 or 2;

m and n are independently 0, 1, 2, 3, 4 or 5; provided that the sum of m, n and q is 1, 2, 3, 4 or 5;

R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;

R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, —CF₃, —CN, —NO₂, halogen, -(lower alkylene)COOR⁶ and —CH═CH—COOR⁶;

R⁶, R⁷ and R⁸ are independently selected from the group consisting of hydrogen, lower alkyl, aryl and aryl-substituted lower alkyl;

R⁹ is lower alkyl, aryl or aryl-substituted lower alkyl; and

R¹⁰ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, —S(O)₀₋₂R⁹, —O(CH₂)₁₋₁₀—COOR⁶, —O(CH₂)₁₋₁₀CONR⁶R⁷, —CF₃, —CN, —NO₂ and halogen.

Within the scope of Formula V, there are included two preferred structures. In formula VA, q is zero and the remaining variables are as defined above, and in formula VB, q is 1 and the remaining variables are as defined above:

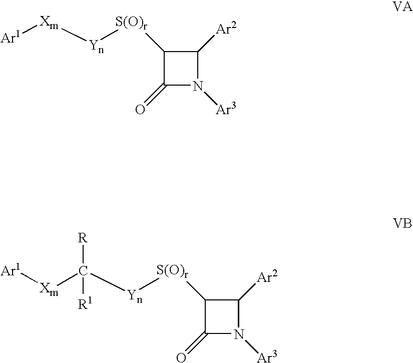

R⁴, R⁵ and R¹⁰ are each preferably 1-3 independently selected substituents as set forth above. Preferred are compounds of Formula (V) wherein Ar¹ is phenyl, R¹⁰-substituted phenyl or thienyl, especially (4-R¹⁰)-substituted phenyl or thienyl. Ar² is preferably R⁴-substituted phenyl, especially (4-R⁴)-substituted phenyl. Ar³ is preferably phenyl or R⁵-substituted phenyl, especially (4-R⁵)-substituted phenyl. When Ar¹ is R¹⁰-substituted phenyl, R¹⁰ is preferably halogeno, especially fluoro. When Ar² is R⁴-substituted phenyl, R⁴ is preferably —OR⁶, especially wherein R⁶ is hydrogen or lower alkyl. When Ar³ is R⁵-substituted phenyl, R⁵ is preferably halogeno, especially fluoro. Especially preferred are compounds of formula (V) wherein Ar¹ is phenyl, 4-fluorophenyl or thienyl, Ar² is 4-(alkoxy or hydroxy)phenyl, and Ar³ is phenyl or 4-fluorophenyl.

X and Y are each preferably —CH₂—. The sum of m, n and q is preferably 2, 3 or 4, more preferably 2. When q is 1, n is preferably 1 to 5.

Preferences for X, Y, Ar¹, Ar² and Ar³ are the same in each of formulae (VA) and (VB).

In compounds of formula (VA), the sum of m and n is preferably 2, 3 or 4, more preferably 2. Also preferred are compounds wherein the sum of m and n is 2, and r is 0 or 1.

In compounds of formula (VB), the sum of m and n is preferably 1, 2 or 3, more preferably 1. Especially preferred are compounds wherein m is zero and n is 1. R¹ is preferably hydrogen and R is preferably —OR⁶ wherein R⁶ is hydrogen, or a group readily metabolizable to a hydroxyl (such as —O(CO)R⁶, —O(CO)OR⁹ and —O(CO)NR⁶R⁷, defined above), or R and R¹ together form a ═O group.

Methods for making compounds of formula V are well known to those skilled in the art. Non-limiting examples of suitable methods are disclosed in U.S. Pat. No. 5,624,920, which is incorporated herein by reference.

In another embodiment, substituted azetidinones useful in the compositions, therapeutic combinations and methods of the present invention are represented by formula (VI): 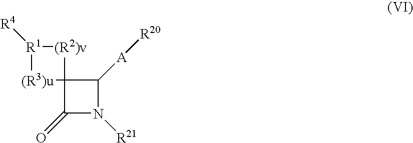
or a pharmaceutically acceptable salt thereof or a solvate thereof, wherein:

R¹ is

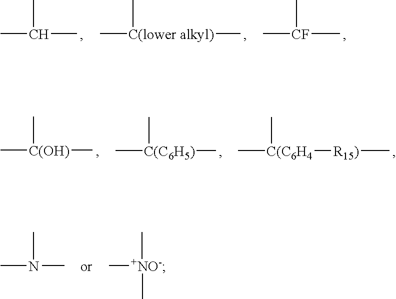

R² and R³ are independently selected from the group consisting of:

—CH₂—, —CH(lower alkyl)-, —C(di-lower alkyl)-, —CH═CH— and —C(lower alkyl)═CH—; or R¹ together with an adjacent R², or R¹ together with an adjacent R³, form a —CH═CH— or a —CH═C(lower alkyl)- group;

u and v are independently 0, 1, 2 or 3, provided both are not zero; provided that when R² is —CH═CH— or —C(lower alkyl)═CH—, v is 1; provided that when R³ is —CH═CH— or —C(lower alkyl)═CH—, u is 1; provided that when v is 2 or 3, the R²'s can be the same or different; and provided that when u is 2 or 3, the R³'s can be the same or different;

R⁴ is selected from B—(CH₂)ₘC(O)—, wherein m is 0, 1, 2, 3, 4 or 5;

B—(CH₂)_{q}—, wherein q is 0, 1, 2, 3, 4, 5 or 6; B—(CH₂)ₑ-Z-(CH₂)ᵣ—, wherein Z is —O—, —C(O)—, phenylene, —N(R⁸)— or —S(O)₀₋₂—, e is 0, 1, 2, 3, 4 or 5 and r is 0, 1, 2, 3, 4 or 5, provided that the sum of e and r is 0, 1, 2, 3, 4, 5 or 6; B—(C₂-C₆ alkenylene)-; B—(C₄-C₆ alkadienylene)-; B—(CH₂)ₜ-Z-(C₂-C₆ alkenylene)-, wherein Z is as defined above, and wherein t is 0, 1, 2 or 3, provided that the sum of t and the number of carbon atoms in the alkenylene chain is 2, 3, 4, 5 or 6; B—(CH₂)_{f}—V—(CH₂)_{g}—, wherein V is C₃-C₆ cycloalkylene, f is 1, 2, 3, 4 or 5 and g is 0, 1, 2, 3, 4 or 5, provided that the sum of f and g is 1, 2, 3, 4, 5 or 6; B—(CH₂)ₜ—V—(C₂-C₆ alkenylene)- or B—(C₂-C₆ alkenylene)-V—(CH₂)ₜ—, wherein V and t are as defined above, provided that the sum of t and the number of carbon atoms in the alkenylene chain is 2, 3, 4, 5 or 6;

B—(CH₂)ₐ-Z-(CH₂)_{b}—V—(CH₂)_{d}—, wherein Z and V are as defined above and a, b and d are independently 0, 1, 2, 3, 4, 5 or 6, provided that the sum of a, b and d is 0, 1, 2, 3, 4, 5 or 6; or T-(CH₂)ₛ—, wherein T is cycloalkyl of 3-6 carbon atoms and s is 0, 1, 2, 3, 4, 5 or 6; or

R¹ and R⁴ together form the group

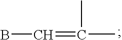

B is selected from indanyl, indenyl, naphthyl, tetrahydronaphthyl, heteroaryl or W-substituted heteroaryl, wherein heteroaryl is selected from the group consisting of pyrrolyl, pyridinyl, pyrimidinyl, pyrazinyl, triazinyl, imidazolyl, thiazolyl, pyrazolyl, thienyl, oxazolyl and furanyl, and for nitrogen-containing heteroaryls, the N-oxides thereof, or

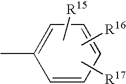

W is 1 to 3 substituents independently selected from the group consisting of lower alkyl, hydroxy lower alkyl, lower alkoxy, alkoxyalkyl, alkoxyalkoxy, alkoxycarbonylalkoxy, (lower alkoxyimino)-lower alkyl, lower alkanedioyl, lower alkyl lower alkanedioyl, allyloxy, —CF₃, —OCF₃, benzyl, R⁷-benzyl, benzyloxy, R⁷-benzyloxy, phenoxy, R⁷-phenoxy, dioxolanyl, NO₂, —N(R⁸)(R⁹), N(R⁸)(R⁹)-lower alkylene-, N(R⁸)(R⁹)-lower alkylenyloxy-, OH, halogeno, —CN, —N₃, —NHC(O)OR¹⁰, —NHC(O)R¹⁰, R¹¹O₂SNH—, (R¹¹O₂S)₂N—, —S(O)₂NH₂, —S(O)₀₋₂R⁸, tert-butyldimethyl-silyloxymethyl, —C(O)R¹², —COOR¹⁹, —CON(R⁸)(R⁹), —CH═CHC(O)R¹², -lower alkylene-C(O)R¹², R¹⁰C(O)(lower alkylenyloxy)-, N(R⁸)(R⁹)C(O)(lower alkylenyloxy)- and 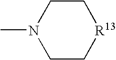
for substitution on ring carbon atoms,
and the substituents on the substituted heteroaryl ring nitrogen atoms, when present, are selected from the group consisting of lower alkyl, lower alkoxy, —C(O)OR¹⁰, —C(O)R¹⁰, OH, N(R⁸)(R⁹)-lower alkylene-, N(R⁸)(R⁹)-lower alkylenyloxy-, —S(O)₂NH₂ and 2-(trimethylsilyl)-ethoxymethyl;

R⁷ is 1-3 groups independently selected from the group consisting of lower alkyl, lower alkoxy, —COOH, NO₂, —N(R⁸)(R⁹), OH, and halogeno;

R⁸ and R⁹ are independently selected from H or lower alkyl;

R¹⁰ is selected from lower alkyl, phenyl, R⁷-phenyl, benzyl or R⁷-benzyl;

R¹¹ is selected from OH, lower alkyl, phenyl, benzyl, R⁷-phenyl or R⁷-benzyl;

R¹² is selected from H, OH, alkoxy, phenoxy, benzyloxy, 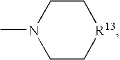
—N(R⁸)(R⁹), lower alkyl, phenyl or R⁷-phenyl;

R¹³ is selected from —O—, —CH₂—, —NH—, —N(lower alkyl)- or —NC(O)R¹⁹;

R¹⁵, R¹⁶ and R¹⁷ are independently selected from the group consisting of H and the groups defined for W; or R¹⁵ is hydrogen and R¹⁶ and R¹⁷, together with adjacent carbon atoms to which they are attached, form a dioxolanyl ring;

R¹⁹ is H, lower alkyl, phenyl or phenyl lower alkyl; and

R²⁰ and R²¹ are independently selected from the group consisting of phenyl, W-substituted phenyl, naphthyl, W-substituted naphthyl, indanyl, indenyl, tetrahydronaphthyl, benzodioxolyl, heteroaryl, W-substituted heteroaryl, benzofused heteroaryl, W-substituted benzofused heteroaryl and cyclopropyl, wherein heteroaryl is as defined above.

One group of preferred compounds of formula VI is that in which R²¹ is selected from phenyl, W-substituted phenyl, indanyl, benzofuranyl, benzodioxolyl, tetrahydronaphthyl, pyridyl, pyrazinyl, pyrimidinyl, quinolyl or cyclopropyl,

wherein W is lower alkyl, lower alkoxy, OH, halogeno, —N(R⁸)(R⁹), —NHC(O)OR¹⁰, —NHC(O)R¹⁰, NO₂, —CN, —N₃, —SH, —S(O)₀₋₂-(lower alkyl), —COOR¹⁹, —CON(R⁸)(R⁹), —COR¹², phenoxy, benzyloxy, —OCF₃, —CH═C(O)R¹² or tert-butyldimethylsilyloxy, wherein R⁸, R⁹, R¹⁰, R¹² and R¹⁹ are as defined for formula IV. When W is 2 or 3 substituents, the substituents can be the same or different.

Another group of preferred compounds of formula VI is that in which R²⁰ is phenyl or W-substituted phenyl, wherein preferred meanings of W are as defined above for preferred definitions of R²¹.

More preferred are compounds of formula VI wherein R²⁰ is phenyl or W-substituted phenyl and R²¹ is phenyl, W-substituted phenyl, indanyl, benzofuranyl, benzodioxolyl, tetrahydronaphthyl, pyridyl, pyrazinyl, pyrimidinyl, quinolyl or cyclopropyl; W is lower alkyl, lower alkoxy, OH, halogeno, —N(R⁸)(R⁹), —NHC(O)OR¹⁰, —NHC(O)R¹⁰, NO₂, —CN, —N₃, —SH, —S(O)₀₋₂-(lower alkyl), —COOR¹⁹, —CON(R⁸)(R⁹), —COR¹², phenoxy, benzyloxy, —CH═CHC(O)R₁₂, —OCF₃ or tert-butyl-dimethyl-silyloxy, wherein when W is 2 or 3 substituents, the substituents can be the same or different, and wherein R⁸, R⁹, R¹⁰, R¹² and R¹⁹ are as defined in formula VI.

Also preferred are compounds of formula VI wherein R¹ is

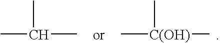

Another group of preferred compounds of formula VI is in which R² and R³ are each —CH₂— and the sum of u and v is 2, 3 or 4, with u=v=2 being more preferred.

R⁴ is preferably B—(CH₂)_{q}— or B—(CH₂)ₑ-Z-(CH₂)ᵣ—, wherein B, Z, q, e and r are as defined above. B is preferably 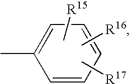
wherein R¹⁶ and R¹⁷ are each hydrogen and wherein R¹⁵ is preferably H, OH, lower alkoxy, especially methoxy, or halogeno, especially chloro.

Preferably Z is —O—, e is 0, and r is 0.

Preferably q is 0-2.

R²⁰ is preferably phenyl or W-substituted phenyl.

Preferred W substituents for R²⁰ are lower alkoxy, especially methoxy and ethoxy, OH, and —C(O)R¹², wherein R¹² is preferably lower alkoxy.

Preferably R²¹ is selected from phenyl, lower alkoxy-substituted phenyl and F-phenyl.

Especially preferred are compounds of formula VI wherein R¹ is 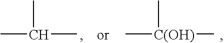
R² and R³ are each —CH₂—, u=v=2, R⁴ is B—(CH₂)_{q}—, wherein B is phenyl or phenyl substituted by lower alkoxy or chloro, q is 0-2, R²⁰ is phenyl, OH-phenyl, lower alkoxy-substituted phenyl or lower alkoxycarbonyl-substituted phenyl, and R²¹ is phenyl, lower alkoxy-substituted phenyl or F-phenyl.

An example of another useful compound of formula VI is shown below in formula VIa:

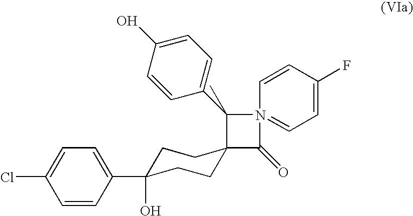

Methods for making compounds of Formula VI are well known to those skilled in the art. Non-limiting examples of suitable methods are disclosed in U.S. Pat. No. 5,698,548, which is incorporated herein by reference.

In another embodiment, substituted azetidinones useful in the compositions, therapeutic combinations and methods of the present invention are represented by Formulas (VIIA) and (VIIB): 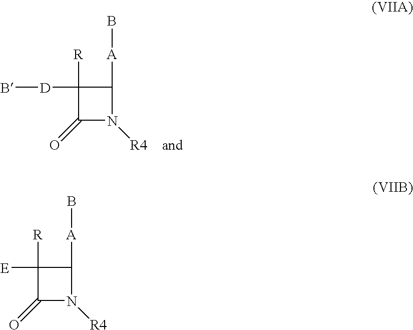
or a pharmaceutically acceptable salt or solvate thereof,
wherein:

A is —CH═CH—, —C≡C— or —(CH₂)ₚ— wherein p is 0, 1 or 2;

B is

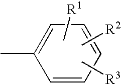

B′ is

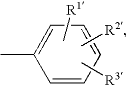

D is —(CH₂)ₘC(O)— or —(CH₂)_{q}— wherein m is 1, 2, 3 or 4 and q is 2, 3 or 4;

E is C₁₀ to C₂₀ alkyl or —C(O)—(C₉ to C₁₉)-alkyl, wherein the alkyl is straight or branched, saturated or containing one or more double bonds;

R is hydrogen, C₁-C₁₅ alkyl, straight or branched, saturated or containing one or more double bonds, or B—(CH₂)ᵣ—, wherein r is 0, 1, 2, or 3;

R¹, R², R³, R^{1′}, R^{2′}, and R^{3′} are independently selected from the group consisting of hydrogen, lower alkyl, lower alkoxy, carboxy, NO₂, NH₂, OH, halogeno, lower alkylamino, dilower alkylamino, —NHC(O)OR⁵, R⁶O₂SNH— and —S(O)₂NH₂;

R⁴ is 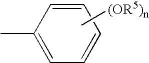
wherein n is 0, 1, 2 or 3;

R⁵ is lower alkyl; and

R⁶ is OH, lower alkyl, phenyl, benzyl or substituted phenyl wherein the substituents are 1-3 groups independently selected from the group consisting of lower alkyl, lower alkoxy, carboxy, NO₂, NH₂, OH, halogeno, lower alkylamino and dilower alkylamino; or a pharmaceutically acceptable salt thereof or a solvate thereof.

In another embodiment, sterol absorption inhibitors useful in the compositions, therapeutic combinations and methods of the present invention are represented by formula (VIII): 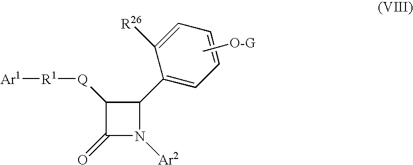
or a pharmaceutically acceptable salt thereof or a solvate thereof, wherein, in formula (VIII) above,

R²⁶ is H or OG¹;

G and G¹ are independently selected from the group consisting of H, 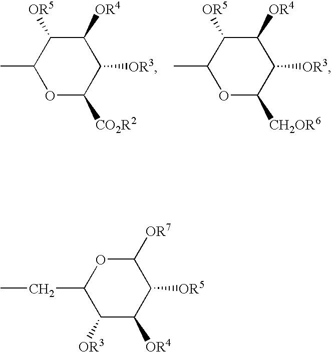
and 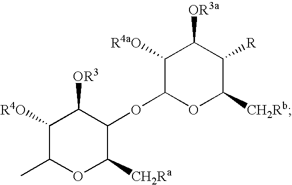
provided that when R²⁶ is H or OH, G is not H;

R⁶, R^{a} and R^{b} are independently selected from the group consisting of H, —OH, halogeno, —NH₂, azido, (C₁-C₆)alkoxy(C₁-C₆)-alkoxy or —W—R³⁰;

W is independently selected from the group consisting of —NH—C(O)—, —O—C(O)—, —O—C(O)—N(R³¹)—, —NH—C(O)—N(R³¹)— and —O—C(S)—N(R³¹)—;

R² and R⁶ are independently selected from the group consisting of H, (C₁-C₆)alkyl, aryl and aryl(C₁-C₆)alkyl;

R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;

R³⁰ is selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;

R³¹ is selected from the group consisting of H and (C₁-C₄)alkyl;

T is selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;

R³² is independently selected from 1-3 substituents independently selected from the group consisting of halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

Ar¹ is aryl or R¹⁰-substituted aryl;

Ar² is aryl or R¹¹-substituted aryl;

Q is a bond or, with the 3-position ring carbon of the azetidinone, forms the spiro group 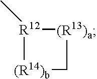
and

R¹ is selected from the group consisting of
—(CH₂)_{q}—, wherein q is 2-6, provided that when Q forms a spiro ring, q can also be zero or 1;
—(CH₂)ₑ-E-(CH₂)ᵣ—, wherein E is —O—, —C(O)—, phenylene, —NR²²— or —S(O)₀₋₂—, e is 0-5 and r is 0-5, provided that the sum of e and r is 1-6;
—(C₂-C₆)alkenylene-; and
—(CH₂)_{f}—V—(CH₂)_{g}—, wherein V is C₃-C₆ cycloalkylene, f is 1-5 and g is 0-5, provided that the sum of f and g is 1-6;

R¹² is

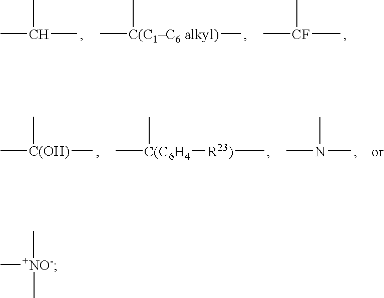

R¹³ and R¹⁴ are independently selected from the group consisting of —CH₂—, —CH(C₁-C₆ alkyl), —C(di-(C₁-C₆)alkyl), —CH═CH— and —C(C₁-C₆ alkyl)═CH—; or R¹² together with an adjacent R¹³, or R¹² together with an adjacent R¹⁴, form a —CH═CH— or a —CH═C(C₁-C₆ alkyl)- group;

a and b are independently 0, 1, 2 or 3, provided both are not zero;

provided that when R¹³ is —CH═CH— or —C(C₁-C₆ alkyl)═CH—, a is 1;

provided that when R¹⁴ is —CH═CH— or —C(C₁-C₆ alkyl)═CH—, b is 1;

provided that when a is 2 or 3, the R¹³'s can be the same or different; and

provided that when b is 2 or 3, the R¹⁴'s can be the same or different;

and when Q is a bond, R¹ also can be:

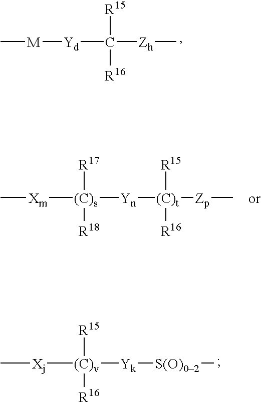

M is —O—, —S—, —S(O)— or —S(O)₂—;

X, Y and Z are independently selected from the group consisting of —CH₂—, —CH(C₁-C₆)alkyl- and —C(di-(C₁-C₆)alkyl);

R¹⁰ and R¹¹ are independently selected from the group consisting of
1-3 substituents independently selected from the group consisting of
   (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halogen;

R¹⁵ and R¹⁷ are independently selected from the group consisting of —OR¹⁹, —O(CO)R¹⁹, —O(CO)OR²¹ and —O(CO)NR¹⁹R²⁰;

R¹⁶ and R¹⁸ are independently selected from the group consisting of H, (C₁-C₆)alkyl and aryl; or R¹⁵ and R¹⁶ together are ═O, or R¹⁷ and R¹⁸ together are ═O;

d is 1, 2 or 3;

h is 0, 1, 2, 3 or 4;

s is 0 or 1; t is 0 or 1; m, n and p are independently 0-4;

provided that at least one of s and t is 1, and the sum of m, n, p, s and t is 1-6;

provided that when p is 0 and t is 1, the sum of m, s and n is 1-5; and provided that when p is 0 and s is 1, the sum of m, t and n is 1-5;

v is 0 or 1;

j and k are independently 1-5, provided that the sum of j, k and v is 1-5;

and when Q is a bond and R¹ is 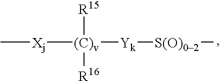
Ar¹ can also be pyridyl, isoxazolyl, furanyl, pyrrolyl, thienyl, imidazolyl, pyrazolyl, thiazolyl, pyrazinyl, pyrimidinyl or pyridazinyl;

R¹⁹ and R²⁰ are independently selected from the group consisting of H, (C₁-C₆)alkyl, aryl and aryl-substituted (C₁-C₆)alkyl;

R²¹ is (C₁-C₆)alkyl, aryl or R²⁴-substituted aryl;

R²² is H, (C₁-C₆)alkyl, aryl (C₁-C₆)alkyl, —C(O)R¹⁹ or —COOR¹⁹;

R²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and

R²⁵ is H, —OH or (C₁-C₆)alkoxy.

Methods for making compounds of formula VIII are well known to those skilled in the art. Non-limiting examples of suitable methods are disclosed in U.S. Pat. No. 5,756,470, which is incorporated herein by reference.

In another embodiment, substituted azetidinones useful in the compositions, therapeutic combinations and methods of the present invention are represented by formula (IX) below: 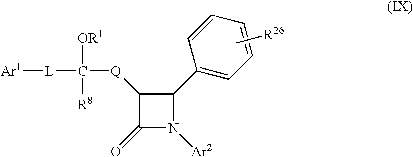
or a pharmaceutically acceptable salt or solvate thereof, wherein in Formula (IX):

R¹ is selected from the group consisting of H, G, G¹, G², —SO₃H and —PO₃H;

G is selected from the group consisting of: H, 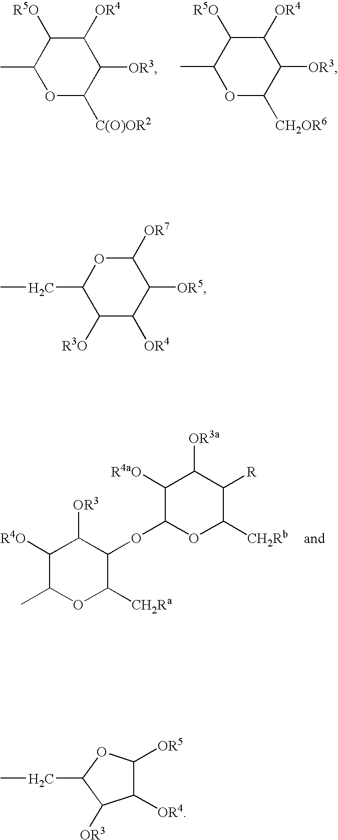
(sugar derivatives)

wherein R⁶, R^{a} and R^{b} are each independently selected from the group consisting of H, —OH, halo, —NH₂, azido, (C₁-C₆)alkoxy(C₁-C₆)alkoxy or —W—R³⁰;

W is independently selected from the group consisting of —NH—C(O)—, —O—C(O)—, —O—C(O)—N(R³¹)—, —NH—C(O)—N(R³¹)— and —O—C(S)—N(R³¹)—;

R² and R⁶ are each independently selected from the group consisting of H, (C₁-C₆)alkyl, acetyl, aryl and aryl(C₁-C₆)alkyl;

R³, R⁴, R⁵, R⁷, R^{3a} and R^{4a} are each independently selected from the group consisting of H, (C₁-C₆)alkyl, acetyl, aryl(C₁-C₆)alkyl, —C(O)(C₁-C₆)alkyl and —C(O)aryl;

R³⁰ is independently selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;

R³¹ is independently selected from the group consisting of H and (C₁-C₄)alkyl;

T is independently selected from the group consisting of phenyl, furyl, thienyl, pyrrolyl, oxazolyl, isoxazolyl, thiazolyl, isothiazolyl, benzothiazolyl, thiadiazolyl, pyrazolyl, imidazolyl and pyridyl;

R³² is independently selected from 1-3 substituents which are each independently selected from the group consisting of H, halo, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

G¹ is represented by the structure: 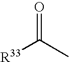
wherein R³³ is independently selected from the group consisting of unsubstituted alkyl, R³⁴-substituted alkyl, (R³⁵)(R³⁶)alkyl-,

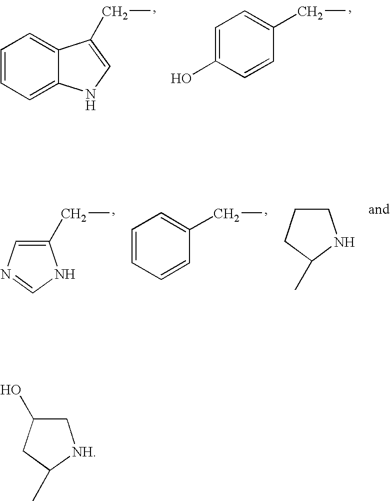

R³⁴ is one to three substituents, each R³⁴ being independently selected from the group consisting of HOOC—, HO—, HS—, (CH₃)S—, H₂N—, (NH₂)(NH)C(NH)—, (NH₂)C(O)— and HOOCCH(NH₃⁺)CH₂SS—;

R³⁵ is independently selected from the group consisting of H and NH₂—;

R³⁶ is independently selected from the group consisting of H, unsubstituted alkyl, R³⁴-substituted alkyl, unsubstituted cycloalkyl and R³⁴-substituted cycloalkyl;

G² is represented by the structure: 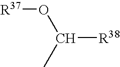
wherein R³⁷ and R³⁸ are each independently selected from the group consisting of (C₁-C₆)alkyl and aryl;

R²⁶ is one to five substituents, each R²⁶ being independently selected from the group consisting of:
a) H;
b) —OH;
c) —OCH₃;
d) fluorine;
e) chlorine;
f) —O-G;
g) —O-G¹;
h) —O-G²;
i) —SO₃H; and
j) —PO₃H;
   provided that when R¹ is H, R²⁶ is not H, —OH, —OCH₃ or —O-G;

Ar¹ is aryl, R¹⁰-substituted aryl, heteroaryl or R¹⁰-substituted heteroaryl;

Ar² is aryl, R¹¹-substituted aryl, heteroaryl or R¹¹-substituted heteroaryl;

L is selected from the group consisting of:
a) a covalent bond;
b) —(CH₂)_{q}—, wherein q is 1-6;
c) —(CH₂)ₑ-E-(CH₂)ᵣ—, wherein E is —O—, —C(O)—, phenylene, —NR²²— or —S(O)₀₋₂—, e is 0-5 and r is 0-5, provided that the sum of e and r is 1-6;
d) —C₂-C₆)alkenylene-;
e) —(CH₂)_{f}—V—(CH₂)_{g}—, wherein V is C₃-C₆cycloalkylene, f is 1-5 and g is 0-5, provided that the sum of f and g is 1-6; and
f) 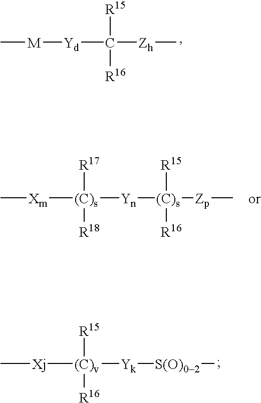
   wherein M is —O—, —S—, —S(O)— or —S(O)₂—;

X, Y and Z are each independently selected from the group consisting of —CH₂—, —CH(C₁-C₆)alkyl- and —C(di-(C₁-C₆)alkyl);

R⁸ is selected from the group consisting of H and alkyl;

R¹⁰ and R¹¹ are each independently selected from the group consisting of 1-3 substituents which are each independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halo;

R¹⁵ and R¹⁷ are each independently selected from the group consisting of —OR¹⁹, —OC(O)R¹⁹, —OC(O)OR²¹, —OC(O)NR¹⁹R²⁰;

R¹⁶ and R¹⁸ are each independently selected from the group consisting of H, (C₁-C₆)alkyl and aryl;

or R¹⁵ and R¹⁶ together are ═O, or R¹⁷ and R¹⁸ together are ═O;

d is 1, 2 or 3;

h is 0, 1, 2, 3 or 4;

s is 0 or 1;

t is 0 or 1;

m, n and p are each independently selected from 0-4;

provided that at least one of s and t is 1, and the sum of m, n, p, s and t is 1-6; provided that when p is 0 and t is 1, the sum of m, n and p is 1-5; and provided that when p is 0 and s is 1, the sum of m, t and n is 1-5;

v is 0 or 1;

j and k are each independently 1-5, provided that the sum of j, k and v is 1-5;

Q is a bond, —(CH₂)_{q}—, wherein q is 1-6, or, with the 3-position ring carbon of the azetidinone, forms the spiro group

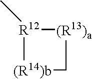

wherein R¹² is

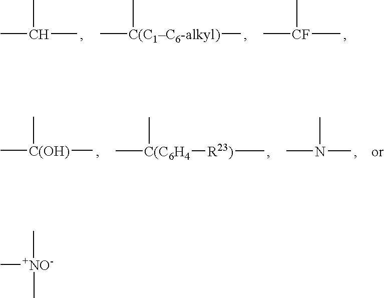

R¹³ and R¹⁴ are each independently selected from the group consisting of —CH₂—, —CH(C₁-C₆ alkyl)-, —C(di-(C₁-C₆)alkyl), —CH═CH— and —C(C₁-C₆ alkyl)═CH—; or R¹² together with an adjacent R¹³, or R¹² together with an adjacent R¹⁴, form a —CH═CH— or a —CH═C(C₁-C₆ alkyl)- group;

a and b are each independently 0, 1, 2 or 3, provided both are not zero; provided that when R¹³ is —CH═CH— or —C(C₁-C₆ alkyl)═CH—, a is 1; provided that when R¹⁴ is —CH═CH— or —C(C₁-C₆ alkyl)═CH—, b is 1; provided that when a is 2 or 3, the R¹³'s can be the same or different; and provided that when b is 2 or 3, the R¹⁴'s can be the same or different;
and when Q is a bond and L is 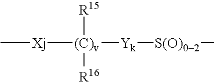
then Ar¹ can also be pyridyl, isoxazolyl, furanyl, pyrrolyl, thienyl, imidazolyl, pyrazolyl, thiazolyl, pyrazinyl, pyrimidinyl or pyridazinyl;

R¹⁹ and R²⁰ are each independently selected from the group consisting of H, (C₁-C₆)alkyl, aryl and aryl-substituted (C₁-C₆)alkyl;

R²¹ is (C₁-C₆)alkyl, aryl or R²⁴-substituted aryl;

R²² is H, (C₁-C₆)alkyl, aryl (C₁-C₆)alkyl, —C(O)R¹⁹ or —COOR¹⁹;

R²³ and R²⁴ are each independently selected from the group consisting of 1-3 substituents which are each independently selected from the group consisting of H, (C₁-C₆)alkyl, (C₁-C₆)alkoxy, —COOH, NO₂, —NR¹⁹R²⁰, —OH and halo; and

R²⁵ is H, —OH or (C₁-C₆)alkoxy.

Examples of compounds of formula (IX) which are useful in the compositions, therapeutic combinations and methods and combinations of the present invention and methods for making such compounds are disclosed in U.S. Patent Publication No. 2003/0105028 A1, filed Jun. 11, 2002, incorporated herein by reference.

An example of a useful compound of this invention is one represented by the formula X: 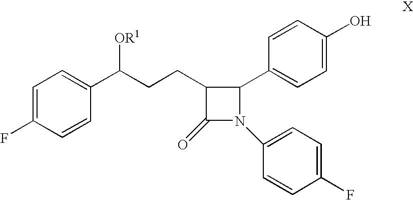
wherein R¹ is defined as above.

A more preferred compound is one represented by formula XI:

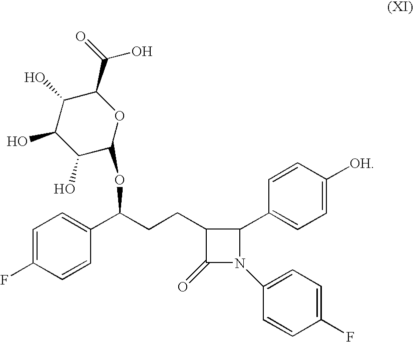

Another useful compound is represented by formula XII:

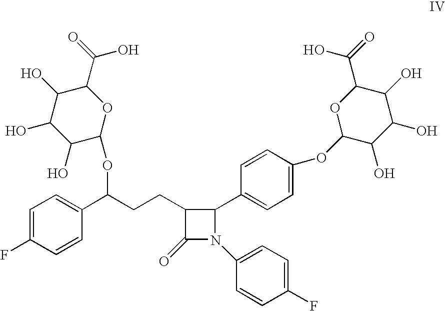

Other useful substituted azetidinone compounds include N-sulfonyl-2-azetidinones such as are disclosed in U.S. Pat. No. 4,983,597, ethyl 4-(2-oxoazetidin-4-yl)phenoxy-alkanoates such as are disclosed in Ram et al., Indian J. Chem. Sect. B. 29B, 12 (1990), p. 1134-7, and diphenyl azetidinones and derivatives disclosed in U.S. Patent Publication Nos. 2002/0039774, 2002/0128252, 2002/0128253 and 2002/0137689, and WO 2002/066464, each of which is incorporated by reference herein.

The compounds of formulae I-XII can be prepared by known methods, including the methods discussed above and, for example, WO 93/02048 describes the preparation of compounds wherein —R¹-Q- is alkylene, alkenylene or alkylene interrupted by a hetero atom, phenylene or cycloalkylene; WO 94/17038 describes the preparation of compounds wherein Q is a spirocyclic group; WO 95/08532 describes the preparation of compounds wherein —R¹-Q- is a hydroxy-substituted alkylene group; PCT/US95/03196 describes compounds wherein —R¹-Q- is a hydroxy-substituted alkylene attached to the Ar¹ moiety through an —O— or S(O)₀₋₂— group; and U.S. Pat. No. 5,633,246 describes the preparation of compounds wherein —R¹-Q- is a hydroxy-substituted alkylene group attached the azetidinone ring by a —S(O)₀₋₂— group. Each of the aforementioned documents are incorporated by reference.

Other classes of cholesterol lowering agents include the following non-limiting classes of agents: HMG-CoA reductase inhibitors; bile acid sequestrants; PPAR agonists or activators; ileal bile acid transport (“IBAT”) inhibitors (or apical sodium co-dependent bile acid transport (“ASBT”) inhibitors; nicotinic acid (niacin) and/or nicotinic acid receptor agonists; acylCoA:cholesterol O-acyltransferase (“ACAT”) inhibitors; cholesteryl ester transfer protein (“CETP”) inhibitors; probucol or derivatives thereof; low-density lipoprotein (“LDL”) receptor activators; omega 3 fatty acids (“3-PUFA”); natural water soluble fibers; plant sterols, plant stanols and/or fatty acid esters of plant stanols.

Non-limiting examples of suitable cholesterol biosynthesis inhibitors include competitive inhibitors of HMG-CoA reductase, the rate-limiting step in cholesterol biosynthesis, squalene synthase inhibitors, squalene epoxidase inhibitors and mixtures thereof. Non-limiting examples of suitable HMG-CoA reductase inhibitors include statins such as lovastatin (for example MEVACOR® which is available from Merck & Co.), pravastatin (for example PRAVACHOL® which is available from Bristol Meyers Squibb), fluvastatin, simvastatin (for example ZOCOR® which is available from Merck & Co.), atorvastatin, cerivastatin, CI-981, resuvastatin, rivastatin and pitavastatin (such as NK-104 of Negma Kowa of Japan), rosuvastatin; HMG-CoA reductase inhibitors, for example L-659,699 ((E,E)-11-[3′R-(hydroxy-methyl)-4′-oxo-2′R-oxetanyl]-3,5,7R-trimethyl-2,4-undecadienoic acid); squalene synthesis inhibitors, for example squalestatin 1; and squalene epoxidase inhibitors, for example, NB-598 ((E)-N-ethyl-N-(6,6-dimethyl-2-hepten-4-ynyl)-3-[(3,3′-bithiophen-5-yl)methoxy]benzene-methanamine hydrochloride) and other sterol biosynthesis inhibitors such as DMP-565. Preferred HMG-CoA reductase inhibitors include lovastatin, pravastatin and simvastatin. The most preferred HMG-CoA reductase inhibitor is simvastatin.

Generally, a total daily dosage of cholesterol biosynthesis inhibitor(s) can range from about 0.1 to about 160 mg per day, and preferably about 0.2 to about 80 mg/day in single or 2-3 divided doses.

Other lipid lowering agents which are contemplated by the present invention include one bile acid sequestrants. Bile acid squestrants bind bile acids in the intestine, interrupting the enterohepatic circulation of bile acids and causing an increase in the faecal excretion of steroids.

Non-limiting examples of suitable bile acid sequestrants include cholestyramine (a styrene-divinylbenzene copolymer containing quaternary ammonium cationic groups capable of binding bile acids, such as QUESTRAN® or QUESTRAN LIGHT® cholestyramine which are available from Bristol-Myers Squibb), colestipol (a copolymer of diethylenetriamine and 1-chloro-2,3-epoxypropane, such as COLESTID® tablets which are available from Pharmacia), colesevelam hydrochloride (such as WelChol® Tablets (poly(allylamine hydrochloride) cross-linked with epichlorohydrin and alkylated with 1-bromodecane and (6-bromohexyl)-trimethylammonium bromide) which are available from Sankyo), water soluble derivatives such as 3,3-ioene, N-(cycloalkyl)alkylamines and poliglusam, insoluble quaternized polystyrenes, saponins and mixtures thereof. Suitable inorganic cholesterol sequestrants include bismuth salicylate plus montmorillonite clay, aluminum hydroxide and calcium carbonate antacids.

Another embodiment of the present invention include activators or agonists of PPAR. The activators act as agonists for the peroxisome proliferator-activated receptors. Three subtypes of PPAR have been identified, and these are designated as peroxisome proliferator-activated receptor alpha (PPARα), peroxisome proliferator-activated receptor gamma (PPARγ) and peroxisome proliferator-activated receptor delta (PPARδ). It should be noted that PPARδ is also referred to in the literature as PPARβ and as NUC1, and each of these names refers to the same receptor.

PPARα regulates the metabolism of lipids. PPARα is activated by fibrates and a number of medium and long-chain fatty acids, and it is involved in stimulating β-oxidation of fatty acids. The PPARγ receptor subtypes are involved in activating the program of adipocyte differentiation and are not involved in stimulating peroxisome proliferation in the liver. PPARδ has been identified as being useful in increasing high density lipoprotein (HDL) levels in humans. See, e.g., WO 97/28149.

PPARα activator compounds are useful for, among other things, lowering triglycerides, moderately lowering LDL levels and increasing HDL levels. Useful examples of PPARα activators include fibrates.

Non-limiting examples of suitable fibric acid derivatives (“fibrates”) include clofibrate (such as ethyl 2-(p-chlorophenoxy)-2-methyl-propionate, for example ATROMID-S® Capsules which are commercially available from Wyeth-Ayerst); gemfibrozil (such as 5-(2,5-dimethylphenoxy)-2,2-dimethylpentanoic acid, for example LOPID® tablets which are commercially available from Pfizer); ciprofibrate (C.A.S. Registry No. 52214-84-3, see U.S. Pat. No. 3,948,973 which is incorporated herein by reference); bezafibrate (C.A.S. Registry No. 41859-67-0, see U.S. Pat. No. 3,781,328 which is incorporated herein by reference); clinofibrate (C.A.S. Registry No. 30299-08-2, see U.S. Pat. No. 3,716,583 which is incorporated herein by reference); binifibrate (C.A.S. Registry No. 69047-39-8, see BE 884722 which is incorporated herein by reference); lifibrol (C.A.S. Registry No. 96609-16-4); fenofibrate (such as TRICOR® micronized fenofibrate (2-[4-(4-chlorobenzoyl)phenoxy]-2-methyl-propanoic acid, 1-methylethyl ester) which is commercially available from Abbott Laboratories or LIPANTHYL® micronized fenofibrate which is commercially available from Labortoire Founier, France) and mixtures thereof. These compounds can be used in a variety of forms, including but not limited to acid form, salt form, racemates, enantiomers, zwitterions and tautomers.

Other examples of PPARα activators useful in the practice of the present invention include suitable fluorophenyl compounds as disclosed in U.S. Pat. No. 6,028,109 which is incorporated herein by reference; certain substituted phenylpropionic compounds as disclosed in WO 00/75103 which is incorporated herein by reference; and PPARα activator compounds as disclosed in WO 98/43081 which is incorporated herein by reference.

Non-limiting examples of suitable PPARγ activators include derivatives of glitazones or thiazolidinediones, such as, troglitazone; rosiglitazone (such as AVANDIA® rosiglitazone maleate (-5-[[4-[2-(methyl-2-pyridinylamino)ethoxy]phenyl]methyl]-2,4-thiazolidinedione-2-butenedioate) commercially available from SmithKline Beecham) and pioglitazone (such as ACTOS™ pioglitazone hydrochloride (5-[[4-[2-(5-ethyl-2-pyridinyl)ethoxy]phenyl]methyl]-2,4-]thiazolidinedione monohydrochloride) commercially available from Takeda Pharmaceuticals). Other useful thiazolidinediones include ciglitazone, englitazone, darglitazone and BRL 49653 as disclosed in WO 98/05331 which is incorporated herein by reference; PPARγ activator compounds disclosed in WO 00/76488 which is incorporated herein by reference; and PPARy activator compounds disclosed in U.S. Pat. No. 5,994,554 which is incorporated herein by reference.

Other useful PPARγ activator compounds include certain acetylphenols as disclosed in U.S. Pat. No. 5,859,051 which is incorporated herein by reference; certain quinoline phenyl compounds as disclosed in WO 99/20275 which is incorporated herein by reference; aryl compounds as disclosed by WO 99/38845 which is incorporated herein by reference; certain 1,4-disubstituted phenyl compounds as disclosed in WO 00/63161; certain aryl compounds as disclosed in WO 01/00579 which is incorporated herein by reference; benzoic acid compounds as disclosed in WO 01/12612 & WO 01/12187 which are incorporated herein by reference; and substituted 4-hydroxy-phenylalconic acid compounds as disclosed in WO 97/31907 which is incorporated herein by reference.

PPARδ compounds are useful for, among other things, lowering triglyceride levels or raising HDL levels. Non-limiting examples of PPARδ activators include suitable thiazole and oxazole derivatives, such as C.A.S. Registry No. 317318-32-4, as disclosed in WO 01/00603 which is incorporated herein by reference); certain fluoro, chloro or thio phenoxy phenylacetic acids as disclosed in WO 97/28149 which is incorporated herein by reference; suitable non-β-oxidizable fatty acid analogues as disclosed in U.S. Pat. No. 5,093,365 which is incorporated herein by reference; and PPARδ compounds as disclosed in WO 99/04815 which is incorporated herein by reference.

Moreover, compounds that have multiple functionality for activating various combinations of PPARα, PPARγ and PPARδ are also useful with the practice of the present invention. Non-limiting examples include certain substituted aryl compounds as disclosed in U.S. Pat. No. 6,248,781; WO 00/23416; WO 00/23415; WO 00/23425; WO 00/23445; WO 00/23451; and WO 00/63153, all of which are incorporated herein by reference, are described as being useful PPARα and/or PPARγ activator compounds. Other non-limiting examples of useful PPARα and/or PPARγ activator compounds include activator compounds as disclosed in WO 97/25042 which is incorporated herein by reference; activator compounds as disclosed in WO 00/63190 which is incorporated herein by reference; activator compounds as disclosed in WO 01/21181 which is incorporated herein by reference; biaryl-oxa(thia)zole compounds as disclosed in WO 01/16120 which is incorporated herein by reference; compounds as disclosed in WO 00/63196 and WO 00/63209 which are incorporated herein by reference; substituted 5-aryl-2,4-thiazolidinediones compounds as disclosed in U.S. Pat. No. 6,008,237 which is incorporated herein by reference; arylthiazolidinedione and aryloxazolidinedione compounds as disclosed in WO 00/78312 and WO 00/78313G which are incorporated herein by reference; GW2331 or (2-(4-[difluorophenyl]-1heptylureido)ethyl]phenoxy)-2-methylbutyric compounds as disclosed in WO 98/05331 which is incorporated herein by reference; aryl compounds as disclosed in U.S. Pat. No. 6,166,049 which is incorporated herein by reference; oxazole compounds as disclosed in WO 01/17994 which is incorporated herein by reference; and dithiolane compounds as disclosed in WO 01/25225 and WO 01/25226 which are incorporated herein by reference.

Other useful PPAR activator compounds include substituted benzylthiazolidine-2,4-dione compounds as disclosed in WO 01/14349, WO 01/14350 and WO/01/04351 which are incorporated herein by reference; mercaptocarboxylic compounds as disclosed in WO 00/50392 which is incorporated herein by reference; ascofuranone compounds as disclosed in WO 00/53563 which is incorporated herein by reference; carboxylic compounds as disclosed in WO 99/46232 which is incorporated herein by reference; compounds as disclosed in WO 99/12534 which is incorporated herein by reference; benzene compounds as disclosed in WO 99/15520 which is incorporated herein by reference; o-anisamide compounds as disclosed in WO 01/21578 which is incorporated herein by reference; and PPAR activator compounds as disclosed in WO 01/40192 which is incorporated herein by reference.

The peroxisome proliferator-activated receptor(s) activator(s) are administered in a therapeutically effective amount to treat the specified condition, for example in a daily dose preferably ranging from about 50 to about 3000 mg per day, and more preferably about 50 to about 2000 mg per day, given in a single dose or 2-4 divided doses. The exact dose, however, is determined by the attending clinician and is dependent on such factors as the potency of the compound administered, the age, weight, condition and response of the patient.

In an alternative embodiment, the present invention includes the use of one or more IBAT inhibitors or ASBT inhibitors. The IBAT inhibitors can inhibit bile acid transport to reduce LDL cholesterol levels. Non-limiting examples of suitable IBAT inhibitors include benzothiepines such as therapeutic compounds comprising a 2,3,4,5-tetrahydro-1-benzothiepine 1,1-dioxide structure such as are disclosed in PCT Patent Application WO 00/38727 which is incorporated herein by reference.

Generally, a total daily dosage of IBAT inhibitor(s) can range from about 0.01 to about 1000 mg/day, and preferably about 0.1 to about 50 mg/day in single or 2-4 divided doses.

In another alternative embodiment, the methods of the present invention can further comprise nicotinic acid (niacin) and/or nicotinic acid receptor (“NAR”) agonists as lipid lowering agents.

As used herein, “nicotinic acid receptor agonist” means any compound comprising that will act as an agonist to the nicotinic acid receptor. Compounds include those that have a pyridine-3-carboxylate structure or a pyrazine-2-carboxylate structure, including acid forms, salts, esters, zwitterions and tautomers, where available. Examples of nicotinic acid receptor agonists include niceritrol, nicofuranose and acipimox (5-methyl pyrazine-2-carboxylic acid 4-oxide). Nicotinic acid and NAR agonists inhibit hepatic production of VLDL and its metabolite LDL and increases HDL and apo A-1 levels. An example of a suitable nicotinic acid product is NIASPAN® (niacin extended-release tablets) which are available from Kos.

Generally, a total daily dosage of nicotinic acid can range from about 500 to about 10,000 mg/day, preferably about 1000 to about 8000 mg/day, and more preferably about 3000 to about 6000 mg/day in single or divided doses. Generally, the total daily dosage of a NAR agonist can range from about 1 to about 100 mg/day/

In another alternative embodiment, the methods of the present invention can further comprise one or more ACAT inhibitors as lipid lowering agents. ACAT inhibitors reduce LDL and VLDL levels ACAT is an enzyme responsible for esterifying excess intracellular cholesterol and may reduce the synthesis of VLDL, which is a product of cholesterol esterification, and overproduction of apo B-100-containing lipoproteins.

Non-limiting examples of useful ACAT inhibitors include avasimibe ([[2,4,6-tris(1-methylethyl)phenyl]acetyl]sulfamic acid, 2,6-bis(1-methylethyl)phenyl ester, formerly known as CI-1011), HL-004, lecimibide (DuP-128) and CL-277082 (N-(2,4-difluorophenyl)-N-[[4-(2,2-dimethylpropyl)phenyl]methyl]-N-heptylurea). See P. Chang et al., “Current, New and Future Treatments in Dyslipidaemia and Atherosclerosis”, Drugs 2000 July;60(1); 55-93, which is incorporated by reference herein.

Generally, a total daily dosage of ACAT inhibitor(s) can range from about 0.1 to about 1000 mg/day in single or 2-4 divided doses.

In another alternative embodiment, the compositions used in the methods of the present invention can further comprise one or more Cholesteryl Ester Transfer Protein (“CETP”) Inhibitors coadministered with or in combination with the compound(s) of Formulae I-X discussed above. CETP is responsible for the exchange or transfer of cholesteryl ester carrying HDL and triglycerides in VLDL.

Non-limiting examples of suitable CETP inhibitors are disclosed in PCT Patent Application No. WO 00/38721 and U.S. Pat. No. 6,147,090, which are incorporated herein by reference. Pancreatic cholesteryl ester hydrolase (PCEH) inhibitors such as WAY-121898 also can be coadministered with or in combination with the fibric acid derivative(s) and sterol absorption inhibitor(s) discussed above.

Generally, a total daily dosage of CETP inhibitor(s) can range from about 0.01 to about 1000 mg/day, and preferably about 0.5 to about 20 mg/kg body weight/day in single or divided doses.

In another alternative embodiment, the methods of the present invention can further comprise probucol or derivatives thereof (such as AGI-1067 and other derivatives disclosed in U.S. Pat. Nos. 6,121,319 and 6,147,250), which can reduce LDL and HDL levels, as cholesterol lowering agents.

Generally, a total daily dosage of probucol or derivatives thereof can range from about 10 to about 2000 mg/day, and preferably about 500 to about 1500 mg/day in single or 2-4 divided doses.

In another alternative embodiment, the methods of the present invention can further comprise one or more low-density lipoprotein (LDL) receptor activators, as lipid lowering agents. Non-limiting examples of suitable LDL-receptor activators include HOE-402, an imidazolidinyl-pyrimidine derivative that directly stimulates LDL receptor activity. See M. Huettinger et al., “Hypolipidemic activity of HOE-402 is Mediated by Stimulation of the LDL Receptor Pathway”, Arterioscler. Thromb. 1993; 13:1005-12.

Generally, a total daily dosage of LDL receptor activator(s) can range from about 1 to about 1000 mg/day in single or 2-4 divided doses.

In another alternative embodiment, the methods of the present invention can further comprise fish oil, which contains Omega 3 fatty acids (3-PUFA), which can reduce VLDL and triglyceride levels, as a lipid lowering agent. Generally, a total daily dosage of fish oil or Omega 3 fatty acids can range from about 1 to about 30 grams per day in single or 2-4 divided doses.

In another alternative embodiment, the methods of the present invention can further comprise natural water soluble fibers, such as psyllium, guar, oat and pectin, which can reduce cholesterol levels. Generally, a total daily dosage of natural water soluble fibers can range from about 0.1 to about 10 grams per day in single or 2-4 divided doses.

In another alternative embodiment, methods of the present invention can further comprise plant sterols, plant stanols and/or fatty acid esters of plant stanols, such as sitostanol ester used in BENECOL® margarine, which can reduce cholesterol levels. Generally, a total daily dosage of plant sterols, plant stanols and/or fatty acid esters of plant stanols can range from about 0.5 to about 20 grams per day in single or 2-4 divided doses.

As discussed above, the compositions, therapeutic combinations and methods of the present invention may comprise at least one H₃ receptor antagonist/inverse agonist. In one embodiment, the H₃ receptor antagonist inverse agonist can be one of the imidazole type, such as those described in WO 95/14007 and WO 99/21405, each herein incorporated by reference.

In yet another embodiment of present invention provides for compositions, therapeutic combinations and methods of the present invention, wherein at least one H₃ receptor antagonist/inverse agonist is a compound of the formula: 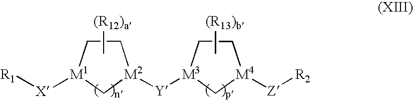
or a pharmaceutically acceptable salt or solvate thereof, wherein:

(1) R₁ is selected from:
(a) aryl;
(b) heteroaryl;
(c) heterocycloalkyl
(d) alkyl;
(e) cycloalkyl; or
(f) alkylaryl;
   wherein said R¹ groups are optionally substituted with 1 to 4 substituents independently selected from:
(1) halogen (e.g., Br, F, or Cl, preferably F or Cl);
(2) hydroxyl (i.e., —OH);
(3) lower alkoxy (e.g., C₁ to C₆ alkoxy, preferably C₁ to C₄ alkoxy, most preferably C₁ to C₂ alkoxy, more preferably methoxy);
(4) —CF₃;
(5) CF₃O—;
(6) —NR₄R₅;
(7) phenyl;
(8) —NO₂,
(9) —CO₂R₄;
(10) —CON(R₄)₂ wherein each R₄ is the same or different;
(11) —S(O)_{m′}N(R₂₀)₂ wherein each R₂₀ is the same or different H or alkyl group, preferably C₁ to C₄ alkyl, most preferably C₁-C₂ alkyl, and more preferably methyl;
(12) —CN; or
(13) alkyl; or

(2) R₁ and X′ taken together form a group selected from:

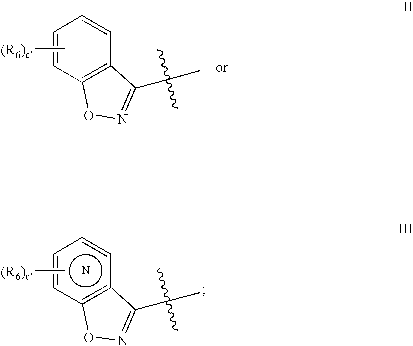

(3) X′ is selected from: ═C(O), ═C(NOR₃), ═C(NNR₄R₅),

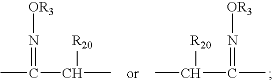

(4) M¹ is carbon;

(5) M² is selected from C or N;

(6) M³ and M⁴ are independently selected from C or N;

(7) Y′ is selected from: is —CH₂—, ═C(O), ═C(NOR₂₀) (wherein R₂₀ is as defined above), or ═C(S);

(8) Z′ is a C₁-C₆ alkyl group;

(9) R₂ is a five or six-membered heteroaryl ring, said six-membered heteroaryl ring comprising 1 or 2 nitrogen atoms with the remaining ring atoms being carbon, and said five-membered heteroaryl ring containing 1 or 2 heteroatoms selected from: nitrogen, oxygen, or sulfur with the remaining ring atoms being carbon; said five or six membered heteroaryl rings being optionally substituted with 1 to 3 substituents independently selected from: halogen, hydroxyl, lower alkyl, lower alkoxy, —CF₃, CF₃O—, —NR₄R₅, phenyl, —NO₂, —CO₂R₄, —CON(R₄)₂ wherein each R₄ is the same or different, —CH2NR₄R₅, —(N)C(NR₄R₅)₂, or —CN;

(10) R₃ is selected from:
(a) hydrogen;
(b) C₁-C₆ alkyl;
(c) aryl;
(d) heteroaryl;
(e) heterocycloalkyl;
(f) arylalkyl (e.g., aryl(C₁ to C₄)alkyl, e.g., —(CH₂)_{w′}aryl wherein w′ is 1 to 4, preferably 1 or 2, and most preferably 1, such as, for example —CH₂phenyl or —CH₂substituted phenyl);
(g) —(CH₂)_{e′}—C(O)N(R₄)₂ wherein each R₄ is the same or different,
(h) —(CH₂)_{e′}—C(O)OR₄;
(i) —(CH₂)_{e′}—C(O)R₃₀ wherein R₃₀ is a heterocycloalkyl group, such as, for example, morpholinyl, piperidinyl, piperazinyl or pyrrolidinyl, including 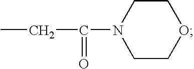
(j) —CF₃; or
(k) —CH₂CF₃;
   wherein said aryl, heteroaryl, heterocycloalkyl, and the aryl portion of said arylalkyl are optionally substituted with 1 to 3 (preferably 1) substituents selected from: halogen (e.g., F or Cl), —OH, —OCF₃, —CF₃, —CN, —N(R₄₅)₂, —CO₂R₄₅, or —C(O)N(R₄₅)₂, wherein each R₄₅ is independently selected from: H, alkyl, alkylaryl, or alkylaryl wherein said aryl moiety is substituted with 1 to 3 substituents independently selected from —CF₃, —OH, halogen, alkyl, —NO₂, or —CN;

(11) R₄ is selected from: hydrogen, C₁-C₆ alkyl, aryl, alkylaryl, said aryl and alkylaryl groups being optionally substituted with 1 to 3 substituents selected from: halogen, —CF₃, —OCF₃, —OH, —N(R₄₅)₂, —CO₂R₄₅, —C(O)N(R₄₅)₂, or —CN; wherein R₄₅ is as defined above;

(12) R₅ is selected from: hydrogen, C₁-C₆ alkyl, —C(O)R₄, —C(O)₂R₄, or —C(O)N(R₄)₂ wherein each R₄ is independently selected, and R₄ is as defined above;

(13) or R₄ and R₅ taken together with the nitrogen atom to which they are bound forms a five or six membered heterocycloalkyl ring (e.g., morpholine);

(14) R₆ is selected from: alkyl, aryl, alkylaryl, halogen, hydroxyl, lower alkoxy, —CF₃, CF₃O—, —NR₄R₅, phenyl, —NO₂, —CO₂R₅, —CON(R₄)₂ wherein each R₄ is the same or different, or —CN;

(15) R₁₂ is selected from: alkyl, hydroxyl, alkoxy, or fluoro;

(16) R₁₃ is selected from: alkyl, hydroxyl, alkoxy, or fluoro;

(17) a′ (subscript for R₁₂) is 0 to 2;

(18) b′ (subscript for R₁₂) is 0 to 2;

(19) c′ (subscript for R₆) is 0 to 2;

(20) e′ is o to 5;

(21) m′ is 1 or 2;

(22) n′ is 1, 2 or 3; and

(23) p′ is 1, 2 or 3, with the proviso that when M³ and M⁴ are both nitrogen, then p′ is 2 or 3 (i.e., p′ is not 1 when M³ and M² are both nitrogen) is present in the therapeutic combinations.

More preferred definitions for the compounds of formula XIII are as follows:

R₁ is preferably selected from:
(A) aryl (most preferably phenyl);
(B) substituted aryl (e.g., substituted phenyl), wherein the substituents on said substituted aryl are most preferably selected from: (1) halo (e.g., monohalo or dihalo), more preferably chloro or fluoro, even more preferably monochloro, dichloro, monofluoro or difluoro; or (2) alkyl, more preferably unbranched (i.e., straight chain, e.g., methyl) alkyl, even more preferably substituted alkyl, still more preferably alkyl substituted with halo (e.g., 1, 2 or 3 halo atoms, such as Cl or F), even still more preferably alkyl substituted with fluoro atoms, yet still more preferably trifluromethyl;
(C) heteroaryl, most preferably a five or six membered heteroaryl ring, more preferably a six membered heteroaryl ring, and still more preferably pyridyl, examples of heteroaryl rings include pyridyl, thienyl, pyrimidinyl, thiazolyl or pyridyl N-Oxide, most preferred heteroaryl rings are exemplified by 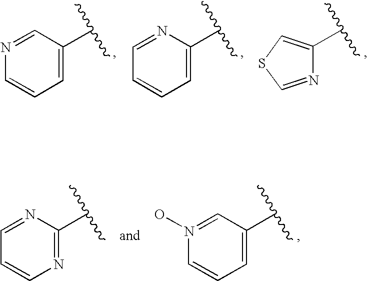
   wherein 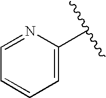
   is preferred more;
(D) substituted heteroaryl, most preferably halo or alkyl substituted heteroaryl (e.g., halopyridyl (e.g., fluoropyridyl) and alkylthiazolyl), more preferably substituted heteroaryl wherein the substituents are independently selected from the same or different alkyl groups (even more preferably one straight chain alkyl group, e.g., methyl), still more preferably alkyl substituted thiazolyl, and even more preferably 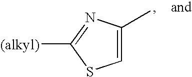
   yet even more preferably 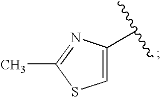
(E) when R₁ is taken together with X′, then the moiety is 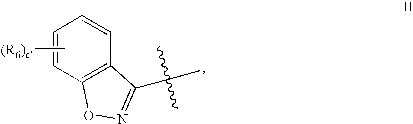
   wherein c′ is most preferably 0 or 1, and when c′ is 1 then R₆ is most preferably halo, and when c′ is 1 then R₆ is more preferably fluoro.

X′ is preferably ═C(NOR₃) wherein R₃ is preferably selected from H, alkyl or halo substituted alkyl (e.g., fluoro substituted alkyl, such as —CH₂CF₃), most preferably alkyl, more preferably methyl or ethyl, and still more preferably methyl.

Preferably M² is nitrogen.

n′ is preferably 2.

a′ is preferably 0 or 1, and most preferably 0.

b′ is preferably 0 or 1, and most preferably 0.

c′ is preferably 0 or 1, and most preferably 0, and when c is 1 then R₆ is preferably halo, and when c is 1 R₆ is most preferably fluoro.

e′ is preferably 1-5.

Y′ is preferably ═C(O) (i.e., ═C═O).

M³ and M⁴ are preferably selected such that: (1) one is carbon and the other is nitrogen, or (2) both are nitrogen, with M³ most preferably being carbon.

p′ is preferably 2.

Z′ is preferably C₁ to C₃ alkyl, and most preferably

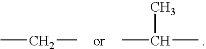

R₂ is preferably a six membered heteroaryl ring, most preferably pyridyl, substituted pyridyl, pyrimidinyl or substituted pyrimidinyl, more preferably pyridyl, pyridyl substituted with —NR₄R₅, pyrimidinyl or pyrimidinyl substituted with —NR₄R₅, still more preferably pyridyl, pyridyl substituted with —NH₂ (i.e., R₄ and R₅ are H), pyrimidinyl or pyrimidinyl substituted with —NH₂ (i.e., R₄ and R₅ are H), and even more preferably 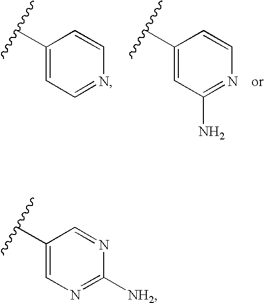
and still even more preferably

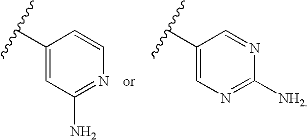

R₃ is preferably H or alkyl, most preferably H or methyl.

R₄ is preferably H or lower alkyl, most preferably H or methyl, and more preferably H.

R₅ is preferably H, C₁ to C₆alkyl or —C(O)R₄, most preferably H or methyl, and more preferably H.

R₁₂ is preferably alkyl, hydroxyl or fluoro, and most preferably H.

R₁₃ is preferably alkyl, hydroxyl or fluoro, and most preferably H.

Methods for making compounds of formula XIII are well known to those skilled in the art. Non-limiting examples of suitable methods are disclosed in U.S. Pat. No. 6,720,328 B1, herein incorporated by reference.

Examples of compounds of formula XIII that are useful in this invention are represented by the following formulae:

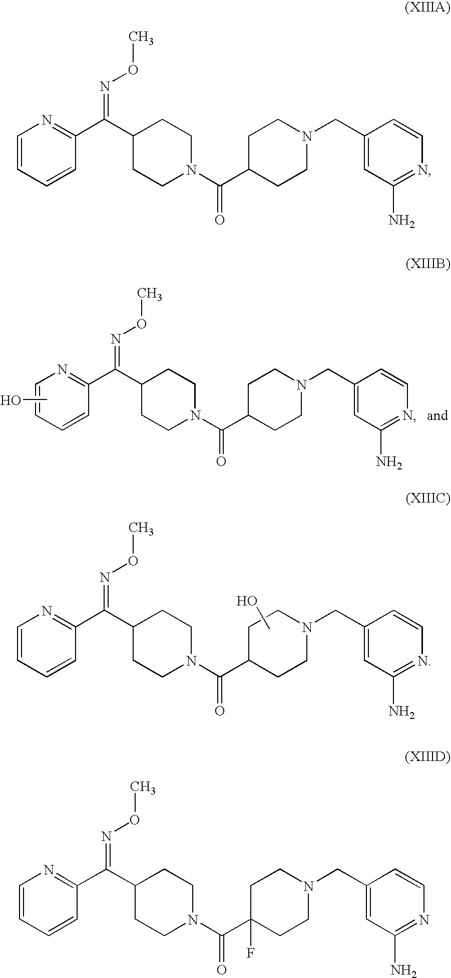

In yet another embodiment, this invention provides for compositions, therapeutic combinations and methods of the present invention wherein at least one H₃ receptor antagonist/inverse agonist is a compound of the formula: 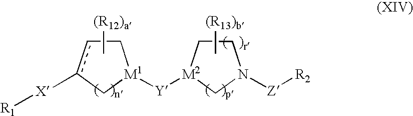
or a pharmaceutically acceptable salt or solvate thereof, wherein:

the dotted line represents an optional double bond;

a′ is 0 to 2;

b′ is 0 to 2;

n′ is 1, 2 or 3;

p′ is 1, 2 or 3;

r′ is 0, 1, 2, or 3;

with the provisos that when M² is N, p′ is not 1; and that when r′ is 0, M² is C(R₃); and that the sum of p′ and r′ is 1 to 4;

M¹ is C(R₃) or N;

M² is C(R₃) or N;

X′ is a bond or C₁-C₆ alkylene;

Y′ is —C(O)—, —C(S), —(CH₂)_{q′}—, —NR₄C(O)—, —C(O)NR₄—, —C(O)CH₂—, —SO₂—, —N(R₄)—, —NH—C(═N—CN)— or —C(═N—CN)—N H—; with the provisos that when M¹ is N, Y′ is not —NR₄C(O)— or —NH—C(═N—CN)—; when M² is N, Y′ is not —C(O)NR₄— or —C(═N—CN)—NH—; and when Y′ is —N(R₄)—, M¹ is CH and M₂ is C(R₃);

q′ is 1 to 5, provided that when both M¹ and M² are N, q′ is 2 to 5;

Z′ is a bond, C₁-C₆ alkylene, C₁-C₆ alkenylene, —C(O)—, —CH(CN)—, —SO₂— or —CH₂C(O)NR₄—;

R₁ is

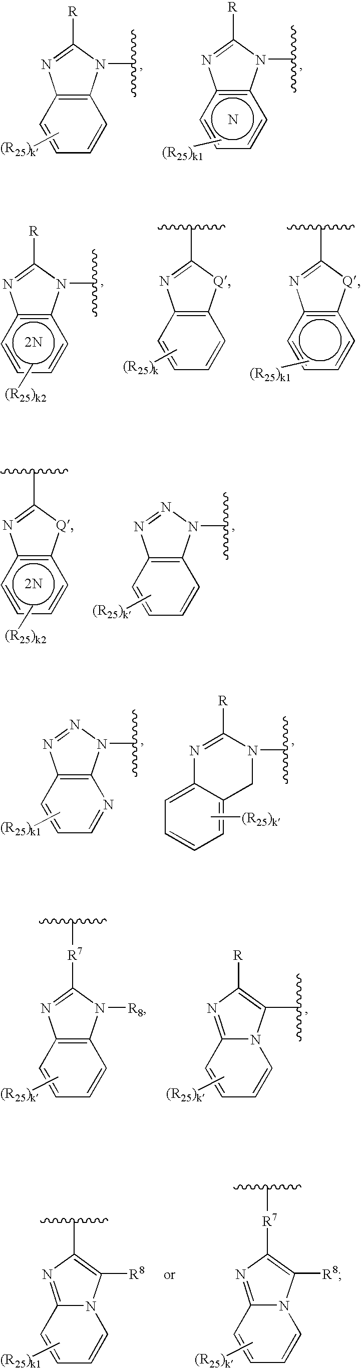

Q′ is —N(R₈)—, —S— or —O—;

k′ is 0, 1, 2, 3 or 4;

k1 is 0, 1, 2 or 3;

k2 is 0, 1 or 2;

R is H, C₁-C₆ alkyl, halo(C₁-C₆)alkyl-, C₁-C₆ alkoxy, (C₁-C₆)alkoxy-(C₁-C₆)alkyl-, (C₁-C₆)alkoxy-(C₁-C₆)alkoxy, (C₁-C₆)alkoxy-(C₁-C₆)alkyl-SO₀₋₂, R₃₂-aryl(C₁-C₆)alkoxy-, R₃₂-aryl(C₁-C₆)alkyl-, R₃₂-aryl, R₃₂-aryloxy, R₃₂-heteroaryl, (C₃-C₆)cycloalkyl, (C₃-C₆)cycloalkyl-(C₁-C₆)alkyl, (C₃-C₆)cycloalkyl-(C₁-C₆)alkoxy, (C₃-C₆)cycloalkyl-oxy-, R₃₇-heterocycloalkyl, R₃₇-heterocycloalkyl-oxy-, R₃₇-heterocycloalkyl-(C₁-C₆)alkoxy, N(R₃₀)(R₃₁)-(C₁-C₆)alkyl-, —N(R₃₀)(R₃₁), —NH—(C₁-C₆)alkyl-O—(C₁-C₆)alkyl, —NHC(O)NH(R₂₉); R₂₉—S(O)₀₋₂—, halo(C₁-C₆)alkyl-S(O)₀₋₂—, N(R₃₀)(R₃₁)—(C₁-C₆)alkyl-S(O)₀₋₂— or benzoyl;

R₈ is H, C₁-C₆ alkyl, halo(C₁-C₆)alkyl-, (C₁-C₆)alkoxy-(C₁-C₆)alkyl-, R₃₂-aryl(C₁-C₆)alkyl-, R₃₂-aryl, R₃₂-heteroaryl, (C₃-C₆)cycloalkyl, (C₃-C₆)cycloalkyl-(C₁-C₆)alkyl, R₃₇-heterocycloalkyl, N(R₃₀)(R₃₁)—(C₁-C₆)alkyl-, R₂₉—S(O)₂—, halo(C₁-C₆)alkyl-S(O)₂—, R₂₉—S(O)₀₋₁—(C₂-C₆)alkyl-, halo(C₁-C₆)alkyl-S(O)₀₋₁—(C₂-C₆)alkyl-;

R₃ is a six-membered heteroaryl ring having 1 or 2 heteroatoms independently selected from N or N—O, with the remaining ring atoms being carbon; a five-membered heteroaryl ring having 1, 2, 3 or 4 heteroatoms independently selected from N, O or S, with the remaining ring atoms being carbon; R₃₂-quinolyl; R₃₂-aryl; heterocycloalkyl; (C₃-C₆)cycloalkyl; C₁-C₆ alkyl; hydrogen; thianaphthenyl; 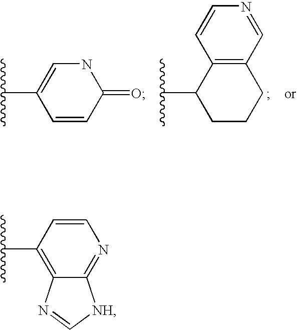
wherein said six-membered heteroaryl ring or said five-membered heteroaryl ring is optionally substituted by R₆;

R₃ is H, halogen, C₁-C₆ alkyl, —OH, (C₁-C₆)alkoxy or —NHSO₂—(C₁-C₆)alkyl;

R₄ is independently selected from the group consisting of hydrogen, C₁-C₆ alkyl, C₃-C₆ cycloalkyl, (C₃-C₆)cycloalkyl(C₁-C₆)alkyl, R₃₃-aryl, R₃₃-aryl(C₁-C₆)alkyl, and R₃₂-heteroaryl;

R₅ is hydrogen, C₁-C₆ alkyl, —C(O)R₂₀, —C(O)₂R₂₀, —C(O)N(R₂₀)₂, (C₁-C₆)alkyl-SO₂—, or (C₁-C₆)alkyl-SO₂—NH—;

or R₄ and R₅, together with the nitrogen to which they are attached, form an azetidinyl, pyrrolidinyl, piperidinyl, piperazinyl or morpholinyl ring;

R₆ is 1 to 3 substituents independently selected from the group consisting of —OH, halogen, C₁-C₆ alkyl-, C₁-C₆ alkoxy, C₁-C₆ alkylthio, —CF₃, —NR₄R₅, —CH₂—NR₄R₅, —NHSO₂R₂₂, —N(SO₂R₂₂)₂, phenyl, R₃₃-phenyl, NO₂, —CO₂R₄, —CON(R₄)₂,

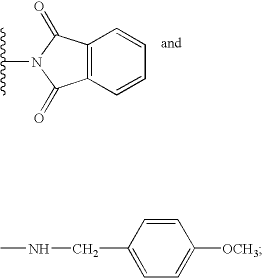

R₇ is —N(R₂₉)—, —O— or —S(O)₀₋₂—;

R₁₂ is independently selected from the group consisting of C₁-C₆ alkyl, hydroxyl, C₁-C₆ alkoxy, or fluoro, provided that when R₁₂ is hydroxy or fluoro, then R₁₂ is not bound to a carbon adjacent to a nitrogen; or two R₁₂ substituents form a C₁ to C₂ alkyl bridge from one ring carbon to another non-adjacent ring carbon; or R₁₂ is ═O;

R₁₃ is independently selected from the group consisting of C₁-C₆ alkyl, hydroxyl, C₁-C₆ alkoxy, or fluoro, provided that when R₁₃ is hydroxy or fluoro then R₁₃ is not bound to a carbon adjacent to a nitrogen; or two R₁₃ substituents form a C₁ to C₂ alkyl bridge from one ring carbon to another non-adjacent ring carbon; or R₁₃ is ═O;

R₂ is independently selected from the group consisting of hydrogen, C₁-C₆ alkyl, or aryl, wherein said aryl group is optionally substituted with from 1 to 3 groups independently selected from halogen, —CF₃, —OCF₃, hydroxyl, or methoxy; or when two R₂₀ groups are present, said two R₂₀ groups taken together with the nitrogen to which they are bound can form a five or six membered heterocyclic ring;

R₂₂ is C₁-C₆ alkyl, R₃₄-aryl or heterocycloalkyl;

R₂₄ is H, C₁-C₆ alkyl, —SO₂R₂ or R₃₄-aryl;

R₂₅ is independently selected from the group consisting of C₁-C₆ alkyl, halogen, —CN, —NO₂, —CF₃, —OH, C₁-C₆ alkoxy, (C₁-C₆)alkyl-C(O)—, aryl-C(O)—, —C(O)OR₂₉, —N(R₄)(R₅), N(R₄)(R₅)—C(O)—, N(R₄)(R₅)—S(O)₁₋₂—, R₂₂—S(O)₀₋₂—, halo-(C₁-C₆)alkyl- or halo-(C₁-C₆)alkoxy-(C₁-C₆)alkyl-;

R₂₉ is H, C₁-C₆ alkyl, C₃-C₆ cycloalkyl, R₃₅-aryl or R₃₅-aryl(C₁-C₆)alkyl-;

R₃₀ is H, C₁-C₆ alkyl-, R₃₅-aryl or R₃₅-aryl(C₁-C₆)alkyl-;

R₃₁ is H, C₁-C₆ alkyl-, R₃₅-aryl, R₃₅-aryl(C₁-C₆)alkyl-, R₃₅-heteroaryl, (C₁-C₆)alkyl-C(O)—, R₃₅-aryl-C(O)—, N(R₄)(R₅)—C(O)—, (C₁-C₆)alkyl-S(O)₂— or R₃₅-aryl-S(O)₂—;

or R₃₀ and R₃₁ together are —(CH₂)₄₋₅—, —CH₂)₂—O—(CH₂)₂— or —(CH₂)₂—N(R₃₈)—(CH₂)₂— and form a ring with the nitrogen to which they are attached;

R₃₂ is 1 to 3 substituents independently selected from the group consisting of H, —OH, halogen, C₁-C₆ alkyl, C₁-C₆ alkoxy, R₃₅-aryl-O—, —SR₂₂, —CF₃, —OCF₃, —OCHF₂, —NR₃₉R₄₀, phenyl, R₃₃-phenyl, NO₂, —CO₂R₃₉, —CON(R₃₉)₂, —S(O)₂R₂₂, —S(O)₂N(R₂₀)₂, —N(R₂₄)S(O)₂R₂₂, —CN, hydroxy-(C₁-C₆)alkyl-, —OCH₂CH₂OR₂₂, and R₃₅-aryl(C₁-C₆)alkyl-O—, or two R₃₂ groups on adjacent carbon atoms together form a —OCH₂O— or —O(CH₂)₂O— group;

R₃₃ is 1 to 3 substituents independently selected from the group consisting of C₁-C₆ alkyl, halogen, —CN, —NO₂, —CF₃, —OCF₃, —OCHF₂ and —O—(C₁-C₆)alkyl;

R₃₄ is 1 to 3 substituents independently selected from the group consisting of H, halogen, —CF₃, —OCF₃, —OH and —OCH₃;

R₃₅ is 1 to 3 substituents independently selected from hydrogen, halo, C₁-C₆ alkyl, hydroxy, C₁-C₆ alkoxy, phenoxy, —CF₃, —N(R₃₆)₂, —COOR₂₀ and —NO₂;

R₃₆ is independently selected form the group consisting of H and C₁-C₆ alkyl;

R₃₇ is 1 to 3 substituents independently selected from hydrogen, halo, C₁-C₆ alkyl, hydroxy, C₁-C₆ alkoxy, phenoxy, —CF₃, —N(R₃₆)₂, —COOR₂₀, —C(O)N(R₂₉)₂ and —NO₂, or R₃₇ is one or two ═O groups;

R₃₈ is H, C₁-C₆ alkyl, R₃₅-aryl, R₃₅-aryl(C₁-C₆)alkyl-, (C₁-C₆)alkyl-SO₂ or halo(C₁-C₆)alkyl-SO₂—;

R₃₉ is independently selected from the group consisting of hydrogen, C₁-C₆ alkyl, C₃-C₆ cycloalkyl, (C₃-C₆)cycloalkyl(C₁-C₆)alkyl, R₃₃-aryl, R₃₃-aryl(C₁-C₆)alkyl, and R₃₂-heteroaryl; and

R₄₀ is hydrogen, C₁-C₆ alkyl, —C(O)R₂₀, —C(O)₂R₂₀, —C(O)N(R₂₀)₂, (C₁-C₆)alkyl-SO₂—, or (C₁-C₆)alkyl-SO₂—NH—;

or R₃₉ and R₄₀, together with the nitrogen to which they are attached, form an azetidinyl, pyrrolidinyl, piperidinyl, piperazinyl or morpholinyl ring.

Methods for making compounds of formula XIV are well known to those skilled in the art. Non-limiting examples of suitable methods are disclosed in US Publication No US 2004/0097483A1, herein incorporated by reference.

Another embodiment, this invention provides for compositions, therapeutic combinations and methods of the present inventions wherein the H₃ receptor antagonist/inverse agonist is a compound of the formula: 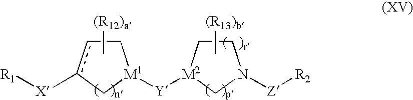
or a pharmaceutically acceptable salt or solvate thereof, wherein:

a′ is 0 to 3;

b′ is 0 to 3;

n′ is 1, 2 or 3;

p′ is 1, 2 or 3;

r′ is 0, 1, 2, or 3;

X′ is a bond or C₁-C₆ alkylene;

M¹ is CH or N;

M² is C(R₃) or N;

with the provisos that when M² is N, p′ is not 1; and that when r′ is 0, M² is C(R₃); and that the sum of p′ and r′ is 1 to 4;

Y′ is —C(═O)—, —C(═S)—, —(CH₂)_{q′}—, —NR₄C(═O)—, —C(═O)NR₄—, —C(═O)CH₂—, —SO₁₋₂—, —C(═N—CN)—NH— or —NH—C(═N—CN)—; with the provisos that when M¹ is N, Y′ is not —NR₄C(═O)— or —NH—C(═N—CN)—; and when M² is N, Y′ is not —C(═O)NR₄— or —C(═N—CN)—NH—;

q′ is 1 to 5, provided that when M¹ and M² are both N, q′ is not 1;

Z′ is a bond, C₁-C₆ alkylene, C₂-C₆ alkenylene, —C(═O)—, —CH(CN)— or —CH₂C(═O)NR₄—;

R₁ is

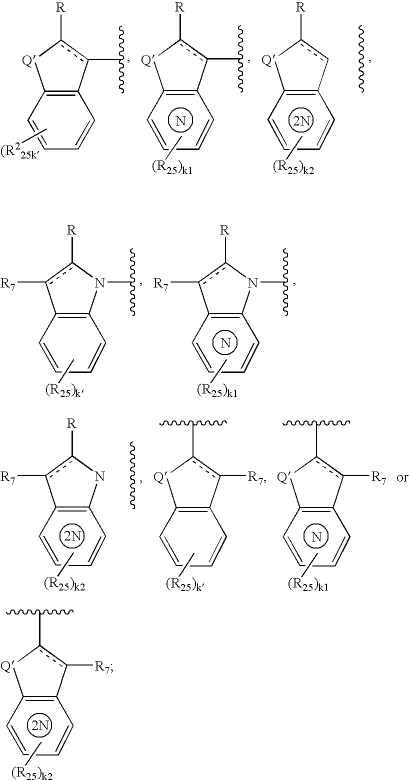

Q′ is —N(R₈)—, —S— or —O—;

k′ is 0, 1, 2, 3 or 4;

k1 is 0, 1, 2 or 3;

k2 is 0, 1 or 2;

the dotted line represents an optional double bond;

R and R₇ are independently selected from the group consisting of H, C₁-C₆ alkyl, halo(C₁-C₆)alkyl-, C₁-C₆ alkoxy, (C₁-C₆)alkoxy-(C₁-C₆)alkyl-, (C₁-C₆)-alkoxy-(C₁-C₆)alkoxy, (C₁-C₆)alkoxy-(C₁-C₆)alkyl-SO₀₋₂, R₃₂-aryl(C₁-C₆)alkoxy-, R₃₂-aryl-(C₁-C₆)alkyl-, R₃₂-aryl, R₃₂-aryloxy, R₃₂-heteroaryl, (C₃-C₆)cycloalkyl, (C₃-C₆)cycloalkyl-(C₁-C₆)alkyl, (C₃-C₆)cycloalkyl-(C₁-C₆)alkoxy, (C₃-C₆)cycloalkyl-oxy-, R₃₇-heterocyclo-alkyl, N(R₃₀)(R₃₁)—(C₁-C₆)alkyl-, —N(R₃₀)(R₃₁), —NH—(C₁-C₆)alkyl-O—(C₁-C₆)alkyl, —NHC(O)NH(R₂₉); R₂₉—S(O)₀₋₂—, halo(C₁-C₆)alkyl-S(O)₀₋₂—, N(R₃₀)(R₃₁)—(C₁-C₆)alkyl-S(O)₀₋₂—, benzoyl, (C₁-C₆)alkoxy-carbonyl, R₃₇-heterocycloalkyl-N(R₂₉)—C(O)—, (C₁-C₆)alkyl-N(R₂₉)—C(O)—, (C₁-C₆)alkyl-N(C₁-C₆ alkoxy)-C(O)— and —C(═NOR₃₆)R₃₆; and when the optional double bond is not present, R₇ can be OH;

R₈ is H, C₁-C₆ alkyl, halo(C₁-C₆)alkyl-, (C₁-C₆)alkoxy-(C₂-C₆)alkyl-, R₃₂-aryl(C₁-C₆)alkyl-, R₃₂-aryl, R₃₂-heteroaryl, R₃₂-heteroaryl(C₁-C₆)alkyl-, (C₃-C₆)cycloalkyl, (C₃-C₆)cycloalkyl-(C₁-C₆)alkyl, R₃₇-heterocycloalkyl, R₃₇-heterocycloalkyl(C₁-C₆)alkyl, N(R₃₀)(R₃₁)—(C₂-C₆)alkyl-, R₂₉—S(O)₂—, halo(C₁-C₆)alkyl-S(O)₂—, R₂₉—S(O)₀₋₁—(C₂-C₆)alkyl-, halo(C₁-C₆)alkyl-S(O)₀₋₁—(C₂-C₆)alkyl-, (C₁-C₆)alkyl-N(R₂₉)—SO₂—, or R₃₂-heteroaryl-SO₂;

R₂ is a six-membered heteroaryl ring having 1 or 2 heteroatoms independently selected from N or N—O, with the remaining ring atoms being carbon; a five-membered heteroaryl ring having 1, 2 or 3 heteroatoms independently selected from N, O or S, with the remaining ring atoms being carbon; R₃₂-quinolyl; R₃₂-aryl; 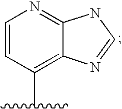
or heterocycloalkyl; wherein said six-membered heteroaryl ring or said five-membered heteroaryl ring is optionally substituted by R₆;

R₃ is H, halogen, C₁-C₆ alkyl, —OH or (C₁-C₆)alkoxy;

R₄ is independently selected from the group consisting of hydrogen, C₁-C₆ alkyl, C₃-C₆ cycloalkyl, (C₃-C₆)cycloalkyl(C₁-C₆)alkyl, R₃₃-aryl, R₃₃-aryl(C₁-C₆)alkyl, and R₃₃-heteroaryl;

R₅ is hydrogen, C₁-C₆ alkyl, —C(O)R₂₀, —C(O)₂R₂₀, —C(O)N(R₂₀)₂, R₃₃-aryl(C₁-C₆)alkyl or (C₁-C₆)alkyl-SO₂—;

R₆ is 1 to 3 substituents independently selected from the group consisting of —OH, halogen, C₁-C₆ alkyl, C₁-C₆ alkoxy, —CF₃, —NR₄R₅, —(C₁-C₆)alkyl-NR₄R₅, phenyl, R₃₃-phenyl, NO₂, —CO₂R₄, —CON(R₄)₂, —NHC(O)N(R₄)₂, R₃₂-heteroaryl-SO₂—NH—, R₃₂-aryl-(C₁-C₆)alkyl-NH—, R₃₂-heteroaryl-(C₁-C₆)alkyl-NH—, R₃₂-heteroaryl-NH—C(O)—NH— and R₃₇-heterocyclo-alkyl-N(R₂₉)—C(O)—;

R₁₂ is independently selected from the group consisting of C₁-C₆ alkyl, hydroxyl, C₁-C₆ alkoxy, or fluoro, provided that when R₁₂ is hydroxy or fluoro, then R₁₂ is not bound to a carbon adjacent to a nitrogen; or R₁₂ forms a C₁ to C₂ alkyl bridge from one ring carbon to another ring carbon;

R₁₃ is independently selected from the group consisting of C₁-C₆ alkyl, hydroxyl, C₁-C₆ alkoxy, or fluoro, provided that when R₁₃ is hydroxy or fluoro then R₁₃ is not bound to a carbon adjacent to a nitrogen; or forms a C₁ to C₂ alkyl bridge from one ring carbon to another ring carbon; or R₁₃ is ═O;

R₂₀ is independently selected from the group consisting of hydrogen, C₁-C₆ alkyl, or aryl, wherein said aryl group is optionally substituted with from 1 to 3 groups independently selected from halogen, —CF₃, —OCF₃, hydroxyl, or methoxy; or when two R₂₀ groups are present, said two R₂₀ groups taken together with the nitrogen to which they are bound can form a five or six membered heterocyclic ring;

R₂₂ is C₁-C₆ alkyl, R₃₄-aryl or heterocycloalkyl;

R₂₄ is H, C₁-C₆ alkyl, —SO₂R₂₂ or R₃₄-aryl;

R₂₅ is independently selected from the group consisting of C₁-C₆ alkyl, halogen, —CF₃, —OH, C₁-C₆ alkoxy, (C₁-C₆)alkyl-C(O), aryl-C(O)—, N(R₄)(R₅)—C(O)—, N(R₄)(R₅)—S(O)₁₋₂—, halo-(C₁-C₆)alkyl- or halo-(C₁-C₆)alkoxy-(C₁-C₆)alkyl-;

R₂₉ is H, C₁-C₆ alkyl, R₃₅-aryl or R₃₅-aryl(C₁-C₆)alkyl-;

R₃₀ is H, C₁-C₆ alkyl-, R₃₅-aryl or R₃₅-aryl(C₁-C₆)alkyl-;

R₃₁ is H, C₁-C₆ alkyl-, R₃₅-aryl, R₃₅-aryl(C₁-C₆)alkyl-, (C₁-C₆)alkyl-C(O)—, R₃₅-aryl-C(O), N(R₄)(R₅)—C(O)—, (C₁-C₆)alkyl-S(O)₂— or R₃₅-aryl-S(O)₂—;

or R₃₀ and R₃₁ together are —(CH₂)₄₋₅—, —CH₂)₂—O—(CH₂)₂— or —CH₂)₂—N(R₂₉)—(CH₂)₂— and form a ring with the nitrogen to which they are attached;

R₃₂ is 1 to 3 substituents independently selected from the group consisting of H, —OH, halogen, C₁-C₆ alkyl, C₁-C₆ alkoxy, R₃₅-aryl-O—, —SR₂₂, —CF₃, —OCF₃, —OCHF₂, —NR₄R₅, phenyl, R₃₃-phenyl, NO₂, —CO₂R₄, —CON(R₄)₂, —S(O)₂R₂₂, —S(O)₂N(R₂₀)₂, —N(R₂₄)S(O)₂R₂₂, —CN, hydroxy-(C₁-C₆)alkyl-, —OCH₂CH₂OR₂₂, and R₃₅-aryl(C₁-C₆)alkyl-O—, wherein said aryl group is optionally substituted with 1 to 3 independently selected halogens;

R₃₃ is 1 to 3 substituents independently selected from the group consisting of C₁-C₆ alkyl, halogen, —CN, —NO₂, —OCHF₂ and —O—(C₁-C₆)alkyl;

R₃₄ is 1 to 3 substituents independently selected from the group consisting of H, halogen, —CF₃, —OCF₃, —OH and —OCH₃.

R₃₅ is 1 to 3 substituents independently selected from the group consisting of hydrogen, halo, C₁-C₆ alkyl, hydroxy, C₁-C₆ alkoxy, phenoxy, —CF₃, —N(R₃₆)₂, —COOR₂₀ and —NO₂;

R₃₆ is independently selected from the group consisting of H and C₁-C₆ alkyl; and

R₃₇ is independently selected from the group consisting of H, C₁-C₆ alkyl and (C₁-C₆)alkoxycarbonyl.

Preferred compounds of formula XV include the following wherein:

R₁ is preferably 3-indolyl or 1-indolyl. The double bond is preferably present in the R₁ substituent.

R is preferably H, alkyl, R₃₂-aryl, R₃₂-heteroaryl, (C₁-C₆)alkoxy-carbonyl or (C₁-C₆)alkyl-N(R₂₉)—C(O)—. When R is (C₁-C₆)alkyl-N(R₂₉)—C(O), R₂₉ is preferably H or C₁-C₆ alkyl. More preferably, R is R₃₂-aryl or R₃₂-heteroaryl. Especially preferred are R₃₂-phenyl and R₃₂-pyridyl. R₇ is preferably H.

R₈ is preferably H, R₃₂-aryl(C₁-C₆)alkyl-, R₃₂-heteroaryl(C₁-C₆)alkyl-, R₃₂-aryl, R₃₂-heteroaryl, (C₁-C₆)alkyl-N(R₂₉)—SO₂— or R₃₂-heterocycloalkyl(C₁-C₆)alkyl-. Especially preferred are H, R₃₂-benzyl, R₃₂-pyridylmethyl, (C₁-C₆)alkyl-N(R₂₉)—SO₂— wherein R₂₉ is H or C₁-C₆ alkyl, and piperidinoethyl.

R₂₅ is preferably H, halogen or —CF₃ and k′ is 0 or 1. When R¹ is an aza- or diaza derivative of indole, R is preferably as defined above, and k1 and k2 are preferably zero.

X′ is preferably a bond.

R₂ is preferably a six-membered heteroaryl ring, optionally substituted with one substituent. More preferably, R₂ is pyridyl, pyrimidyl or pyridazinyl, optionally substituted with —NH₂.

Y′ is preferably —C(O)—.

Z′ is preferably straight or branched C₁-C₃ alkyl. Methylene is an especially preferred Z group.

M¹ is preferably N; a′ is preferably 0; and n′ is preferably 2; the optional double bond in the ring containing M¹ is preferably not present (i.e., a single bond is present).

M² is preferably C(R₃) wherein R₃ is hydrogen or fluoro; b′ is preferably 0; r′ is preferably 1; and p′ is preferably 2.

Methods for making compounds of formula XV are well known to those skilled in the art. Non-limiting examples of suitable methods are disclosed in US Publication No US 2004/0019099A1, herein incorporated by reference.

In yet another embodiment, this invention provides for compositions, therapeutic combinations and methods wherein at least one H₃ receptor antagonist/inverse agonist is a compound of formula XVI: 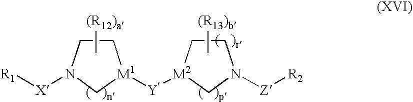
or a pharmaceutically acceptable salt or solvate thereof, wherein:

(A) R₁ is selected from:
(1) aryl;
(2) heteroaryl;
(3) heterocycloalkyl
(4) alkyl;
(5) —C(O)N(R^{4B})₂;
(6) cycloalkyl;
(7) arylalkyl;
(8) heteroarylheteroaryl (e.g., isoxazoylthienyl or pyridylthienyl); or
(9) a group selected from: 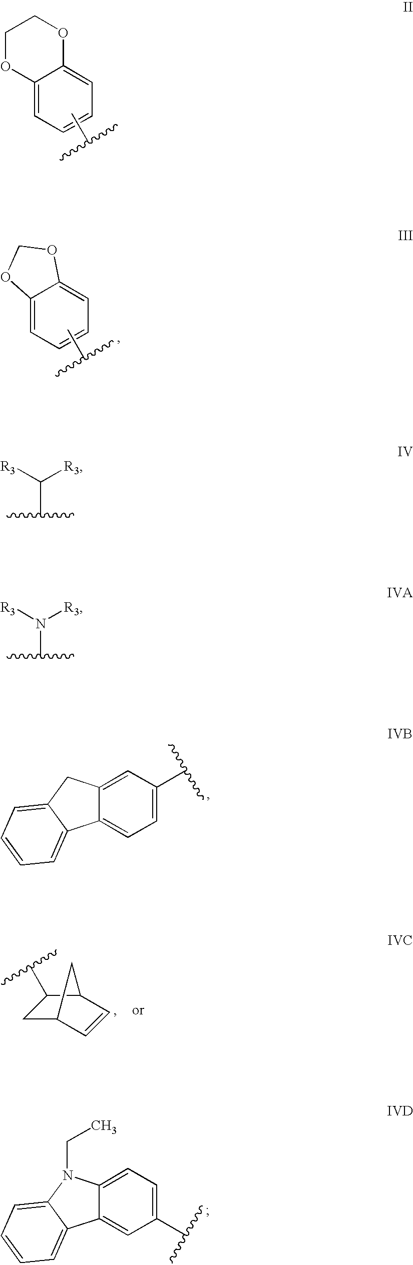
   said aryl (see (A)(1) above), heteroaryl (see (A)(2) above), aryl portion of arylalkyl (see (A)(7) above), phenyl ring of formula II (see (A)(9) above), phenyl ring of formula III (see (A)(9) above), phenyl rings of formula IVB (see (A)(9) above), or phenyl rings of formula IVD (see (A)(9) above) are optionally substituted with 1 to 3 substituents independently selected from:
(1) halogen (e.g., Br, F, or Cl, preferably F or Cl);
(2) hydroxyl (i.e., —OH);
(3) lower alkoxy (e.g., C₁ to C₆ alkoxy, preferably C₁ to C₄ alkoxy, more preferably C₁ to C₂ alkoxy, most preferably methoxy);
(4) —Oaryl (i.e., aryloxy);
(5) —SR₂₂;
(6) —CF₃;
(7) —OCF₃;
(8) —OCHF₂;
(9) —NR₄R₅;
(10) phenyl;
(11) NO₂,
(12) —CO₂R₄;
(13) —CON(R₄)₂ wherein each R₄ is the same or different;
(14) —S(O)₂R₂₂;
(15) —S(O)₂N(R₂₀)₂ wherein each R₂₀ is the same or different;
(16) —N(R₂₄)S(O)₂R₂₂;
(17) —CN;
(18) —CH₂OH;
(19) —OCH₂CH₂OR₂₂;
(20) alkyl (e.g., C₁ to C₄, such as methyl);
(21) substituted phenyl wherein said phenyl has 1 to 3 substituents independently selected from alkyl, halogen, —CN, —NO₂, —OCHF₂, —Oalkyl;
(22) —Oalkylaryl (preferably —Oalkylphenyl or —Oalkyl-substituted phenyl, e.g., —OCH₂dichlorophenyl, such as —OCH₂-2,6-dichlorophenyl or —OCH₂-2-chloro-6-fluorophenyl) wherein said aryl group is optionally substituted with 1 to 3 independently selected halogens; or
(23) phenyl;

(B) X′ is selected from alkyl (e.g., —(CH₂)_{q′}— or branched alkyl) or —S(O)₂—;

(C) Y′ represents
(1) a single bond (i.e., Y′ represents a direct bond from M¹ to M²); or
(2) Y′ is selected from —C(O)—, —C(S)—, —(CH₂)_{q′}—, or —NR₄C(O)—; with the provisos that:
   (a) when M¹ is N, then Y′ is not —NR₄C(O)—; and
   (b) when Y′ is a bond, then M¹ and M² are both carbon;

(D) M¹ and M² are independently selected from C or N;

(E) Z′ is selected from: C₁-C₆ alkyl, —SO₂—, —C(O)— or —C(O)NR₄—;

(F) R₂ is selected from:
(1) a six-membered heteroaryl ring having 1 or 2 heteroatoms independently selected from N or N—O (i.e., N-oxide), with the remaining ring atoms being carbon;
(2) a five-membered heteroaryl ring having 1 to 3 heteroatoms selected from nitrogen, oxygen, or sulfur with the remaining ring atoms being carbon; or
(3) an alkyl group, preferably a C₁ to C₄ alkyl group, more preferably methyl;
(4) an aryl group, e.g., phenyl or substituted phenyl (preferably phenyl), wherein said substituted phenyl is substituted with 1 to 3 substituents independently selected from: halogen, —Oalkyl, —OCF₃, —CF₃, —CN, —NO₂, —NHC(O)CH₃, or —O(CH₂)_{q′}N(R^{10A})₂;
(5) —N(R_{11A})₂ wherein each R^{11A} is independently selected from: H, alkyl (e.g., i-propyl) or aryl (e.g., phenyl), preferably one R^{11A} is H and the other is phenyl or alkyl (e.g., i-propyl);
(6) a group of the formula: 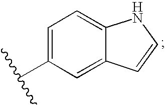
or
(7) a heteroarylheteroaryl group, e.g., 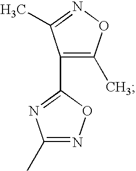
said five membered heteroaryl ring ((F)(2) above) or six-membered heteroaryl ring ((F)(1) above) is optionally substituted with 1 to 3 substituents selected from:
   (a) halogen;
   (b) hydroxyl;
   (c) lower alkyl;
   (d) lower alkoxy;
   (e) —CF₃;
   (f) —NR₄R₅;
   (g) phenyl;
   (h) —NO₂;
   (i) —C(O)N(R₄)₂ (wherein each R₄ is the same or different);
   (j) —C(O)₂R₄; or
   (k) phenyl substituted with 1 to 3 substituents independently selected from: halogen, —Oalkyl, —OCF₃, —CF₃, —CN, —NO₂ or —O(CH₂)_{q}N(R^{10A})₂;

(G) R₃ is selected from:
(1) aryl;
(2) heteroaryl;
(3) heterocycloalkyl
(4) alkyl; or
(5) cycloalkyl;
wherein said aryl or heteroaryl R₃ groups is optionally substituted with 1 to 3 substituents independently selected from:
   (a) halogen (e.g., Br, F, or Cl, preferably F or Cl);
   (b) hydroxyl (i.e., —OH);
   (c) lower alkoxy (e.g., C₁ to C₆ alkoxy, preferably C₁ to C₄ alkoxy, more preferably C₁ to C₂ alkoxy, most preferably methoxy);
   (d) —Oaryl (i.e., aryloxy);
   (e) —SR₂₂;
   (f) —CF₃;
   (g) —OCF₃;
   (h) —OCHF₂;
   (i) —NR₄R₅;
   (j) phenyl;
   (k) —NO₂,
   (l) —CO₂R₄;
   (m) —CON(R₄)₂ wherein each R₄ is the same or different;
   (n) —S(O)₂R₂₂;
   (o) —S(O)₂N(R₂₀)₂ wherein each R₂₀ is the same or different;
   (p) —N(R₂₄)S(O)₂R₂₂;
   (q) —CN;
   (r) —CH₂OH;
   (s) —OCH₂CH₂OR₂₂; or
   (t) alkyl;

(H) R₄ is selected from:
(1) hydrogen;
(2) C₁-C₆ alkyl;
(3) cycloalkyl;
(4) cycloalkylalkyl (e.g., cyclopropyl-CH₂— or cyclohexyl-CH₂—);
(5) heterocycloalkylalky (e.g., tetrahydrofuranyl-CH₂—);
(6) bridged bicyclic cycloalkyl ring, such as, for example: 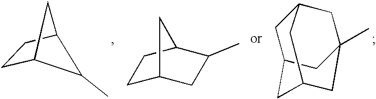
(7) aryl having a fused heterocycloalkyl ring bound to said aryl ring, preferably the heteroatoms in said heterocycloalkyl ring are two oxygen atoms, e.g., phenyl having a heterocycloalkyl ring bound to said phenyl ring, such as 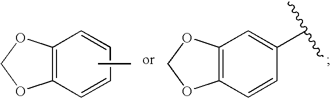
(8) aryl;
(9) arylalkyl;
(10) alkylaryl;
(11) —(CH₂)_{d′}CH(R^{12A})₂ wherein d is 1 to 3 (preferably 1), and each R^{12A} is independently selected from phenyl or substituted phenyl, said substituted phenyl being substituted with 1 to 3 substituents independently selected from: halogen, —Oalkyl, —OCF₃, —CF₃, —CN, or —NO₂, e.g., 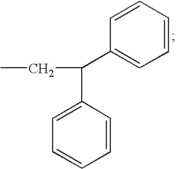
(12) heterocycloalkylheteroaryl, e.g., 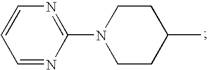
or
(13) —(C₁ to C₆)alkylene-O—R²² (e.g., —C₃H₆OCH₃);
wherein the aryl R₄ group, the aryl portion of the arylalkyl R₄ group, or the aryl portion of the alkylaryl R₄ group is optionally substituted with 1 to 3 substituents independently selected from:
   (a) halogen;
   (b) hydroxyl;
   (c) lower alkyl;
   (d) lower alkoxy;
   (e) —CF₃;
   (f) —N(R₂₀)(R₂₄),
   (g) phenyl;
   (h) —NO₂;
   (i) —C(O)N(R₂₀)₂ (wherein each R₂₀ is the same or different),
   (j) —C(O)R₂₂;
   (i) —(CH₂)_{k′}-cycloalkyl;
   (j) —(CH₂)_{q′}-aryl; or
   (k) —(CH₂)_{m′}—OR₂₂;

(I) each R^{4B} is independently selected from: H, heteroaryl (e.g., pyridyl), alkyl, alkenyl (e.g., allyl), a group of the formula 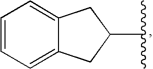
arylalkyl (e.g., benzyl), or arylalkyl wherein the aryl moiety is substituted with 1-3 substituents independently selected from: halogen (e.g. —CH₂-p-Cl-phenyl); preferably one R^{4B} is H;

(J) R₅ is selected from: hydrogen, C₁-C₆ alkyl, —C(O)₂₀ (e.g., —C(O)alkyl, such as —C(O)CH₃), —C(O)₂R₂₀, —C(O)N(R₂₀)₂ (wherein each R₂₀ is the same or different);

(K) each R^{10A} is independently selected from H or C₁ to C₆ alkyl (e.g., methyl), or each R^{10A}, taken together with the nitrogen atom to which they are bound, forms a 4 to 7 membered heterocycloalkyl ring;

(L) R₁₂ is
(1) selected from alkyl, hydroxyl, alkoxy, or fluoro, provided that when R₁₂ is hydroxy or fluoro then R₁₂ is not bound to a carbon adjacent to a nitrogen; or
(2) R₁₂ forms an alkyl bridge from one ring carbon to another ring carbon, an example of such a bridged ring system is:

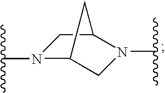

(M) R₁₃ is
(1) selected from alkyl, hydroxyl, alkoxy, or fluoro, provided that when R₁₃ is hydroxy or fluoro then R₁₃ is not bound to a carbon adjacent to a nitrogen; or
(2) R₁₃ forms an alkyl bridge from one ring carbon to another ring carbon, an example of such a bridged ring system is:

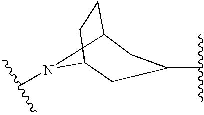

(N) R₂₀ is selected from hydrogen, alkyl, or aryl, wherein said aryl group is optionally substituted with from 1 to 3 groups independently selected from: halogen, —CF₃, —OCF₃, hydroxyl, or methoxy; or when two R₂₀ groups are present, said two R₂₀ groups taken together with the nitrogen to which they are bound form a five or six membered heterocyclic ring;

(O) R₂₂ is selected from: heterocycloalkyl (e.g., morpholinyl or pyrrolidinyl), alkyl or aryl, wherein said aryl group is optionally substituted with 1 to 3 groups independently selected from halogen, —CF₃, —OCF₃, hydroxyl, or methoxy;

(P) R₂₄ is selected from: hydrogen, alkyl, —SO₂R₂₂, or aryl, wherein said aryl group is optionally substituted with 1 to 3 groups independently selected from halogen, —CF₃, —OCF₃, hydroxyl, or methoxy;

(Q) a′ is 0 to 2;

(R) b′ is 0 to 2;

(S) k′ is 1 to 5;

(T) m′ is 2 to 5;

(U) n′ is 1, 2 or 3 with the proviso that when M¹ is N, then n′ is not 1;

(V) p′ is 1, 2 or 3 with the proviso that when M² is N, then p′ is not 1;

(W) q′ is 1 to 5; and

(X) r′ is 1, 2, or 3 with the proviso that when r′ is 2 or 3, then M² is C and p′ is 1.

Methods for making compounds of Formula XVI are well known to those skilled in the art. Non-limiting examples of suitable methods are disclosed in U.S. Pat. No. 6,849,621 B2, herein incorporated by reference.

In yet another embodiment, this invention provides for compositions, therapeutic combinations and methods of the present invention wherein at least one at least one H₃ receptor antagonist/inverse agonist is a compound of formula XVII: 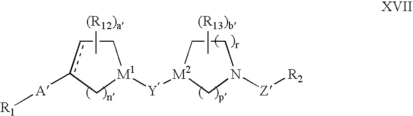
or a pharmaceutically acceptable salt or solvate thereof, wherein:

the dotted line represents an optional double bond;

a′ is 0 to 3;

b′ is 0 to 3;

n′ is 1, 2 or 3;

p′ is 1, 2 or 3;

r′ is 0, 1, 2, or 3;

with the provisos that when M² is N, p′ is not 1; and that when r′ is 0, M² is C; and that the sum of p′ and r′ is 1 to 4;

A′ is a bond or C₁-C₆ alkylene;

M¹ is CH or N;

M² is C(R₃) or N;

Y′ is —C(═O)—, —C(═S)—, —(CH₂)_{q′}—, —NR₄C(═O)—, —C(═O)NR₄—, —C(═O)CH₂—, —SO₁₋₂—, —NH—C(═N—CN)— or —C(═N—CN)—NH—; with the provisos that when M¹ is N, Y′ is not —NR₄C(═O)— or —NH—C(═N—CN)—; and when M² is N, Y′ is not —C(═O)NR₄— or —C(═N—CN)—NH—;

q′ is 1 to 5, provided that when M¹ and M² are both N, q′ is not 1;

Z′ is a bond, C₁-C₆ alkylene, C₁-C₆ alkenylene, —C(═O)—, —CH(CN)—, or —CH₂C(═O)NR₄—;

R₁ is

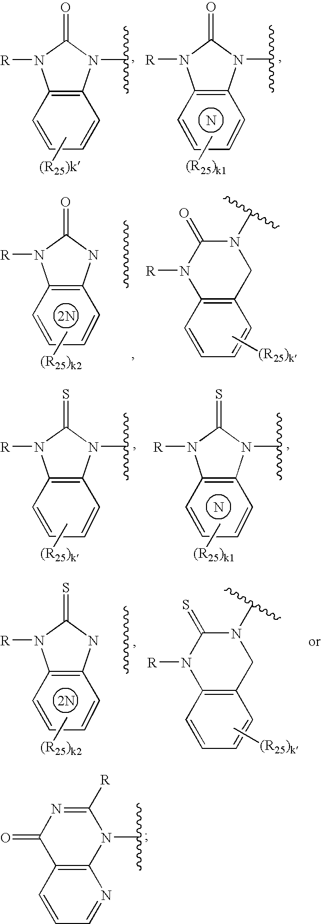

k′ is 0, 1, 2, 3 or 4;

k1 is 0, 1, 2 or 3;

k2 is 0, 1 or 2;

R is H, C₁-C₆ alkyl, hydroxy-(C₂-C₆)alkyl-, halo-(C₁-C₆)alkyl-, halo-(C₁-C₆)alkoxy-(C₁-C₆)alkyl-, R₂₉—O—C(O)—(C₁-C₆)alkyl-, (C₁-C₆)alkoxy-(C₁-C₆)alkyl-, N(R₃₀)(R₃₁)—(C₁-C₆)alkyl-, (C₁-C₆)alkoxy-(C₁-C₆)alkoxy-(C₁-C₆)alkyl-, R₃₂-aryl, R₃₂-aryl(C₁-C₆)alkyl-, R₃₂-aryloxy(C₁-C₆)alkyl-, R₃₂-heteroaryl, R₃₂-heteroaryl(C₁-C₆)alkyl-, (C₃-C₆)cycloalkyl, (C₃-C₆)cycloalkyl(C₁-C₆)alkyl-, N(R₃₀)(R₃₁)—C(O)—(C₁-C₆)alkyl-, or heterocycloalkyl(C₁-C₆)alkyl-;

R₂ is a six-membered heteroaryl ring having 1 or 2 heteroatoms independently selected from N or N—O, with the remaining ring atoms being carbon; a five-membered heteroaryl ring having 1, 2 or 3 heteroatoms independently selected from N, O or S, with the remaining ring atoms being carbon; R₃₂-quinolyl; R₃₂-aryl; heterocycloalkyl; 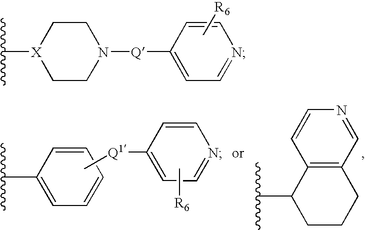
wherein said six-membered heteroaryl ring or said five-membered heteroaryl ring is optionally substituted by R₆;

X′ is C or N;

Q′ is a bond or C₁-C₆ alkylene;

Q^{1′} is a bond, C₁-C₆ alkylene or —N(R₄)—;

R₃ is H, halogen, C₁-C₆ alkyl, —OH or (C₁-C₆)alkoxy;

R₄ is independently selected from the group consisting of hydrogen, C₁-C₆ alkyl, C₃-C₆ cycloalkyl, (C₃-C₆)cycloalkyl(C₁-C₆)alkyl, R₃₃-aryl, R₃₃-aryl(C₁-C₆)alkyl, and R₃₂-heteroaryl;

R₅ is hydrogen, C₁-C₆ alkyl, —C(O)R₂₀, —C(O)₂R₂₀, —C(O)N(R₂₀)₂ or (C₁-C₆)alkyl-SO₂—;

R₆ is 1 to 3 substituents independently selected from the group consisting of —OH, halogen, C₁-C₆ alkyl-, C₁-C₆ alkoxy, C₁-C₆ alkylthio, —CF₃, —NR₄R₅, phenyl, R₃₃-phenyl, NO₂, —CO₂R₄, —CON(R₄)₂,

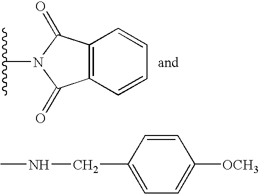

R₁₂ is independently selected from the group consisting of C₁-C₆ alkyl, hydroxyl, C₁-C₆ alkoxy, or fluoro, provided that when R₁₂ is hydroxy or fluoro, then R₁₂ is not bound to a carbon adjacent to a nitrogen; or R₁₂ forms a C₁ to C₂ alkyl bridge from one ring carbon to another ring carbon;

R₁₃ is independently selected from the group consisting of C₁-C₆ alkyl, hydroxyl, C₁-C₆ alkoxy, or fluoro, provided that when R₁₃ is hydroxy or fluoro then R¹³ is not bound to a carbon adjacent to a nitrogen; or forms a C₁ to C₂ alkyl bridge from one ring carbon to another ring carbon; or R₁₃ is ═O;

R₂₀ is independently selected from the group consisting of hydrogen, C₁-C₆ alkyl, or aryl, wherein said aryl group is optionally substituted with from 1 to 3 groups independently selected from halogen, —CF₃, —OCF₃, hydroxyl, or methoxy; or when two R₂₀ groups are present, said two R₂₀ groups taken together with the nitrogen to which they are bound form a five or six membered heterocyclic ring;

R₂₂ is C₁-C₆ alkyl, R₃₄-aryl or heterocycloalkyl;

R₂₄ is H, C₁-C₆ alkyl, —SO₂R₂₂ or R₃₄-aryl;

R₂₅ is independently selected from the group consisting of C₁-C₆ alkyl, halogen, —CF₃, —OH, C₁-C₆ alkoxy, (C₁-C₆)alkyl-C(O), aryl-C(O)—, N(R₄)(R₅)—C(O)—, N(R₄)(R₅)—S(O)₁₋₂—, halo-(C₁-C₆)alkyl- or halo-(C₁-C₆)alkoxy-(C₁-C₆)alkyl-;

R₂₉ is H, C₁-C₆ alkyl, R₃₅-aryl or R₃₅-aryl(C₁-C₆)alkyl-;

R₃₀ is H, C₁-C₆ alkyl-, R₃₅-aryl or R₃₅-aryl(C₁-C₆)alkyl-;

R₃₁ is H, C₁-C₆ alkyl-, R₃₅-aryl, R₃₅-aryl(C₁-C₆)alkyl-, (C₁-C₆)alkyl-C(O)—, R₃₅-aryl-C(O), N(R₄)(R₅)—C(O)—, (C₁-C₆)alkyl-S(O)₂— or R₃₅-aryl-S(O)₂—;

or R₃₀ and R₃₁ together are —(CH₂)₄₋₅—, —CH₂)₂—O—(CH₂)₂— or —CH₂)₂—N(R₂₉)—(CH₂)₂— and form a ring with the nitrogen to which they are attached;

R₃₂ is 1 to 3 substituents independently selected from the group consisting of H, —OH, halogen, C₁-C₆ alkyl, C₁-C₆ alkoxy, R₃₅-aryl-O—, —SR₂₂, —CF₃, —OCF₃, —OCHF₂, —NR₄R₅, phenyl, R₃₃-phenyl, NO₂, —CO₂R₄, —CON(R₄)₂, —S(O)₂R₂₂, —S(O)₂N(R₂₀)₂, —N(R₂₄)S(O)₂R₂₂, —CN, hydroxy-(C₁-C₆)alkyl-, —OCH₂CH₂OR₂₂, and R₃₅-aryl(C₁-C₆)alkyl-O—, wherein said aryl group is optionally substituted with 1 to 3 independently selected halogens;

R₃₃ is 1 to 3 substituents independently selected from the group consisting of C₁-C₆ alkyl, halogen, —CN, —NO₂, —OCHF₂ and —O—(C₁-C₆)alkyl;

R₃₄ is 1 to 3 substituents independently selected from the group consisting of H, halogen, —CF₃, —OCF₃, —OH and —OCH₃.

R₃₅ is 1 to 3 substituents independently selected from hydrogen, halo, C₁-C₆ alkyl, hydroxy, C₁-C₆ alkoxy, phenoxy, —CF₃, —N(R₃₆)₂, —COOR₂₀ and —NO₂; and

R₃₆ is independently selected form the group consisting of H and C₁-C₆ alkyl.

The more preferred compound of formula XVII include the following compounds:

R¹ is preferably R-substituted benzimidazolone, wherein R is preferably H, alkyl, alkoxyalkyl, R₃₂-aryl, R₃₂-heteroaryl or heterocycloalkylalkyl. More preferably, R is —CH₃, phenyl, 4-fluorophenyl, CH₃—O—(CH₂)₂—, 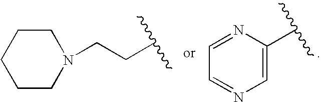
R₂₅ is preferably halogen or —CF₃ and k is 0 or 1. When R₁ is an aza- or diaza derivative of benzimidazolone, R is preferably as defined for benzimidazolone, and k₁ and k₂ are preferably zero.

R₂ is preferably a six-membered heteroaryl ring, optionally substituted with one substituent. More preferably, R₂ is pyridyl, pyrimidinyl or pyridazinyl, each optionally substituted with halogen or —NR₄R₅, wherein R₄ and R₅ are independently selected from the group consisting of H and (C₁-C₆)alkyl, or R₄ and R₅ together with the nitrogen to which they are attached form a pyrrolidinyl, piperidinyl or morpholinyl ring.

A′ is preferably a bond.

Y′ is preferably —C(O)—.

Z′ is preferably straight or branched C₁-C₃ alkyl.

M¹ is preferably N; a′ is preferably 0; and n′ is preferably 2; the optional double bond is preferably not present (i.e., a single bond is present).

M² is preferably C(R₃) wherein R₃ is hydrogen or halogen, especially fluorine; b′ is preferably 0; r′ is preferably 1; and p′ is preferably 2.

Methods for making compounds of formula XVII are well known to those skilled in the art. Non-limiting examples of suitable methods are disclosed in US Publication No US 2004/0097483A1, herein incorporated by reference.

Other non-limiting H₃ receptor antagonists/inverse agonists are disclosed in U.S. Provisional Application Ser. Nos. 60/692,110 and 60/692,175, both filed on Jun. 20, 2005, U.S. 2002/183309, 2002/177589, 2002/111340, 2004/0122033, 2003/0186963, 2003/0130253, 2004/0248938, 2002/0058659, 2003/0135056, 2003/134835, 2003/153548, 2004/0019099, 2004/0097483, 2004/0048843, 2004/087573, 2004/092521, 2004/214856, 2004/248899, 2004/224953, 2004/224952, 2005/222151, 2005/222129, 2005/182045, 2005/171181, U.S. Pat. Nos. 6,620,839, 6,515,013, 6,559,140, 6,316,475, 6,166,060, 6,448,282, 6,008,240, 5,652,258, 6,417,218, 6,673,829, 6,756,384, 6,437,147, 6,720,328, 5,869,479, 6,849,621, 6,908,929, 6,908,926, 6,906,060, 6,884,809, 6,884,803, 6,878,736, 6,638,967, 6,610,721, 6,528,522, 6,518,287, 6,506,756, 6,489,337, 6,436,939, 6,448,282, 6,407,132, 6,355,665, 6,248,765, 6,133,291, 6,103,735, 6,080,871, 5,932,596, 5,929,089, 5,837,718, 5,821,259, 5,807,872, 5,639,775, 5,708,171, 5,578,616, 5,990,147, 6,906,081, WO 95/14007, WO 99/24405 (each of which is herein incorporated by reference). Other non-limiting examples of H₃ receptor antagonists/inverse agonists are disclosed in U.S. Provisional Application Ser. No. 60/752,636 (Attorney Docket No. CV06410L01US, entitled “Phenoxypiperidines and Analogues Thereof Useful as Histamine H₃ Antagonists”, and U.S. Provisional Ser. No. 60/752637 (Attorney Docket No. CV06411L01US), entitled “Substituted Aniline Derivatives Useful as Histamine H₃ Antagonists”, both filed on the same date as this application.

The compositions, therapeutic combinations or methods of the present invention can further comprise one or more obesity control medications. Useful obesity control medications include, but are not limited to, drugs that reduce energy intake or suppress appetite, drugs that increase energy expenditure and nutrient-partitioning agents. Suitable obesity control medications include, but are not limited to, noradrenergic agents (such as diethylpropion, mazindol, phenylpropanolamine, phentermine, phendimetrazine, phendamine tartrate, methamphetamine, phendimetrazine and tartrate); CB1 receptor antagonists (such as rimonabant); topiramate; serotonergic agents (such as sibutramine, fenfluramine, dexfenfluramine, fluoxetine, fluvoxamine and paroxtine); thermogenic agents (such as ephedrine, caffeine, theophylline, and selective β3-adrenergic agonists); an alpha-blocking agent; a kainite or AMPA receptor antagonist; a leptin-lipolysis stimulated receptor; a phosphodiesterase enzyme inhibitor; a compound having nucleotide sequences of the mahogany gene; a fibroblast growth factor-10 polypeptide; a monoamine oxidase inhibitor (such as befloxatone, moclobemide, brofaromine, phenoxathine, esuprone, befol, toloxatone, pirlindol, amiflamine, sercloremine, bazinaprine, lazabemide, milacemide and caroxazone); a compound for increasing lipid metabolism (such as evodiamine compounds); and a lipase inhibitor (such as orlistat). Preferred therapeutic combinations that may be used in the methods according to the present invention include combinations comprising at least one cholesterol lowering agent, such as a stemol or 5-α-stanol according to formulae I-IV and/or an HMG-CoA reductase inhibitor, and at least one H₃ receptor antagonist/inverse agonist, such as those according to formulae XIII to XVII. Especially preferred combinations include ezetimibe and/or simvastatin as the cholesterol lowering agents, a compound of formula XIIIA-XIIIC, and orlistat.

Generally, a total dosage of the above-described obesity control medications can range from 1 to 3,000 mg/day, desirably from about 1 to 1,000 mg/day and more desirably from about 1 to 200 mg/day in single or 2-4 divided doses.

Another embodiment of the present invention is therapeutic combinations comprising two cholesterol lowering agents and an H₃ receptor antagonist/inverse agonist. Preferred combinations include cholesterol absorption inhibitors, such as those described in formulae I to XII, and an HMG-CoA reductase inhibitor, PPAR activators, nicotinic acid (niacin) and/or nicotinic acid receptor agonists, or a bile acid sequestrant. Preferred HMG-CoA reductase inhibitors include lovastatin, pravastatin, fluvastatin, simvastatin atorvastatin, cerivastatin, CI-981, pitavastatin and rosuvastatin. Other preferred cholesterol lowering agents to be used with a cholesterol absorption inhibitor, such as those described in formulae I-XII, include cholestryamine, cholestipol, clofibrate, gemfibrozil, and fenofibrate. Preferred H₃ receptor antagonists/inverse agonists to be included in the therapeutic combinations include those described in formulae XIII-XVII, with the compounds of formulae XIIIA-XIIIC being especially preferred.

Especially preferred therapeutic combination is VYTORIN, which is a combination of ezetimibe and simvastatin (see U.S. Pat. No. 5,846,946, herein incorporated by reference), together with a compound of formulae XIIIA, XVIIIB or XIIIC.

Another embodiment of the present invention contemplates kits and method of treatment as described above which comprise: (a) at least one cholesterol lowering agent, such as a sterol or 5-α-stanol absorption inhibitor; and (b) at least on H₃ receptor antagonist/inverse agonists. Suitable cholesterol lowering agents include any of the compounds discussed above in formulae I-XII and suitable H₃ receptor antagonists/inverse agonists include any of the compounds discussed above in formulae XIII-XVII. A kit is contemplated when two separate units are combined: a pharmaceutical composition comprising at least one cholesterol absorption inhibitor and a separate pharmaceutical composition comprising at least one H₃ receptor antagonist/inverse agonist. The kit will preferably include directions for the administration of the separate components.

The kit form is particularly advantageous when the separate components must be administered in different dosage forms (e.g., oral and parenteral) or are administered at different dosage intervals.

Another embodiment of the present invention is the treatment, prevention or amelioration of the symptoms or the development of metabolic syndrome in a mammal in need thereof comprising the step of administering an effective amount of a therapeutic composition comprising of at least one cholesterol lowering agent and optionally at least one H₃ receptor antagonist/inverse agonist to said mammal. Metabolic syndrome is a clustering of atherosclerotic CHD risk factors including obesity, decreased HDL-C, and increased fasting plasma glucose levels, triglyceride levels and blood pressure. More preferably, the therapeutic combination comprises two or three different classes of cholesterol lowering agents, such as an azetinone (e.g. ezetimibe) an activator or agonists of PPAR (e.g., a fibrate, such as fenofibrate), or an HMG-CoA reductase inhibitor (e.g. simvastatin or atorvastatin).

Prodrugs and solvates of the compounds of the invention are also contemplated herein. The term “prodrug”, as employed herein, denotes a compound that is a drug precursor which, upon administration to a subject, undergoes chemical conversion by metabolic or chemical processes to yield a compound of formula I or a salt and/or solvate thereof. A discussion of prodrugs is provided in T. Higuchi and V. Stella, Pro-drugs as Novel Delivery Systems (1987) Volume 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

For example, if a compound of formulae I-XVII or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a compound of formulae I-XVII contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of formulae I-XVII incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)O)¹ wherein Y¹ is H, (C₁-C₆)alkyl or benzyl, —C(OY²)Y³ wherein Y² is (C₁-C₄)alkyl and Y³ is (C₁-C₆)alkyl, carboxy(C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

The compounds of formulae I-XVII may exists in unsolvated as well as solvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

“Effective amount” or “therapeutically effective amount” is meant to describe an amount of compound or a composition of the present invention effective in treating the disease state being treated and thus producing the desired therapeutic effect in a suitable patient.

The compounds of formulae I-XVII form salts which are also within the scope of this invention. Reference to a compound of formulae I-XVII herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of formulae I-XVII contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the formulae I-XVII may be formed, for example, by reacting a compound of formulae I-XVII with an amount of acid or base, such as an equivalent amount, in a medium such as one in which the salt precipitates or in an aqueous medium followed by lyophilization. Acids (and bases) which are generally considered suitable for the formation of pharmaceutically useful salts from basic (or acidic) pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; in The Orange Book (Food & Drug Administration, Washington, D.C. on their website); and P. Heinrich Stahl, Camille G. Wermuth (Eds.), Handbook of Pharmaceutical Salts: Properties, Selection, and Use, (2002) Int'l. Union of Pure and Applied Chemistry, pp. 330-331. These disclosures are incorporated herein by reference thereto.

Exemplary acid addition salts include acetates, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, methyl sulfates, 2-naphthalenesulfonates, nicotinates, nitrates, oxalates, pamoates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates,) undecanoates, and the like.

Exemplary basic salts include ammonium salts, alkali metal salts such as sodium, lithium, and potassium salts, alkaline earth metal salts such as calcium and magnesium salts, aluminum salts, zinc salts, salts with organic bases (for example, organic amines) such as benzathines, diethylamine, dicyclohexylamines, hydrabamines (formed with N,N-bis(dehydroabietyl)ethylenediamine), N-methyl-D-glucamines, N-methyl-D-glucamides, t-butyl amines, piperazine, phenylcyclohexylamine, choline, tromethamine, and salts with amino acids such as arginine, lysine and the like. Basic nitrogen-containing groups may be quarternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

All stereoisomers (for example, geometric isomers, optical isomers and the like) of the present compounds (including those of the salts, solvates and prodrugs of the compounds as well as the salts and solvates of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. For example, if a compound formulae I-XVII incorporates a double bond or a fused ring, both the cis- and trans-forms, as well as mixtures, are embraced within the scope of the invention. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate” “prodrug” and the like, is intended to equally apply to the salt, solvate and prodrug of enantiomers, stereoisomers, rotamers, tautomers, racemates or prodrugs of the inventive compounds.

Diasteromeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diasteromeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of formulae I-XVII may be atropisomers (e.g., substituted biaryls) and are considered as part of this invention. Enantiomers can also be separated by use of chiral HPLC column.

Polymorphic forms of the compounds of formulae I-XVII, and of the salts, solvates and prodrugs of the compounds of formulae -XVII, are intended to be included in the present invention

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of formulae I-XVII (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formulae I-XVII can generally be prepared by following procedures analogous to those disclosed in the art, by substituting an appropriate isotopically labelled reagent for a non-isotopically labelled reagent.

It should be noted that throughout the specification and Claims appended hereto any formula, compound, moiety or chemical illustration with unsatisfied valences is assumed to have the hydrogen atom to satisfy the valences unless the context indicates a bond.

The term “therapeutically effective amount” means that amount of therapeutic agents of the invention, such as the substituted azetidinone(s) and H₃ receptor antagonist/inverse agonist and other pharmacological or therapeutic agents which may be present that will elicit a biological or medical response of a subject, tissue, system, animal or mammal that is being sought by the administrator (such as a researcher, doctor or veterinarian) which includes alleviation of the symptoms, prevention, slowing or halting of progression of one or more conditions associated with NAFLD.

The daily dose of the compound of formulae I-XVII administered to the mammal can range from about 1 to about 1000 mg per day, preferably about 1 to about mg/day, and more preferably about 100 mg per day, given in a single dose or 2-4 divided doses. The exact dose, however, is determined by the attending clinician and is dependent on the potency of the compound administered, the age, weight, condition and response of the patient.

For administration of pharmaceutically acceptable salts of the above compounds, the weights indicated above refer to the weight of the acid equivalent or the base equivalent of the therapeutic compound derived from the salt.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 0.1 to about 7.5 percent active ingredient. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions, suspensions and solids in powder form, which may be in combination with a pharmaceutically acceptable carrier, such as a compressed gas, e.g. HFA.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 to about 500 mg, preferably from about 1 mg to about 250 mg, more preferably from about 1 mg to about 100 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 100 mg/day, in two to four divided doses.

Some useful terms are described below:

Capsule—refers to a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

Tablet—refers to a compressed or molded solid dosage form containing the active ingredients with suitable diluents. The tablet can be prepared by compression of mixtures or granulations obtained by wet granulation, dry granulation or dry blending.

Oral gels—refers to the active ingredients dispersed or solubilized in a hydrophillic semi-solid matrix.

Powders for constitution—refers to powder blends containing the active ingredients and suitable diluents which can be suspended or solubilized in water or juices.

Diluent—refers to substances that usually make up the major portion of the composition or dosage form. Suitable diluents include sugars such as lactose, sucrose, mannitol and sorbitol; starches derived from wheat, corn, rice and potato; and celluloses such as microcrystalline cellulose. The amount of diluent in the composition can range from about 10 to about 90% by weight of the total composition, preferably from about 25 to about 75%, more preferably from about 30 to about 60% by weight, even more preferably from about 12 to about 60%.

Disintegrants—refers to materials added to the composition to help it break apart (disintegrate) and release the medicaments. Suitable disintegrants include starches; “cold water soluble” modified starches such as sodium carboxymethyl starch; natural and synthetic gums such as locust bean, karaya, guar, tragacanth and agar; cellulose derivatives such as methylcellulose and sodium carboxymethylcellulose; microcrystalline celluloses and cross-linked microcrystalline celluloses such as sodium croscarmellose; alginates such as alginic acid and sodium alginate; clays such as bentonites; and effervescent mixtures. The amount of disintegrant in the composition can range from about 2 to about 15% by weight of the composition, more preferably from about 4 to about 10% by weight.

Binders—refers to substances that bind or “glue” powders together and make them cohesive by forming granules, thus serving as the “adhesive” in the formulation. Binders add cohesive strength already available in the diluent or bulking agent. Suitable binders include sugars such as sucrose; starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropylmethylcellulose; polyvinylpyrrolidone; and inorganics such as magnesium aluminum silicate. The amount of binder in the composition can range from about 2 to about 20% by weight of the composition, more preferably from about 3 to about 10% by weight, even more preferably from about 3 to about 6% by weight.

Lubricant—refers to a substance added to the dosage form to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and d'l-leucine. Lubricants are usually added at the very last step before compression, since they must be present on the surfaces of the granules and in between them and the parts of the tablet press. The amount of lubricant in the composition can range from about 0.2 to about 5% by weight of the composition, preferably from about 0.5 to about 2%, more preferably from about 0.3 to about 1.5% by weight.

Glidents—materials that prevent caking and improve the flow characteristics of granulations, so that flow is smooth and uniform. Suitable glidents include silicon dioxide and talc. The amount of glident in the composition can range from about 0.1% to about 5% by weight of the total composition, preferably from about 0.5 to about 2% by weight.

Coloring agents—excipients that provide coloration to the composition or the dosage form. Such excipients can include food grade dyes and food grade dyes adsorbed onto a suitable adsorbent such as clay or aluminum oxide. The amount of the coloring agent can vary from about 0.1 to about 5% by weight of the composition, preferably from about 0.1 to about 1%.

Bioavailability—refers to the rate and extent to which the active drug ingredient or therapeutic moiety is absorbed into the systemic circulation from an administered dosage form as compared to a standard or control.

### EXAMPLES

The following non-limiting example illustrates the invention.

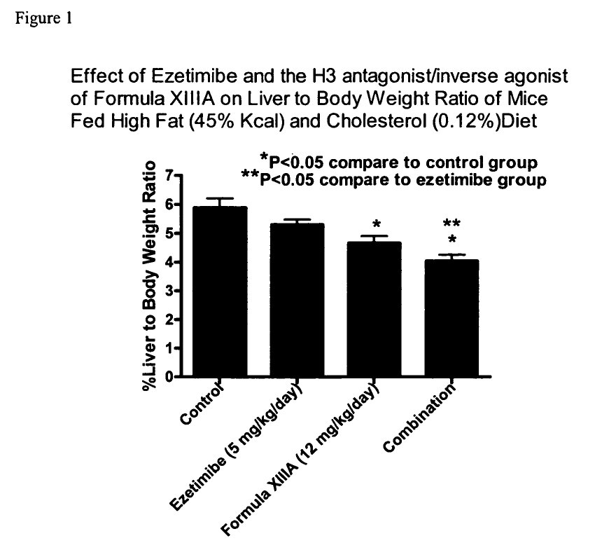

Diet-induced obese (DIO) mice, which had developed obesity, hepatic steatosis and dyslipidemia by feeding them a western diet confining 45% fat and 0.12% cholesterol for six months, were divided into four groups and treated with nothing (control), ezetimibe (formula II) (5 mg/kg/day), the compound of formula XIIIA (12 mg/kg/day), or a combination of ezetimibe (5 mgkg/day) and compound of formula XIIIA (12 mg/kg/day) for four weeks. The mice were sacrificed and the liver weight, liver triglyceride level and liver free cholesterol content were determined for each group were determined and summarized in FIGS. 1 to 4.

FIG. 1 depicts the liver to body weight ratio of each of the four groups. Mice that received ezetimibe and/or a compound of formula XIIIA showed a decrease in liver weight, with the group receiving the combination showing the greatest decline in weight.

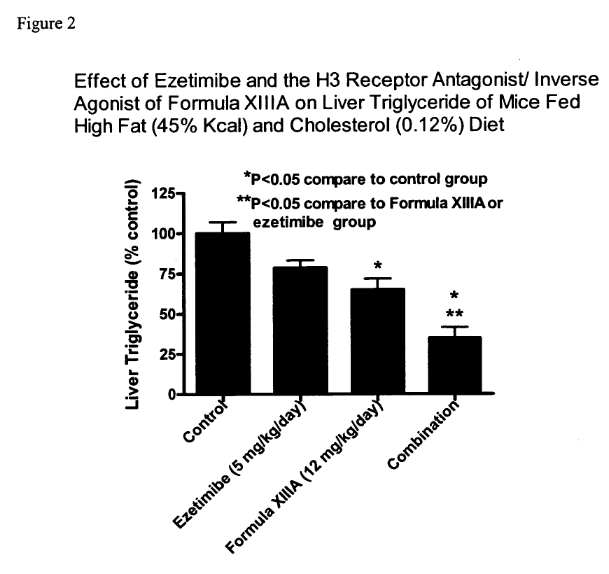

FIG. 2 depicts the triglyceride levels for each of the four groups. Mice that received ezetimibe and/or the compound of formula XIIIA all showed a decrease in liver triglyceride levels, with the group receiving the combination showing the greatest decrease.

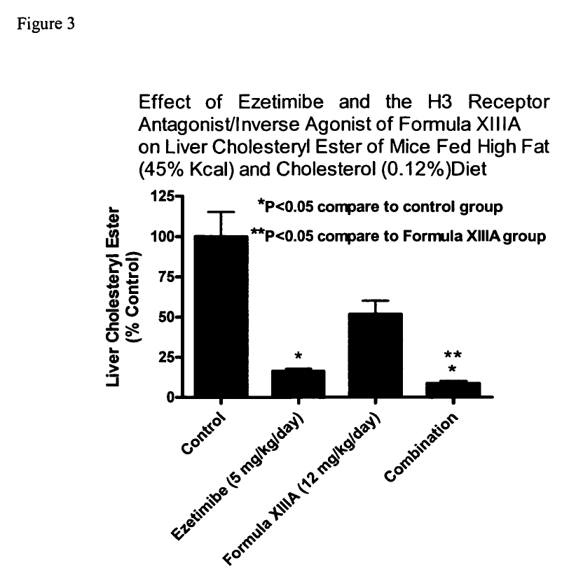

FIG. 3 depicts the liver cholesterol ester content of each of the four groups. Again, all groups that received ezetimibe and/or the compound of XIIIA showed a decrease in liver cholesteryl ester content compared to the control with the group receiving the combinations showing the greatest decrease.

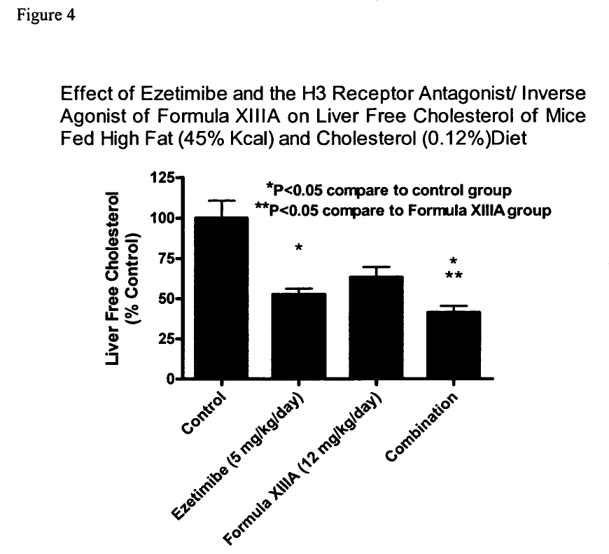

FIG. 4 depicts the liver free cholesterol content for each of the four groups. All groups that received ezetimibe and/or the compound of formula XIIIA show a decrease in liver cholesterol content compared to the control group, with the group receiving the combination showing the greatest decrease.

The data indicate that ezetimibe, the H₃ antagonist/inverse agonist of formula XIIIA and the combination of both therapeutic agents are effective in treating NAFLD, with the combination showing a synergistic effect.

### Example 2

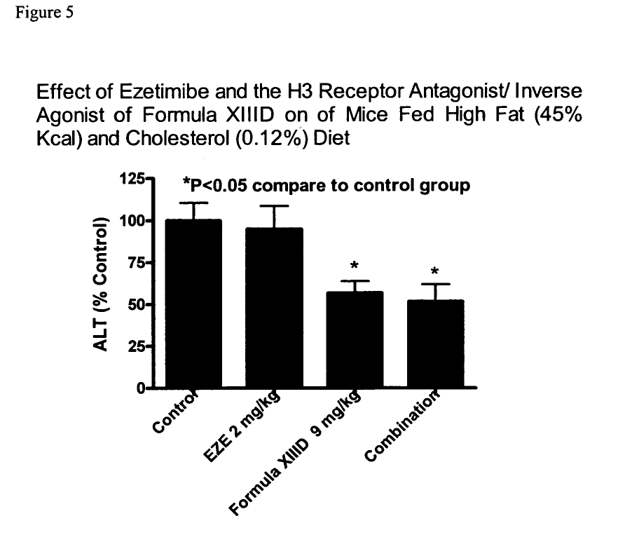

Diet induced obese (DIO) mice, which had developed obesity, dyslipidemia, hepatic steatosis and fibrosis by feeding them a western diet containing 45% fat and 0.12% cholesterol for seven months, were divided into four groups and treated with nothing (control), ezetimibe (formula II) (2 mg/kg/day), the compound of formula XIIID (9 mg/kg/day) or a combination of ezetimibe (2 mg/kg/day) and compound of formula XIIID (9 mg/kg/day) for four weeks. The mice were sacrificed and the plasma alanine aminotransferase (ALT) enzyme activities, a plasma biomarker of liver injury with steatohepatitis, were determined for each group, this is summarized in FIG. 5.

FIG. 5 depicts the plasma alanine aminotransferase (ALT) enzyme activities of each of the four groups. Mice that received a compound of formula XIIID showed a decrease in plasma ALT.

The data indicate that the H₃ antagonist/inverse agonist of formula XIIID and the combination of both the compound of formula XIIID and ezetimibe can improve liver injury biomarker ALT and, therefore, are effective in treating NASH.

Example 3

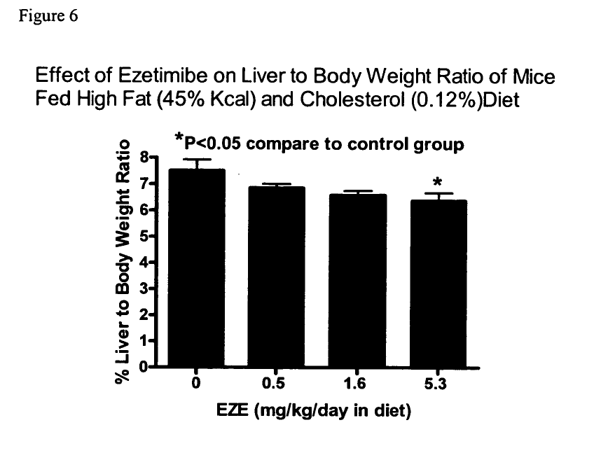

C₅₇BL/6J mice were fed a high fat/cholesterol diet (Research Diets, with 45% Kcal fat and 0.12% w/w cholesterol) for 7 months after weaning. After 4 weeks, the body weight of DIO mice treated with ezetimibe (0, 0.5, 1.6 and 5.3 mg/kb/day in the high fat/cholesterol diet) were not significantly different from control mice. However, liver wet weight and the liver to body weight ratio were significantly reduced in the ezetimibe-treated DIO mice as compared to the control. Livers from ezetimibe-treated mice had significantly lower cholesteryl esters free cholesterol and triglyceride These data are summarized in FIG. 6 to 9.

FIG. 6 depicts the liver to body weight ratio of each of the four groups. Mice that received ezetimibe showed a dose-dependent decrease in liver to body weight ratio, with the group receiving the ezetimibe (53. mg/kg/day) showing the greatest decline in liver to body weight ratio.

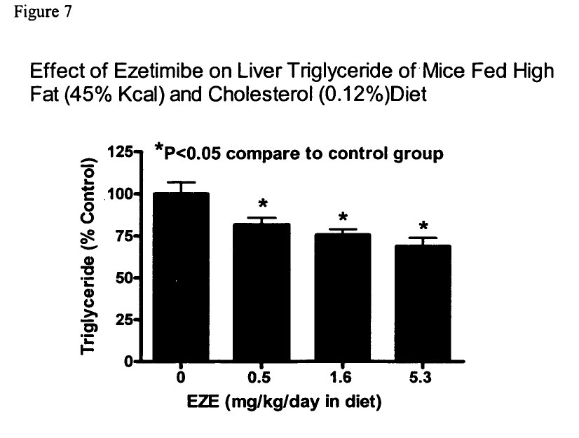

FIG. 7 depicts the triglyceride levels for each of the four groups. All groups received ezetimibe showed a dose-dependent decrease in liver triglyceride levels.

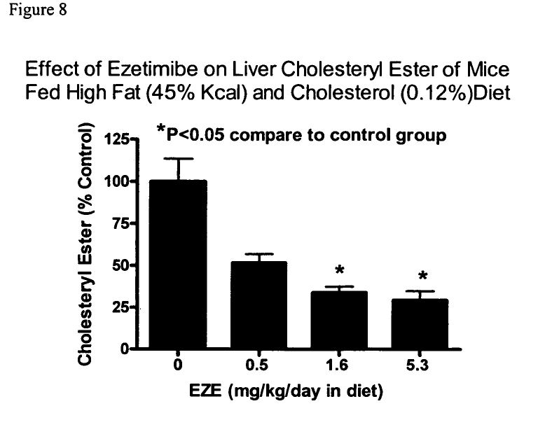

FIG. 8 depicts the liver cholesteryl ester content for each of the four groups. Mice that received ezetimibe (1.6 and 5.3 mg/kg/day) showed a significant decrease in liver cholesteryl ester content compared to the control group.

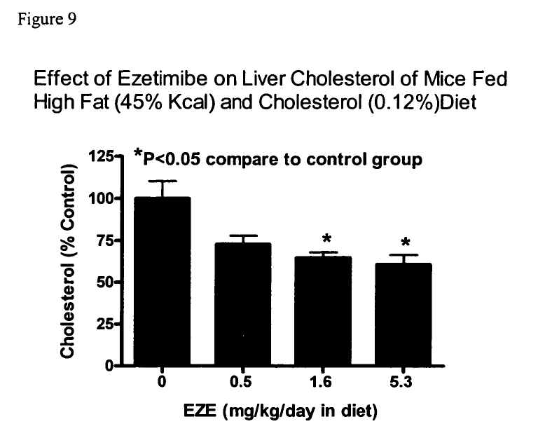

FIG. 9 depicts the liver cholesterol content of each of the four groups. Mice that received ezetimibe (1.6 and 5.3 mg/kg/day) showed a significant decrease in liver cholesterol content compared to the control group.

After 4 weeks of ezetimibe treatment, total plasma cholesterol and triglyceride was significantly reduced by 30% and 15% respectively. There were significant decreases in VLDL-C and LDL-C, while HDL-C was not changed in the ezetimibe-treated group. Although the VLDL-C was significantly reduced after ezetimibe treatment, the VLDL-TG production rates were comparable between ezetimibe-treated and control DIO mice. From this result one may conclude that ezetimibe may be used in the prevention of treatment of hepatic steatosis in a mammal.

It will be appreciated by those skilled in the art that changes could be made to the embodiments described above without departing from the broad inventive concept thereof. It is understood, therefrom that this invention is not limited to the particular embodiments disclosed, but it is intended to cover modifications that are within the spirit and scope of the invention, as defined by the appended claims.

What is claimed is:

1. A method for the treatment, prevention or ameliorating the symptoms of nonalcoholic fatty liver disease (NAFLD) in a mammal in need thereof comprising the step of administering an effective amount of a composition comprising a therapeutic combination of at least one cholesterol lowering agent and at least one H₃ antagonist/inverse agonist.

2. The method according to claim 1, wherein the cholesterol lowering agent is a sterol or 5-α-stanol absorption inhibitor.

3. The method according to claim 2, wherein the sterol or 5-α-stanol absorption inhibitor is a compound of formula (I): 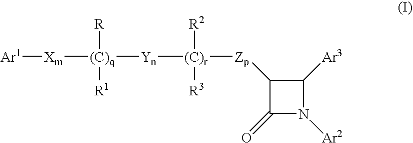
or pharmaceutically acceptable salts or solvates thereof,
wherein, in formula (I):
Ar¹ and Ar² are independently selected from the group consisting of aryl and R⁴-substituted aryl;
Ar³ is aryl or R⁵-substituted aryl;
X, Y and Z are independently selected from the group consisting of —CH₂—, —CH(lower alkyl)- and —C(dilower alkyl)-;
R and R² are independently selected from the group consisting of —OR⁶, —O(CO)R⁶, —O(CO)OR⁹ and —O(CO)NR⁶R⁷;
R¹ and R³ are independently selected from the group consisting of hydrogen, lower alkyl and aryl;
q is 0 or 1; r is 0 or 1; m, n and p are independently selected from 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5;
R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶, —CH═CH—COOR⁶, —CF₃, —CN, —NO₂ and halogen;
R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;
R⁶, R⁷ and R⁸ are independently selected from the group consisting of hydrogen, lower alkyl, aryl and aryl-substituted lower alkyl; and
R⁹ is lower alkyl, aryl or aryl-substituted lower alkyl.

4. The method according to claim 3, wherein the sterol or 5-α-stanol absorption inhibitor is a compound of formula (II): 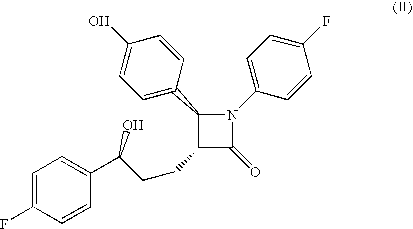
or pharmaceutically acceptable salts or solvates thereof.

5. The method according to claim 2, wherein the sterol or 5-α-stanol absorption inhibitor is a compound of formula (III): 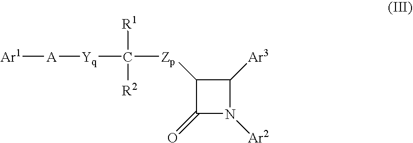
or a pharmaceutically acceptable salt thereof or a solvate thereof,
wherein, in formula (III) above:
Ar¹ is R³-substituted aryl;
Ar² is R⁴-substituted aryl;
Ar³ is R⁵-substituted aryl;
Y and Z are independently selected from the group consisting of —CH₂—, —CH(lower alkyl)- and —C(dilower alkyl)-;
A is selected from —O—, —S—, —S(O)— or —S(O)₂—,
R¹ is selected from the group consisting of —OR⁶, —O(CO)R⁶, —O(CO)OR⁹ and —O(CO)NR⁶R⁷; R² is selected from the group consisting of hydrogen, lower alkyl and aryl; or R¹ and R² together are ═O;
q is 1, 2 or 3;
p is 0, 1, 2, 3 or 4;
R⁵ is 1-3 substituents independently selected from the group consisting of —OR⁶, —O(CO)R⁶, —O(CO)OR⁶, —O(CH₂)₁₋₅OR⁹, —O(CO)NR⁶R⁷, —NR⁶R⁷, —NR⁶(CO)R⁷, —NR⁶(CO)OR⁹, —NR⁶(CO)NR⁷R⁸, —NR⁶SO₂-lower alkyl, —NR⁶SO₂-aryl, —CONR⁶R⁷, —COR⁶, —SO₂NR⁶R⁷, S(O)₀₋₂-alkyl, S(O)₀₋₂-aryl, —O(CH₂)₁₋₁₀—COOR⁶, —O(CH₂)₁₋₁₀CONR⁶R⁷, o-halogeno, m-halogeno, o-lower alkyl, m-lower alkyl, -(lower alkylene)-COOR⁶, and —CH═CH—COOR⁶;
R³ and R⁴ are independently 1-3 substituents independently selected from the group consisting of R⁵, hydrogen, p-lower alkyl, aryl, —NO₂, —CF₃ and p-halogeno;
R⁶, R⁷ and R⁸ are independently selected from the group consisting of hydrogen, lower alkyl, aryl and aryl-substituted lower alkyl; and R⁹ is lower alkyl, aryl or aryl-substituted lower alkyl.

6. The method according to claim 2, wherein the sterol or 5-α-stanol absorption inhibitor is a compound of formula (IV): 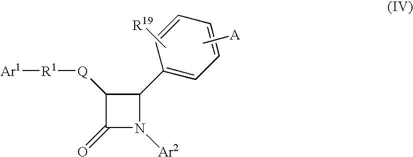
or a pharmaceutically acceptable salt thereof or a solvate thereof,
wherein, in Formula (IV):
A is selected from the group consisting of R²-substituted heterocycloalkyl, R²-substituted heteroaryl, R²-substituted benzofused heterocycloalkyl, and R²-substituted benzofused heteroaryl;
Ar¹ is aryl or R³-substituted aryl;
Ar² is aryl or R⁴-substituted aryl;
Q is a bond or, with the 3-position ring carbon of the azetidinone, forms the spiro group 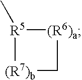
and
R¹ is selected from the group consisting of:
—(CH₂)_{q}—, wherein q is 2-6, provided that when Q forms a spiro ring, q can also be zero or 1;
—(CH₂)ₑ-G-(CH₂)ᵣ—, wherein G is —O—, —C(O)—, phenylene, —NR⁸— or —S(O)₀₋₂—, e is 0-5 and r is 0-5, provided that the sum of e and r is 1-6;
—(C₂-C₆ alkenylene)-; and
—(CH₂)_{f}—V—(CH₂)_{g}—, wherein V is C₃-C₆ cycloalkylene, f is 1-5 and g is 0-5, provided that the sum of f and g is 1-6;
R⁵ is selected from: 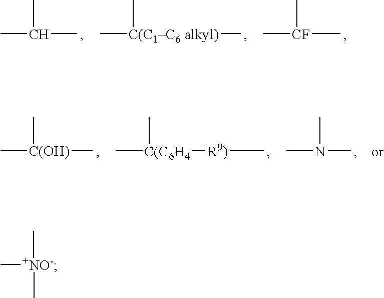
R⁶ and R⁷ are independently selected from the group consisting of —CH₂—, —CH(C₁-C₆ alkyl)-, —C(di-(C₁-C₆)alkyl), —CH═CH— and —C(C₁-C₆ alkyl)═CH—; or R⁵ together with an adjacent R⁶, or R⁵ together with an adjacent R⁷, form a —CH═CH— or a —CH═C(C₁-C₆ alkyl)- group;
a and b are independently 0, 1, 2 or 3, provided both are not zero; provided that when R⁶ is —CH═CH— or —C(C₁-C₆ alkyl)═CH—, a is 1; provided that when R⁷ is —CH═CH— or —C(C₁-C₆ alkyl)═CH—, b is 1; provided that when a is 2 or 3, the R⁶'s can be the same or different; and provided that when b is 2 or 3, the R⁷'s can be the same or different;
and when Q is a bond, R¹ also can be selected from: 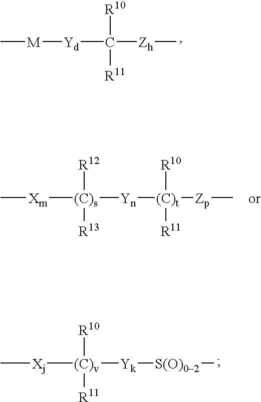
where M is —O—, —S—, —S(O)— or —S(O)₂—;
X, Y and Z are independently selected from the group consisting of —CH₂—, —CH(C₁-C₆ alkyl)- and —C(di-(C₁-C₆)alkyl);
R¹⁰ and R¹² are independently selected from the group consisting of —OR¹⁴, —O(CO)R¹⁴, —O(CO)OR¹⁶ and —O(CO)NR¹⁴R¹⁵;
R¹¹ and R¹³ are independently selected from the group consisting of hydrogen, (C₁-C₆)alkyl and aryl; or R¹⁰ and R¹¹ together are ═O, or R¹² and R¹³ together are ═O;
d is 1, 2 or 3;
h is 0, 1, 2, 3 or 4;
s is 0 or 1; t is 0 or 1; m, n and p are independently 0-4; provided that at least one of s and t is 1, and the sum of m, n, p, s and t is 1-6; provided that when p is 0 and t is 1, the sum of m, s and n is 1-5; and provided that when p is 0 and s is 1, the sum of m, t and n is 1-5;
v is 0 or 1;
j and k are independently 1-5, provided that the sum of j, k and v is 1-5;
R² is 1-3 substituents on the ring carbon atoms selected from the group consisting of hydrogen, (C₁-C₁₀)alkyl, (C₂-C₁₀)alkenyl, (C₂-C₁₀)alkynyl, (C₃-C₆)cycloalkyl, (C₃-C₆)cycloalkenyl, R¹⁷-substituted aryl, R¹⁷-substituted benzyl, R¹⁷-substituted benzyloxy, R¹⁷-substituted aryloxy, halogeno, —NR¹⁴R¹⁵, NR¹⁴R¹⁵(C₁-C₆ alkylene)—, NR¹⁴R¹⁵C(O)(C₁-C₆ alkylene)-, —NHC(O)R¹⁶, OH, C₁-C₆ alkoxy, —OC(O)R¹⁶, —COR¹⁴, hydroxy(C₁-C₆)alkyl, (C₁-C₆)alkoxy(C₁-C₆)alkyl, NO₂, —S(O)₂R¹⁶, —SO₂NR¹⁴R¹⁵ and —(C₁-C₆ alkylene)COOR¹⁴; when R² is a substituent on a heterocycloalkyl ring, R² is as defined, or is ═O or 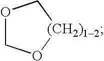
and, where R² is a substituent on a substitutable ring nitrogen, it is hydrogen, (C₁-C₆)alkyl, aryl, (C₁-C₆)alkoxy, aryloxy, (C₁-C₆)alkylcarbonyl, arylcarbonyl, hydroxy, —(CH₂)₁₋₆CONR¹⁸R¹⁸, 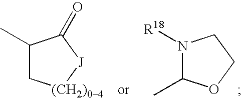
wherein J is —O—, —NH—, —NR¹⁸— or —CH₂—;
R³ and R⁴ are independently selected from the group consisting of 1-3 substituents independently selected from the group consisting of (C₁-C₆)alkyl, —OR¹⁴, —O(CO)R¹⁴, —O(CO)OR¹⁶, —O(CH₂)₁₋₅OR¹⁴, —O(CO)NR¹⁴R¹⁵, —NR¹⁴R¹⁵, —NR¹⁴(CO)R¹⁵, —NR¹⁴(CO)OR¹⁶, —NR¹⁴(CO)NR¹⁵R¹⁹, —NR¹⁴SO₂R¹⁶, —COOR¹⁴, —CONR¹⁴R¹⁵, —COR¹⁴, —SO₂NR¹⁴R¹⁵, S(O)₀₋₂R¹⁶, —O(CH₂)₁₋₁₀—COOR¹⁴, —O(CH₂)₁₋₁₀CONR¹⁴R¹⁵, —(C₁-C₆ alkylene)-COOR¹⁴, —CH═CH—COOR¹⁴, —CF₃, —CN, —NO₂ and halogen;
R⁸ is hydrogen, (C₁-C₆)alkyl, aryl(C₁-C₆)alkyl, —C(O)R¹⁴ or —COOR¹⁴;
R⁹ and R¹⁷ are independently 1-3 groups independently selected from the group consisting of hydrogen, (C₁-C₆)alkyl, (C₁-C₆)alkoxy, —COOH, NO₂, —NR¹⁴R¹⁵, OH and halogeno;
R¹⁴ and R¹⁵ are independently selected from the group consisting of hydrogen, (C₁-C₆)alkyl, aryl and aryl-substituted (C₁-C₆)alkyl;
R¹⁶ is (C₁-C₆)alkyl, aryl or R¹⁷-substituted aryl;
R¹⁸ is hydrogen or (C₁-C₆)alkyl; and
R¹⁹ is hydrogen, hydroxy or (C₁-C₆)alkoxy.

7. The method according to claim 2, wherein the sterol or 5-α-stanol absorption inhibitor is a compound of formula (V): 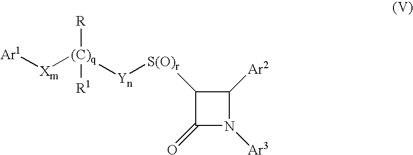
or a pharmaceutically acceptable salt thereof or a solvate thereof,
wherein, in formula (V):
Ar¹ is aryl, R¹⁰-substituted aryl, heteroaryl or R¹⁰ substituted heteroaryl;
Ar² is aryl or R⁴-substituted aryl;
Ar³ is aryl or R⁵-substituted aryl;
X and Y are independently selected from the group consisting of —CH₂—, —CH(lower alkyl)- and —C(dilower alkyl)-;
R is —OR⁶, —O(CO)R⁶, —O(CO)OR⁹ or —O(CO)NR⁶R⁷; R¹ is hydrogen, lower alkyl or aryl; or R and R¹ together are ═O;
q is 0 or 1;
r is 0, 1 or 2;
m and n are independently 0, 1, 2, 3, 4 or 5; provided that the sum of m, n and q is 1, 2, 3, 4 or 5;
R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;
R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, —CF₃, —CN, —NO₂, halogen, -(lower alkylene)COOR⁶ and —CH═CH—COOR⁶;
R⁶, R⁷ and R⁸ are independently selected from the group consisting of hydrogen, lower alkyl, aryl and aryl-substituted lower alkyl;
R⁹ is lower alkyl, aryl or aryl-substituted lower alkyl; and
R¹⁰ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, —S(O)₀₋₂R⁹, —O(CH₂)₁₋₁₀—COOR⁶, —O(CH₂)₁₋₁₀CONR⁶R⁷, —CF₃, —CN, —NO₂ and halogen.

8. The method according to claim 2, wherein the sterol or 5-α-stanol absorption inhibitor is a compound of formula: 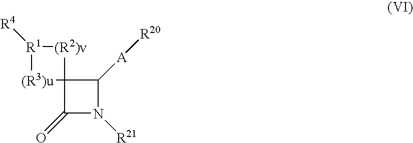
or a pharmaceutically acceptable salt thereof or a solvate thereof,
wherein, in formula (VI):
R¹ is 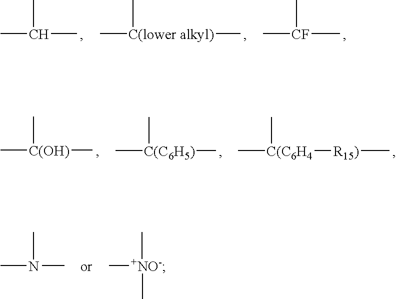
R² and R³ are independently selected from the group consisting of:
—CH₂—, —CH(lower alkyl)-, —C(di-lower alkyl)-, —CH═CH— and —C(lower alkyl)═CH—; or R¹ together with an adjacent R², or R¹ together with an adjacent R³, form a —CH═CH— or a —CH═C(lower alkyl)- group;
u and v are independently 0, 1, 2 or 3, provided both are not zero; provided that when R² is —CH═CH— or —C(lower alkyl)═CH—, v is 1; provided that when R³ is —CH═CH— or —C(lower alkyl)═CH—, u is 1; provided that when v is 2 or 3, the R²'s can be the same or different; and provided that when u is 2 or 3, the R³'s can be the same or different;
R⁴ is selected from B—(CH₂)ₘC(O)—, wherein m is 0, 1, 2, 3, 4 or 5;
B—(CH₂)_{q}—, wherein q is 0, 1, 2, 3, 4, 5 or 6; B—(CH₂)ₑ-Z-(CH₂)ᵣ—, wherein Z is —O—, —C(O)—, phenylene, —N(R⁸)— or —S(O)₀₋₂—, e is 0, 1, 2, 3, 4 or 5 and r is 0, 1, 2, 3, 4 or 5, provided that the sum of e and r is 0, 1, 2, 3, 4, 5 or 6; B—(C₂-C₆ alkenylene)—; B—(C₄-C₆ alkadienylene); B—(CH₂)ₜ-Z-(C₂-C₆ alkenylene)-, wherein Z is as defined above, and wherein t is 0, 1, 2 or 3, provided that the sum of t and the number of carbon atoms in the alkenylene chain is 2, 3, 4, 5 or 6; B—(CH₂)_{f}—V—(CH₂)_{g}—, wherein V is C₃-C₆ cycloalkylene, f is 1, 2, 3, 4 or 5 and g is 0, 1, 2, 3, 4 or 5, provided that the sum of f and g is 1, 2, 3, 4, 5 or 6; B—(CH₂)ₜ—V—(C₂-C₆ alkenylene)- or B—(C₂-C₆ alkenylene)-V—(CH₂)ₜ—, wherein V and t are as defined above, provided that the sum of t and the number of carbon atoms in the alkenylene chain is 2, 3, 4, 5 or 6;
B—(CH₂)ₐ-Z-(CH₂)_{b}—V—(CH₂)_{d}—, wherein Z and V are as defined above and a, b and d are independently 0, 1, 2, 3, 4, 5 or 6, provided that the sum of a, b and d is 0, 1, 2, 3, 4, 5 or 6; or T-(CH₂)ₛ—, wherein T is cycloalkyl of 3-6 carbon atoms and s is 0, 1, 2, 3, 4, 5 or 6; or
R¹ and R⁴ together form the group 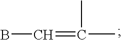
B is selected from indanyl, indenyl, naphthyl, tetrahydronaphthyl, heteroaryl or W-substituted heteroaryl, wherein heteroaryl is selected from the group consisting of pyrrolyl, pyridinyl, pyrimidinyl, pyrazinyl, triazinyl, imidazolyl, thiazolyl, pyrazolyl, thienyl, oxazolyl and furanyl, and for nitrogen-containing heteroaryls, the N-oxides thereof, or 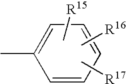
W is 1 to 3 substituents independently selected from the group consisting of lower alkyl, hydroxy lower alkyl, lower alkoxy, alkoxyalkyl, alkoxyalkoxy, alkoxycarbonylalkoxy, (lower alkoxyimino)-lower alkyl, lower alkanedioyl, lower alkyl lower alkanedioyl, allyloxy, —CF₃, —OCF₃, benzyl, R⁷-benzyl, benzyloxy, R⁷-benzyloxy, phenoxy, R⁷-phenoxy, dioxolanyl, NO₂, —N(R⁸)(R⁹), N(R⁸)(R⁹)-lower alkylene-, N(R⁸)(R⁹)-lower alkylenyloxy-, OH, halogeno, —CN, —N₃, —NHC(O)OR¹⁰, —NHC(O)R¹⁰, R¹¹O₂SNH—, (R¹¹O₂S)₂N—, —S(O)₂NH₂, —S(O)₀₋₂R⁸, tert-butyldimethyl-silyloxymethyl, —C(O)R¹², —COOR¹⁹, —CON(R⁸)(R⁹), —CH═CHC(O)R¹², -lower alkylene-C(O)R¹²,
R¹⁰C(O)(lower alkylenyloxy)—, N(R⁸)(R⁹)C(O)(lower alkylenyloxy)- and 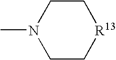
for substitution on ring carbon atoms,
and the substituents on the substituted heteroaryl ring nitrogen atoms, when present, are selected from the group consisting of lower alkyl, lower alkoxy, —C(O)OR¹⁰, —C(O)R¹⁰, OH, N(R⁸)(R⁹)-lower alkylene-, N(R⁸)(R⁹)-lower alkylenyloxy-, —S(O)₂NH₂ and 2-(trimethylsilyl)-ethoxymethyl;
R⁷ is 1-3 groups independently selected from the group consisting of lower alkyl, lower alkoxy, —COOH, NO₂, —N(R⁸)(R⁹), OH, and halogeno;
R⁸ and R⁹ are independently selected from H or lower alkyl;
R¹⁰ is selected from lower alkyl, phenyl, R⁷-phenyl, benzyl or R⁷-benzyl;
R¹¹ is selected from OH, lower alkyl, phenyl, benzyl, R⁷-phenyl or R⁷-benzyl;
R¹² is selected from H, OH, alkoxy, phenoxy, benzyloxy, 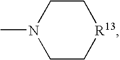
—N(R⁸)(R⁹), lower alkyl, phenyl or R⁷-phenyl;
R¹³ is selected from —O—, —CH₂—, —NH—, —N(lower alkyl)- or —NC(O)R¹⁹;
R¹⁵, R¹⁶ and R¹⁷ are independently selected from the group consisting of H and the groups defined for W; or R¹⁵ is hydrogen and R¹⁶ and R¹⁷, together with adjacent carbon atoms to which they are attached, form a dioxolanyl ring;
R¹⁹ is H, lower alkyl, phenyl or phenyl lower alkyl; and
R²⁰ and R²¹ are independently selected from the group consisting of phenyl, W-substituted phenyl, naphthyl, W-substituted naphthyl, indanyl, indenyl, tetrahydronaphthyl, benzodioxolyl, heteroaryl, W-substituted heteroaryl, benzofused heteroaryl, W-substituted benzofused heteroaryl and cyclopropyl, wherein heteroaryl is as defined above.

9. The method according to claim 2, wherein the sterol or 5-α-stanol absorption inhibitor is a compound of formula by formulae (VIIA) and (VIIB): 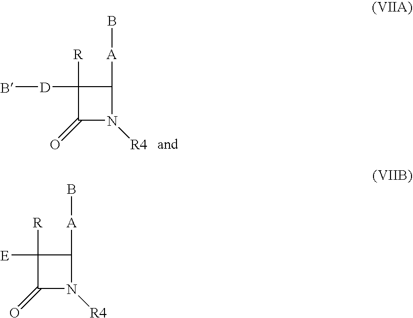
or a pharmaceutically acceptable salt or solvate thereof,
wherein in formulae (VIIA) or (VIIB):
A is —CH═CH—, —C≡C— or —(CH₂)ₚ— wherein p is 0, 1 or 2;
B is 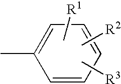
B′ is 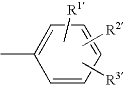
D is —(CH₂)ₘC(O)— or —(CH₂)_{q}— wherein m is 1, 2, 3 or 4 and q is 2, 3 or 4;
E is C₁₀ to C₂₀ alkyl or —C(O)—(C₉ to C₁₉)-alkyl, wherein the alkyl is straight or branched, saturated or containing one or more double bonds;
R is hydrogen, C₁-C₁₅ alkyl, straight or branched, saturated or containing one or more double bonds, or B—(CH₂)ᵣ—, wherein r is 0, 1, 2, or 3;
R¹, R², R³, R^{1′}, R^{2′}, and R^{3′} are independently selected from the group consisting of hydrogen, lower alkyl, lower alkoxy, carboxy, NO₂, NH₂, OH, halogeno, lower alkylamino, dilower alkylamino, —NHC(O)OR⁵, R⁶O₂SNH— and —S(O)₂NH₂;
R⁴ is 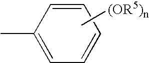
wherein n is 0, 1, 2 or 3;
R⁵ is lower alkyl; and
R⁶ is OH, lower alkyl, phenyl, benzyl or substituted phenyl wherein the substituents are 1-3 groups independently selected from the group consisting of lower alkyl, lower alkoxy, carboxy, NO₂, NH₂, OH, halogeno, lower alkylamino and dilower alkylamino.

10. The method according to claim 2, wherein the sterol or 5-α-stanol absorption inhibitor is a compound of formula (VIII): 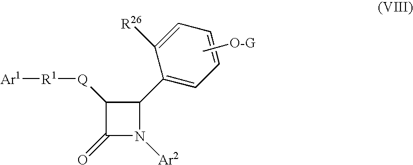
or a pharmaceutically acceptable salt thereof or a solvate thereof,
wherein, in formula (VIII) above,
R²⁶ is H or OG¹;
G and G¹ are independently selected from the group consisting of H, 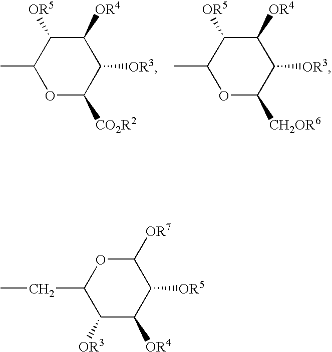
and 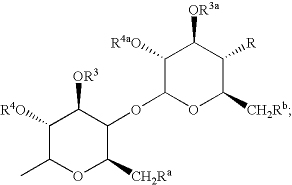
provided that when R²⁶ is H or OH, G is not H;
R, R^{a} and R^{b} are independently selected from the group consisting of H, —OH, halogeno, —NH₂, azido, (C₁-C₆)alkoxy(C₁-C₆)-alkoxy or —W—R³⁰;
W is independently selected from the group consisting of —NH—C(O)—, —O—C(O)—, —O—C(O)—N(R³¹)—, —NH—C(O)—N(R³¹)— and —O—C(S)—N(R³¹)—;
R² and R⁶ are independently selected from the group consisting of H, (C₁-C₆)alkyl, aryl and aryl(C₁-C₆)alkyl;
R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;
R³⁰ is selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;
R³¹ is selected from the group consisting of H and (C₁-C₄)alkyl;
T is selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;
R³² is independently selected from 1-3 substituents independently selected from the group consisting of halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;
Ar¹ is aryl or R¹⁰-substituted aryl;
Ar² is aryl or R¹¹-substituted aryl;
Q is a bond or, with the 3-position ring carbon of the azetidinone, forms the spiro group 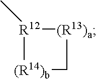
and
R¹ is selected from the group consisting of
—(CH₂)_{q}—, wherein q is 2-6, provided that when Q forms a spiro ring, q can also be zero or 1;
—(CH₂)ₑ-E-(CH₂)ᵣ—, wherein E is —O—, —C(O), phenylene, —NR²²— or —S(O)₀₋₂—, e is 0-5 and r is 0-5, provided that the sum of e and r is 1-6;
—(C₂-C₆)alkenylene-; and
—(CH₂)_{f}—V—(CH₂)_{g}—, wherein V is C₃-C₆ cycloalkylene, f is 1-5 and g is 0-5, provided that the sum of f and g is 1-6;
R¹² is 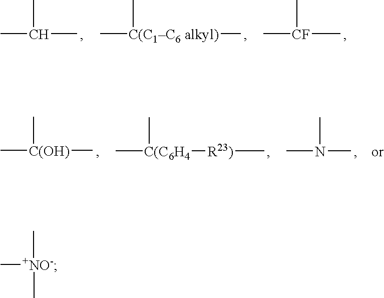
R¹³ and R¹⁴ are independently selected from the group consisting of
—CH₂—, —CH(C₁-C₆ alkyl)-, —C(di-(C₁-C₆)alkyl), —CH═CH— and —C(C₁-C₆ alkyl)═CH—; or R¹² together with an adjacent R¹³, or R¹² together with an is adjacent R¹⁴, form a —CH═CH— or a —CH═C(C₁-C₆ alkyl)- group;
a and b are independently 0, 1, 2 or 3, provided both are not zero;
provided that when R¹³ is —CH═CH— or —C(C₁-C₆ alkyl)═CH—, a is 1;
provided that when R¹⁴ is —CH═CH— or —C(C₁-C₆ alkyl)═CH—, b is 1;
provided that when a is 2 or 3, the R¹³'s can be the same or different; and
provided that when b is 2 or 3, the R¹⁴'s can be the same or different;
and when Q is a bond, R¹ also can be: 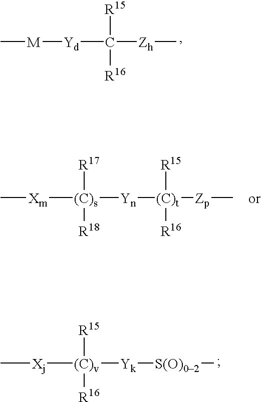
M is —O—, —S—, —S(O)— or —S(O)₂—;
X, Y and Z are independently selected from the group consisting of —CH₂—, —CH(C₁-C₆)alkyl- and —C(di-(C₁-C₆)alkyl);
R¹⁰ and R¹¹ are independently selected from the group consisting of 1-3 substituents independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)-COOR¹⁹, —CH═CH—COOR¹⁹, —CF₃, —CN, —NO₂ and halogen;
R¹⁵ and R¹⁷ are independently selected from the group consisting of —OR¹⁹, —O(CO)R¹⁹, —O(CO)OR²¹ and —O(CO)NR¹⁹R²⁰;
R¹⁶ and R¹⁸ are independently selected from the group consisting of H, (C₁-C₆)alkyl and aryl; or R¹⁵ and R¹⁶ together are ═O, or R¹⁷ and R¹⁸ together are ═O;
d is 1, 2 or 3;
h is 0, 1, 2, 3 or 4;
s is 0 or 1; t is 0 or 1; m, n and p are independently 0-4;
provided that at least one of s and t is 1, and the sum of m, n, p, s and t is 1-6;
provided that when p is 0 and t is 1, the sum of m, s and n is 1-5; and provided that when p is 0 and s is 1, the sum of m, t and n is 1-5;
v is 0 or 1;
j and k are independently 1-5, provided that the sum of j, k and v is 1-5;
and when Q is a bond and R¹ is 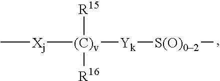
Ar¹ can also be pyridyl, isoxazolyl, furanyl, pyrrolyl, thienyl, imidazolyl, pyrazolyl, thiazolyl, pyrazinyl, pyrimidinyl or pyridazinyl;
R¹⁹ and R²⁰ are independently selected from the group consisting of H, (C₁-C₆)alkyl, aryl and aryl-substituted (C₁-C₆)alkyl;
R²¹ is (C₁-C₆)alkyl, aryl or R²⁴-substituted aryl;
R²² is H, (C₁-C₆)alkyl, aryl (C₁-C₆)alkyl, —C(O)R¹⁹ or —COOR¹⁹;
R²³ and R²⁴ are independently 1-3 groups independently selected from the group consisting of H, (C₁-C₆)alkyl, (C₁-C₆)alkoxy, —COOH, NO₂, —NR¹⁹R²⁰, —OH and halogeno; and
R²⁵ is H, —OH or (C₁-C₆)alkoxy.

11. The method according to claim 2, wherein the sterol or 5-α-stanol absorption inhibitor is a compound of formula (IX): 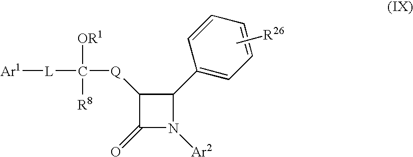
or a pharmaceutically acceptable salt or solvate thereof,
wherein in formula (IX):
R¹ is selected from the group consisting of H, G, G¹, G², —SO₃H and —PO₃H;
G is selected from the group consisting of: H, 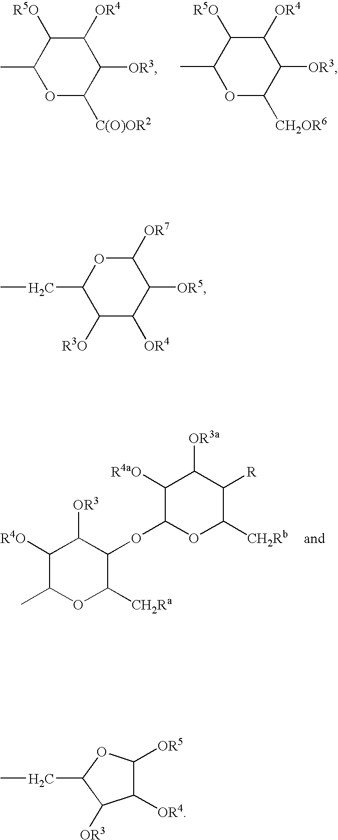
(sugar derivatives)
wherein R⁶, R^{a} and R^{b} are each independently selected from the group consisting of H, —OH, halo, —NH₂, azido, (C₁-C₆)alkoxy(C₁-C₆)alkoxy or —W—R³⁰;
W is independently selected from the group consisting of —NH—C(O), —O—C(O)—, —O—C(O)—N(R³¹), —NH—C(O)—N(R³¹)— and —O—C(S)—N(R³¹)—;
R² and R⁶ are each independently selected from the group consisting of H, (C₁-C₆)alkyl, acetyl, aryl and aryl(C₁-C₆)alkyl;
R³, R⁴, R⁵, R⁷, R^{3a} and R^{4a} are each independently selected from the group consisting of H, (C₁-C₆)alkyl, acetyl, aryl(C₁-C₆)alkyl, —C(O)(C₁-C₆)alkyl and —C(O)aryl;
R³⁰ is independently selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;
R³¹ is independently selected from the group consisting of H and (C₁-C₄)alkyl;
T is independently selected from the group consisting of phenyl, furyl, thienyl, pyrrolyl, oxazolyl, isoxazolyl, thiazolyl, isothiazolyl, benzothiazolyl, thiadiazolyl, pyrazolyl, imidazolyl and pyridyl;
R³² is independently selected from 1-3 substituents which are each independently selected from the group consisting of H, halo, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;
G¹ is represented by the structure: 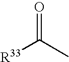
wherein R³³ is independently selected from the group consisting of unsubstituted alkyl, R³⁴-substituted alkyl, (R³⁵)(R³⁶)alkyl-, 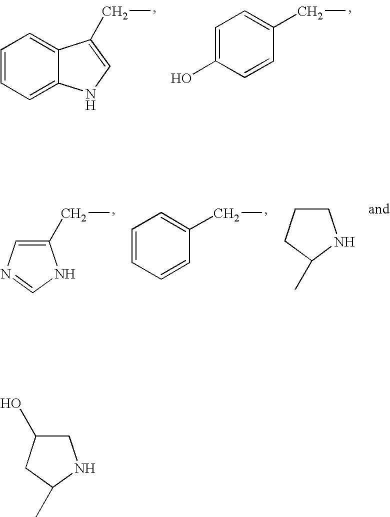
R³⁴ is one to three substituents, each R³⁴ being independently selected from the group consisting of HOOC—, HO—, HS—, (CH₃)S—, H₂N—, (NH₂)(NH)C(NH)—, (NH₂)C(O)— and HOOCCH(NH₃⁺)CH₂SS—;
R³⁵ is independently selected from the group consisting of H and NH₂—;
R³⁶ is independently selected from the group consisting of H, unsubstituted alkyl, R³⁴-substituted alkyl, unsubstituted cycloalkyl and R³⁴-substituted cycloalkyl;
G² is represented by the structure: 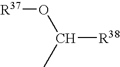
wherein R³⁷ and R³⁸ are each independently selected from the group consisting of (C₁-C₆)alkyl and aryl;
R²⁶ is one to five substituents, each R²⁶ being independently selected from the group consisting of:
a) H;
d) —OH;
e) —OCH₃;
d) fluorine;
e) chlorine;
k) —O-G¹;
l) —O-G²;
m) —SO₃H; and
n) —PO₃H;
provided that when R¹ is H, R²⁶ is not H, —OH, —OCH₃ or —O-G;
Ar¹ is aryl, R¹⁰-substituted aryl, heteroaryl or R¹⁰-substituted heteroaryl;
Ar² is aryl, R¹¹-substituted aryl, heteroaryl or R¹¹-substituted heteroaryl;
L is selected from the group consisting of:
f) a covalent bond;
g) —(CH₂)_{q}—, wherein q is 1-6;
h) —(CH₂)ₑ-E-(CH₂)ᵣ—, wherein E is —O—, —C(O)—, phenylene, —NR²²— or —S(O)₀₋₂—, e is 0-5 and r is 0-5, provided that the sum of e and r is 1-6;
i) —C₂-C₆)alkenylene-;
j) —(CH₂)_{f}—V—(CH₂)_{g}—, wherein V is C₃-C₆cycloalkylene, f is 1-5 and g is 0-5, provided that the sum of f and g is 1-6; and f) 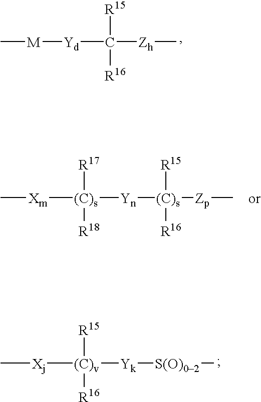
wherein M is —O—, —S—, —S(O)— or —S(O)₂—;
X, Y and Z are each independently selected from the group consisting of —CH₂—, —CH(C₁-C₆)alkyl- and —C(di-(C₁-C₆)alkyl)-;
R⁸ is selected from the group consisting of H and alkyl;
R¹⁰ and R¹¹ are each independently selected from the group consisting of 1-3 substituents which are each independently selected from the group consisting of (C₁-C₆)alkyl, —OR¹⁹, —O(CO)R¹⁹, —O(CO)OR²¹, —O(CH₂)₁₋₅OR¹⁹, —O(CO)NR¹⁹R²⁰, —NR¹⁹R²⁰, —NR¹⁹(CO)R²⁰, —NR¹⁹(CO)OR²¹, —NR¹⁹(CO)NR²⁰R²⁵, —NR¹⁹SO₂R²¹, —COOR¹⁹, —CONR¹⁹R²⁰, —COR¹⁹, —SO₂NR¹⁹R²⁰, S(O)₀₋₂R²¹, —O(CH₂)₁₋₁₀—COOR¹⁹, —O(CH₂)₁₋₁₀CONR¹⁹R²⁰, —(C₁-C₆ alkylene)COOR¹⁹, —CH═CH—COOR¹⁹, —CF₃, —CN, —NO₂ and halo;
R¹⁵ and R¹⁷ are each independently selected from the group consisting of —OR¹⁹, —OC(O)R¹⁹, —OC(O)OR²¹, —OC(O)NR¹⁹R²⁰;
R¹⁶ and R¹⁸ are each independently selected from the group consisting of H, (C₁-C₆)alkyl and aryl;
or R¹⁵ and R¹⁶ together are ═O, or R¹⁷ and R¹⁸ together are ═O;
d is 1, 2 or 3;
h is 0, 1, 2, 3 or 4;
s is 0 or 1;
t is 0 or 1;
m, n and p are each independently selected from 0-4;
provided that at least one of s and t is 1, and the sum of m, n, p, s and t is 1-6; provided that when p is 0 and t is 1, the sum of m, n and p is 1-5; and provided that when p is 0 and s is 1, the sum of m, t and n is 1-5;
v is 0 or 1;
j and k are each independently 1-5, provided that the sum of j, k and v is 1-5;
Q is a bond, —(CH₂)_{q}—, wherein q is 1-6, or, with the 3-position ring carbon of the azetidinone, forms the spiro group 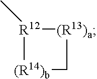
wherein R¹² is 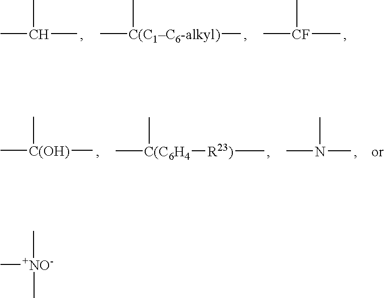
R¹³ and R¹⁴ are each independently selected from the group consisting of —CH₂—, —CH(C₁-C₆ alkyl)-, —C(di-(C₁-C₆)alkyl), —CH═CH— and —C(C₁-C₆ alkyl)═CH—; or R¹² together with an adjacent R¹³, or R¹² together with an adjacent R¹⁴, form a —CH═CH— or a —CH═C(C₁-C₆ alkyl)- group;
a and b are each independently 0, 1, 2 or 3, provided both are not zero; provided that when R¹³ is —CH═CH— or —C(C₁-C₆ alkyl)═CH—, a is 1; provided that when R¹⁴ is —CH═CH— or —C(C₁-C₆ alkyl)═CH—, b is 1; provided that when a is 2 or 3, the R¹³'s can be the same or different; and provided that when b is 2 or 3, the R¹⁴'s can be the same or different;
and when Q is a bond and L is 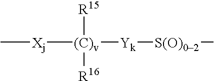
then Ar¹ can also be pyridyl, isoxazolyl, furanyl, pyrrolyl, thienyl, imidazolyl, pyrazolyl, thiazolyl, pyrazinyl, pyrimidinyl or pyridazinyl;
R¹⁹ and R²⁰ are each independently selected from the group consisting of H, (C₁-C₆)alkyl, aryl and aryl-substituted (C₁-C₆)alkyl;
R²¹ is (C₁-C₆)alkyl, aryl or R²⁴-substituted aryl;
R²² is H, (C₁-C₆)alkyl, aryl(C₁-C₆)alkyl, —C(O)R¹⁹ or —COOR¹⁹;
R²³ and R²⁴ are each independently selected from the group consisting of 1-3 substituents which are each independently selected from the group consisting of H, (C₁-C₆)alkyl, (C₁-C₆)alkoxy, —COOH, NO₂, —NR¹⁹R²⁰, —OH and halo; and
R²⁵ is H, —OH or (C₁-C₆)alkoxy.

12. The method according to claim 2, wherein the H₃ receptor antagonist/inverse agonist is of the imidazole type.

13. The method according to claim 2, wherein the H₃ receptor antagonist/agonist is a compound of formula (XIII) 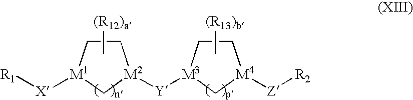
or a pharmaceutically acceptable salt or solvate thereof,
wherein in formula (XIII):
(1) R₁ is selected from:
(a) aryl;
(b) heteroaryl;
(c) heterocycloalkyl
(d) alkyl;
(e) cycloalkyl; or
f) alkylaryl;
wherein said R₁ groups are optionally substituted with 1 to 4 substituents independently selected from:
(1) halogen;
(2) hydroxyl;
(3) lower alkoxy;
(4) —CF₃;
(5) CF₃O—;
(6) —NR₄R₅;
(7) phenyl;
(8) —NO₂,
(9) —CO₂R₄;
(10) —CON(R₄)₂ wherein each R₄ is the same or different;
(11) —S(O)_{m′}N(R₂₀)₂ wherein each R₂₀ is the same or different H or alkyl group;
(12) —CN; or
(13) alkyl; or
(2) R₁ and X′ taken together form a group selected from: 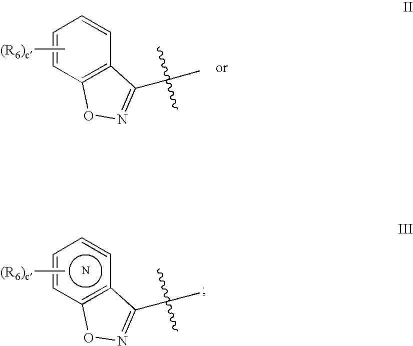
(3) X′ is selected from: ═C(O), ═C(NOR₃), ═C(NNR₄R₅), 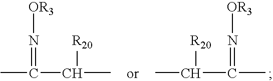
(4) M¹ is carbon;
(5) M² is selected from C or N;
(6) M³ and M⁴ are independently selected from C or N;
(7) Y′ is selected from: is —CH₂—, ═C(O), ═C(NOR₂₀) (wherein R₂₀ is as defined above), or ═C(S);
(8) Z′ is a C₁-C₆ alkyl group;
(9) R₂ is a five or six-membered heteroaryl ring, said six-membered heteroaryl ring comprising 1 or 2 nitrogen atoms with the remaining ring atoms being carbon, and said five-membered heteroaryl ring containing 1 or 2 heteroatoms selected from: nitrogen, oxygen, or sulfur with the remaining ring atoms being carbon; said five or six membered heteroaryl rings being optionally substituted with 1 to 3 substituents independently selected from: halogen, hydroxyl, lower alkyl, lower alkoxy, —CF₃, CF₃O—, —NR₄R₅, phenyl, —NO₂, —CO₂R₄, —CON(R₄)₂ wherein each R₄ is the same or different, —CH₂NR₄R₅, —(N)C(NR₄R₅)₂, or —CN;
(10) R₃ is selected from:
(a) hydrogen;
(b) C₁-C₆ alkyl;
(c) aryl;
(d) heteroaryl;
(e) heterocycloalkyl;
(f) arylalkyl;
(g) —(CH₂)_{e′}—C(O)N(R₄)₂ wherein each R₄ is the same or different,
(h) —(CH₂)_{e′}—C(O)OR₄;
(i) —(CH₂)_{e′}—C(O)R₃₀ wherein R₃₀ is a heterocycloalkyl group;
(j) —CF₃; or
(k) —CH₂CF₃;
wherein said aryl, heteroaryl, heterocycloalkyl, and the aryl portion of said arylalkyl are optionally substituted with 1 to 3 substituents selected from: halogen, —OH, —OCF₃, —CF₃, —CN, —N(R₄₅)₂, —CO₂R₄₅, or —C(O)N(R₄₅)₂, wherein each R₄₅ is independently selected from: H, alkyl, alkylaryl, or alkylaryl wherein said aryl moiety is substituted with 1 to 3 substituents independently selected from —CF₃, —OH, halogen, alkyl, —NO₂, or —CN;
(11) R₄ is selected from: hydrogen, C₁-C₆ alkyl, aryl, alkylaryl, said aryl and alkylaryl groups being optionally substituted with 1 to 3 substituents selected from: halogen, —CF₃, —OCF₃, —OH, —N(R₄₅)₂, —CO₂R₄₅, —C(O)N(R₄₅)₂, or —CN; wherein R₄₅ is as defined above;
(12) R₅ is selected from: hydrogen, C₁-C₆ alkyl, —C(O)R₄, —C(O)₂R₄, or —C(O)N(R₄)₂ wherein each R₄ is independently selected, and R₄ is as defined above;
(13) or R₄ and R₅ taken together with the nitrogen atom to which they are bound forms a five or six membered heterocycloalkyl ring;
(14) R₆ is selected from: alkyl, aryl, alkylaryl, halogen, hydroxyl, lower alkoxy, —CF₃, CF₃O—, —NR₄R₅, phenyl, —NO₂, —CO₂R₅, —CON(R₄)₂ wherein each R₄ is the same or different, or —CN;
(15) R₁₂ is selected from: alkyl, hydroxyl, alkoxy, or fluoro;
(16) R₁₃ is selected from: alkyl, hydroxyl, alkoxy, or fluoro;
(17) a′ (subscript for R₁₂) is 0 to 2;
(18) b′ (subscript for R₁₂) is 0 to 2;
(19) c′ (subscript for R₆) is 0 to 2;
(20) e′ is 0 to 5;
(21) m′ is 1 or 2;
(22) n′ is 1, 2 or 3; and
(23) p′ is 1, 2 or 3, with the proviso that when M³ and M⁴ are both nitrogen, then p′ is 2 or 3 (i.e., p is not 1 when M³ and M² are both nitrogen).

14. The method according to claim 13, wherein the H₃ receptor antagonist/agonist is a compound selected from the group consisting of:

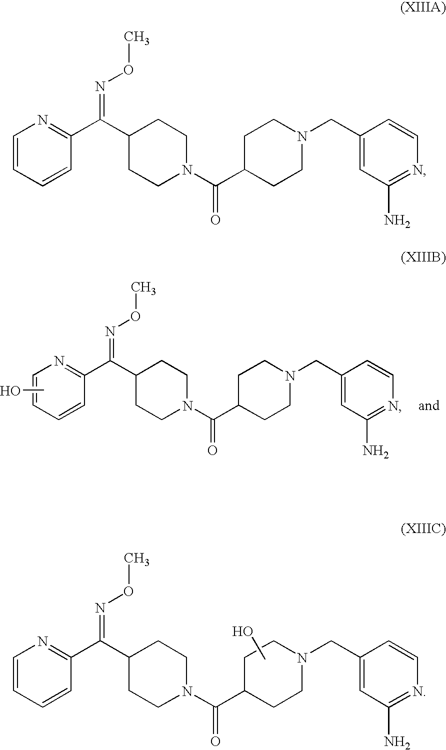

15. The method according to claim 13, wherein the sterol or 5-α-stanol absorption inhibitor is a compound of formula (I): 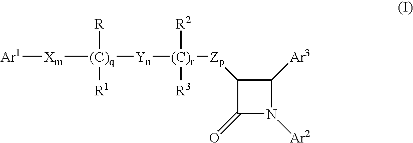
or pharmaceutically acceptable salts or solvates thereof,
wherein in formula (I):
Ar¹ and Ar² are independently selected from the group consisting of aryl and R⁴-substituted aryl;
Ar⁴ is aryl or R⁵-substituted aryl;
X, Y and Z are independently selected from the group consisting of —CH₂—, —CH(lower alkyl)- and —C(dilower alkyl)-;
R and R² are independently selected from the group consisting of —OR⁶, —O(CO)R⁶, —O(CO)OR⁹ and —O(CO)NR⁶R⁷;
R¹ and R³ are independently selected from the group consisting of hydrogen, lower alkyl and aryl;
q is 0 or 1; r is 0 or 1; m, n and p are independently selected from 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5;
R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶, —CH═CH—COOR⁶, —CF₃, —CN, —NO₂ and halogen;
R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶ and —CH═CH—COOR⁶;
R⁶, R⁷ and R⁸ are independently selected from the group consisting of hydrogen, lower alkyl, aryl and aryl-substituted lower alkyl; and
R⁹ is lower alkyl, aryl or aryl-substituted lower alkyl.

16. The method according to claim 14, wherein the sterol or 5-α-stanol absorption inhibitor is a compound of formula (VIA) 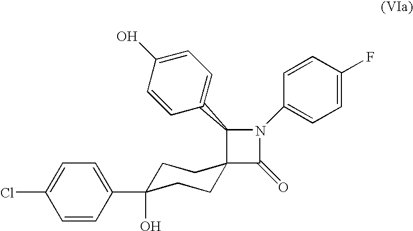
or pharmaceutically acceptable salts or solvates thereof.

17. The method according to claim 14, wherein the sterol or 5-α-stanol absorption inhibitor is a compound of formula (II): 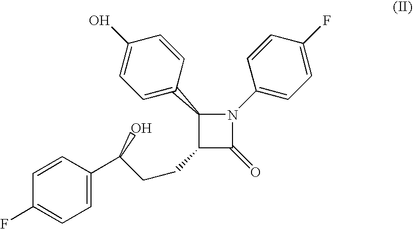
or pharmaceutically acceptable salts or solvates thereof.

18. The method according to claim 15, which further comprises an effective amount of an HMG-CoA reductase inhibitor.

19. The method according to claim 18, wherein the HMG-CoA reductase inhibitor is selected from the group consisting of lovastatin, pravastatin fluvastatin, simvastatin, atorvastatin, cerivastatin, pitavastatin, and rosuvastatin.

20. The method according to claim 17, which further comprises an effective amount of an HMG-CoA reductase inhibitor wherein said inhibitor is simvastatin.

21. The method according to claim 15, which further comprises as a third active component a PPAR activator, nicotinic acid and/or a nicotinic acid receptor agonist or a bile acid sequestrant.

22. The method according to claim 21, wherein the third active component is cholestyramine, colestipol, clofibrate, gemfibrozil, fenofibrate, or niacin.

23. The method according to claim 2, wherein the H₃ receptor antagonist/agonist is a compound of formula (XIV) 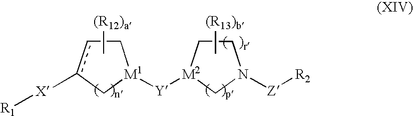
or a pharmaceutically acceptable salt or solvate thereof,
wherein, in formula (XIV):
the dotted line represents an optional double bond;
a′ is 0 to 2;
b′ is 0 to 2;
n′ is 1, 2 or 3;
p′ is 1, 2 or 3;
r′ is 0, 1, 2, or 3;
with the provisos that when M² is N, p′ is not 1; and that when r′ is 0, M² is C(R₃); and that the sum of p′ and r′ is 1 to 4;
M¹ is C(R₃) or N;
M² is C(R₃) or N;
X′ is a bond or C₁-C₆ alkylene;
Y′ is —C(O)—, —C(S)—, —(CH₂)_{q′}—, —NR₄C(O)—, —C(O)NR₄—, —C(O)CH₂—, —SO₂—, —N(R₄)—, —NH—C(═N—CN)— or —C(═N—CN)—NH—; with the provisos that when M¹ is N, Y′ is not —NR₄C(O)— or —NH—C(═N—CN)—; when M² is N, Y′ is not —C(O)NR₄— or —C(═N—CN)—NH—; and when Y′ is —N(R₄)—, M¹ is CH and M² is C(R₃);
q′ is 1 to 5, provided that when both M¹ and M² are N, q′ is 2 to 5;
Z′ is a bond, C₁-C₆ alkylene, C₁-C₆ alkenylene, —C(O)—, —CH(CN)—, —SO₂— or —CH₂C(O)NR₄—;
R₁ is 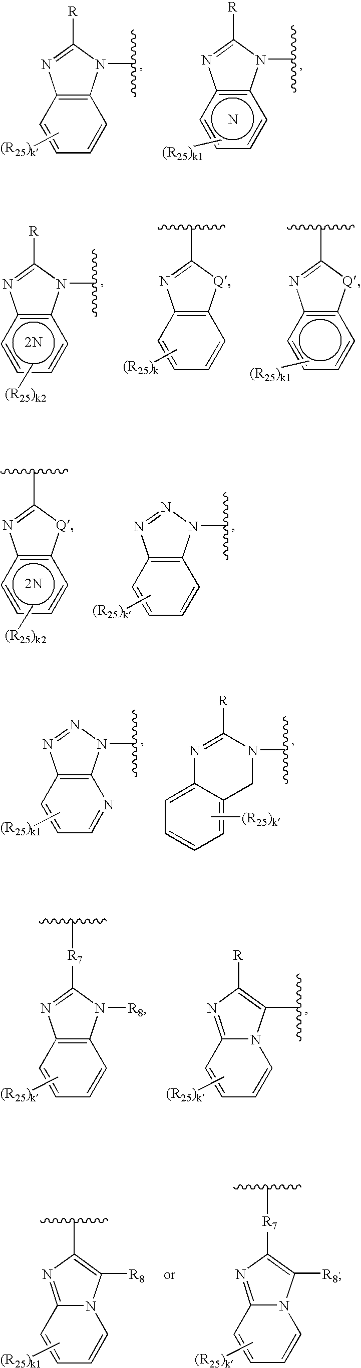
Q′ is —N(R₈)—, —S— or —O—;
k′ is 0, 1, 2, 3 or 4;
k1 is 0, 1, 2 or 3;
k2 is 0, 1 or 2;
R is H, C₁-C₆ alkyl, halo(C₁-C₆)alkyl-, C₁-C₆ alkoxy, (C₁-C₆)alkoxy-(C₁-C₆)alkyl-, (C₁-C₆)-alkoxy-(C₁-C₆)alkoxy, (C₁-C₆)alkoxy-(C₁-C₆)alkyl-SO₀₋₂, R₃₂-aryl(C₁-C₆)alkoxy-, R₃₂-aryl(C₁-C₆)alkyl-, R₃₂-aryl, R₃₂-aryloxy, R₃₂-heteroaryl, (C₃-C₆)cycloalkyl, (C₃-C₆)cycloalkyl-(C₁-C₆)alkyl, (C₃-C₆)cycloalkyl-(C₁-C₆)alkoxy, (C₃-C₆)cycloalkyl-oxy-, R₃₇-heterocycloalkyl, R₃₇-heterocycloalkyl-oxy-, R₃₇-heterocycloalkyl-(C₁-C₆)alkoxy, N(R₃₀)(R₃₁)—(C₁-C₆)alkyl-, —N(R₃₀)(R₃₁), —NH—(C₁-C₆)alkyl-O—(C₁-C₆)alkyl, —NHC(O)NH(R₂₉); R₂₉—S(O)₀₂—, halo(C₁-C₆)alkyl-S(O)₀₋₂—, N(R₃₀)(R₃₁)—(C₁-C₆)alkyl-S(O)₀₋₂— or benzoyl;
R₈ is H, C₁-C₆ alkyl, halo(C₁-C₆)alkyl-, (C₁-C₆)alkoxy-(C₁-C₆)alkyl-, R₃₂-aryl(C₁-C₆)alkyl-, R₃₂-aryl, R₃₂-heteroaryl, (C₃-C₆)cycloalkyl, (C₃-C₆)cycloalkyl-(C₁-C₆)alkyl, R₃₇-heterocycloalkyl, N(R₃₀)(R₃₁)—(C₁-C₆)alkyl-, R₂₉—S(O)₂—, halo(C₁-C₆)alkyl-S(O)₂—, R₂₉—S(O)₀₋₁—(C₂-C₆)alkyl-, halo(C₁-C₆)alkyl-S(O)₀₋₁—(C₂-C₆)alkyl-;
R₃ is a six-membered heteroaryl ring having 1 or 2 heteroatoms independently selected from N or N—O, with the remaining ring atoms being carbon; a five-membered heteroaryl ring having 1, 2, 3 or 4 heteroatoms independently selected from N, O or S, with the remaining ring atoms being carbon; R₃₂-quinolyl; R₃₂-aryl; heterocycloalkyl; (C₃-C₆)cycloalkyl; C₁-C₆ alkyl; hydrogen; thianaphthenyl; 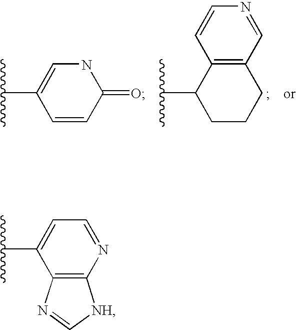
wherein said six-membered heteroaryl ring or said five-membered heteroaryl ring is optionally substituted by R₆;
R₃ is H, halogen, C₁-C₆ alkyl, —OH, (C₁-C₆)alkoxy or —NHSO₂-(C₁-C₆)alkyl;
R₄ is independently selected from the group consisting of hydrogen, C₁-C₆ alkyl, C₃-C₆ cycloalkyl, (C₃-C₆)cycloalkyl(C₁-C₆)alkyl, R₃₃-aryl, R₃₃-aryl(C₁-C₆)alkyl, and R₃₂-heteroaryl;
R⁵ is hydrogen, C₁-C₆ alkyl, —C(O)R²⁰, —C(O)₂R²⁰, C(O)N(R²⁰)₂, (C₁-C₆)alkyl-SO₂—, or (C₁-C₆)alkyl-SO₂—NH—;
or R₄ and R₅, together with the nitrogen to which they are attached, form an azetidinyl, pyrrolidinyl, piperidinyl, piperazinyl or morpholinyl ring;
R₆ is 1 to 3 substituents independently selected from the group consisting of —OH, halogen, C₁-C₆ alkyl-, C₁-C₆ alkoxy, C₁-C₆ alkylthio, —CF₃, —NR₄R₅, —CH₂—NR₄R₅, —NHSO₂R₂₂, —N(SO₂R₂₂)₂, phenyl, R₃₃-phenyl, NO₂, —CO₂R₄, —CON(R₄)₂, 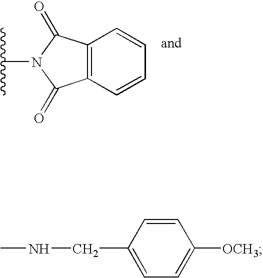
R₇ is —N(R₂₉)—, —O— or —S(O)₀₋₂—;
R₁₂ is independently selected from the group consisting of C₁-C₆ alkyl, hydroxyl, C₁-C₆ alkoxy, or fluoro, provided that when R₁₂ is hydroxy or fluoro, then R₁₂ is not bound to a carbon adjacent to a nitrogen; or two R₁₂ substituents form a C₁ to C₂ alkyl bridge from one ring carbon to another non-adjacent ring carbon; or R₁₂ is ═O;
R₁₃ is independently selected from the group consisting of C₁-C₆ alkyl, hydroxyl, C₁-C₆ alkoxy, or fluoro, provided that when R₁₃ is hydroxy or fluoro then R^{13′} is not bound to a carbon adjacent to a nitrogen; or two R₁₃ substituents form a C₁ to C₂ alkyl bridge from one ring carbon to another non-adjacent ring carbon; or R₁₃ is ═O;
R₂ is independently selected from the group consisting of hydrogen, C₁-C₆ alkyl, or aryl, wherein said aryl group is optionally substituted with from 1 to 3 groups independently selected from halogen, —CF₃, —OCF₃, hydroxyl, or methoxy; or when two R₂₀ groups are present, said two R₂₀ groups taken together with the nitrogen to which they are bound can form a five or six membered heterocyclic ring;
R₂₂ is C₁-C₆ alkyl, R₃₄-aryl or heterocycloalkyl;
R₂₄ is H, C₁-C₆ alkyl, —SO₂R₂ or R₃₄-aryl;
R₂₅ is independently selected from the group consisting of C₁-C₆ alkyl, halogen, —CN, —NO₂, —CF₃, —OH, C₁-C₆ alkoxy, (C₁-C₆)alkyl-C(O)—, aryl-C(O)—, —C(O)OR₂₉, —N(R₄)(R₅), N(R₄)(R₅)—C(O)—, N(R₄)(R₅)—S(O)₁₋₂—, R₂₂—S(O)₀₋₂—, halo-(C₁-C₆)alkyl- or halo-(C₁-C₆)alkoxy-(C₁-C₆)alkyl-;
R₂₉ is H, C₁-C₆ alkyl, C₃-C₆ cycloalkyl, R₃₅-aryl or R₃₅-aryl(C₁-C₆)alkyl-;
R₃₀ is H, C₁-C₆ alkyl-, R₃₅-aryl or R₃₅-aryl(C₁-C₆)alkyl-;
R₃₁ is H, C₁-C₆ alkyl-, R₃₅-aryl, R₃₅-aryl(C₁-C₆)alkyl-, R₃₅-heteroaryl, (C₁-C₆)alkyl-C(O)—, R₃₅-aryl-C(O)—, N(R₄)(R₅)—C(O)—, (C₁-C₆)alkyl-S(O)₂— or R₃₅-aryl-S(O)₂—;
or R₃₀ and R₃₁ together are —(CH₂)₄₋₅—, —CH₂)₂—O—(CH₂)₂— or —CH₂)₂—N(R₃₈)—(CH₂)₂— and form a ring with the nitrogen to which they are attached;
R₃₂ is 1 to 3 substituents independently selected from the group consisting of H, —OH, halogen, C₁-C₆ alkyl, C₁-C₆ alkoxy, R₃₅-aryl-O—, —SR₂₂, —CF₃, —OCF₃, —OCHF₂, —NR₃₉R₄₀, phenyl, R₃₃-phenyl, NO₂, —CO₂R₃₉, —CON(R₃₉)₂, —S(O)₂R₂₂, —S(O)₂N(R₂₀)₂, —N(R₂₄)S(O)₂R₂₂, —CN, hydroxy-(C₁-C₆)alkyl-, —OCH₂CH₂OR₂₂, and R₃₅-aryl(C₁-C₆)alkyl-O—, or two R₃₂ groups on adjacent carbon atoms together form a —OCH₂O— or —O(CH₂)₂O— group;
R₃₃ is 1 to 3 substituents independently selected from the group consisting of C₁-C₆ alkyl, halogen, —CN, —NO₂, —CF₃, —OCF₃, —OCHF₂ and —O—(C₁-C₆)alkyl;
R₃₄ is 1 to 3 substituents independently selected from the group consisting of H, halogen, —CF₃, —OCF₃, —OH and —OCH₃;
R₃₅ is 1 to 3 substituents independently selected from hydrogen, halo, C₁-C₆ alkyl, hydroxy, C₁-C₆ alkoxy, phenoxy, —CF₃, —N(R₃₆)₂, —COOR₂₀ and —NO₂;
R₃₆ is independently selected form the group consisting of H and C₁-C₆ alkyl;
R₃₇ is 1 to 3 substituents independently selected from hydrogen, halo, C₁-C₆ alkyl, hydroxy, C₁-C₆ alkoxy, phenoxy, —CF₃, —N(R₃₆)₂, —COOR₂₀, —C(O)N(R₂₉)₂ and —NO₂, or R₃₇ is one or two ═O groups;
R₃₈ is H, C₁-C₆ alkyl, R₃₅-aryl, R₃₅-aryl(C₁-C₆)alkyl-, (C₁-C₆)alkyl-SO₂ or halo(C₁-C₆)alkyl-SO₂—;
R₃₉ is independently selected from the group consisting of hydrogen, C₁-C₆ alkyl, C₃-C₆ cycloalkyl, (C₃-C₆)cycloalkyl(C₁-C₆)alkyl, R₃₃-aryl, R₃₃-aryl(C₁-C₆)alkyl, and R₃₂-heteroaryl; and
R₄₀ is hydrogen, C₁-C₆ alkyl, —C(O)R₂₀, —C(O)₂R₂₀, —C(O)N(R₂₀)₂, (C₁-C₆)alkyl-SO₂—, or (C₁-C₆)alkyl-SO₂—NH—;
or R₃₉ and R₄₀, together with the nitrogen to which they are attached, form an azetidinyl, pyrrolidinyl, piperidinyl, piperazinyl or morpholinyl ring.

24. The method according to claim 2, wherein the H₃ receptor antagonist/agonist is a compound of formula (XV) 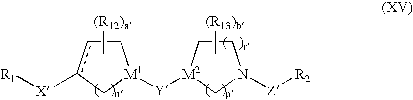
or a pharmaceutically acceptable salt or solvate thereof,
wherein, in formula (XV):
a′ is 0 to 3;
b′ is 0 to 3;
n′ is 1, 2 or 3;
p′ is 1, 2 or 3;
r′ is 0, 1, 2, or 3;
X′ is a bond or C₁-C₆ alkylene;
M¹ is CH or N;
M² is C(R₃) or N;
with the provisos that when M² is N, p′ is not 1; and that when r, is 0, M² is C(R₃); and that the sum of p′ and r′ is 1 to 4;
Y′ is —C(═O)—, —C(═S)—, —(CH₂)_{q′}—, —NR₄C(═O)—, —C(═O)NR₄—, —C(═O)CH₂—, —SO₁₋₂—, —C(═N—CN)—NH— or —NH—C(═N—CN)—; with the provisos that when M¹ is N, Y′ is not —NR₄C(═O)— or —NH—C(═N—CN)—; and when M² is N, Y′ is not —C(═O)NR₄— or —C(═N—CN)—NH—;
q′ is 1 to 5, provided that when M¹ and M² are both N, q′ is not 1;
Z′ is a bond, C₁-C₆ alkylene, C₂-C₆ alkenylene, —C(═O)—, —CH(CN)— or —CH₂C(═O)NR₄—;
R₁ is 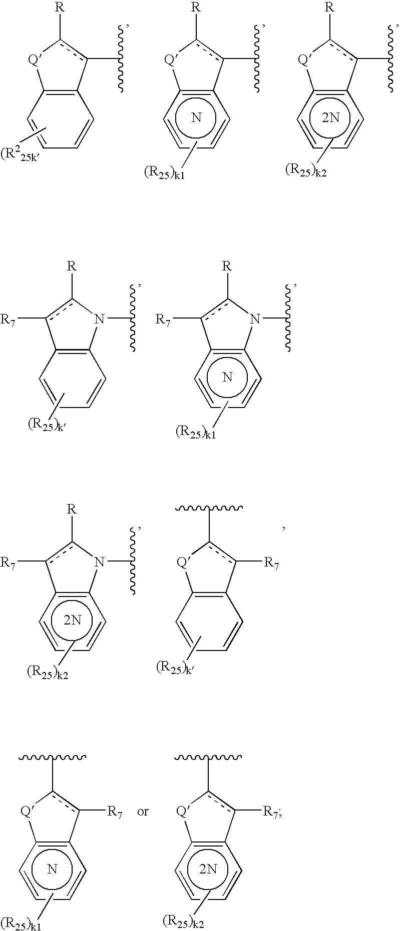
Q′ is —N(R₈)—, —S— or —O—;
k′ is 0, 1, 2, 3 or 4;
k1 is 0, 1, 2 or 3;
k2 is 0, 1 or 2;
the dotted line represents an optional double bond;
R and R₇ are independently selected from the group consisting of H, C₁-C₆ alkyl, halo(C₁-C₆)alkyl-, C₁-C₆ alkoxy, (C₁-C₆)alkoxy-(C₁-C₆)alkyl-, (C₁-C₆)-alkoxy-(C₁-C₆)alkoxy, (C₁-C₆)alkoxy-(C₁-C₆)alkyl-SO₀₋₂, R₃₂-aryl(C₁-C₆)alkoxy-, R₃₂-aryl-(C₁-C₆)alkyl-, R₃₂-aryl, R₃₂-aryloxy, R₃₂-heteroaryl, (C₃-C₆)cycloalkyl, (C₃-C₆)cycloalkyl-(C₁-C₆)alkyl, (C₃-C₆)cycloalkyl-(C₁-C₆)alkoxy, (C₃-C₆)cycloalkyl-oxy-, R₃₇-heterocyclo-alkyl, N(R₃₀)(R₃₁)—(C₁-C₆)alkyl-, —N(R₃₀)(R₃₁), —NH—(C₁-C₆)alkyl-O—(C₁-C₆)alkyl, —NHC(O)NH(R₂₉); R₂₉—S(O)₀₋₂—, halo(C₁-C₆)alkyl-S(O)₀₋₂—, N(R₃₀)(R₃₁)-(C₁-C₆)alkyl-S(O)₀₋₂—, benzoyl, (C₁-C₆)alkoxy-carbonyl, R₃₇-heterocycloalkyl-N(R₂₉)—C(O)—, (C₁-C₆)alkyl-N(R₂₉)—C(O)—, (C₁-C₆)alkyl-N(C₁-C₆ alkoxy)-C(O)— and —C(═NOR₃₆)R₃₆; and when the optional double bond is not present, R₇ can be OH;
R₈ is H, C₁-C₆ alkyl, halo(C₁-C₆)alkyl-, (C₁-C₆)alkoxy-(C₂-C₆)alkyl-, R₃₂-aryl(C₁-C₆)alkyl-, R₃₂-aryl, R₃₂-heteroaryl, R₃₂-heteroaryl(C₁-C₆)alkyl-, (C₃-C₆)cycloalkyl, (C₃-C₆)cycloalkyl-(C₁-C₆)alkyl, R₃₇-heterocycloalkyl, R₃₇-heterocycloalkyl(C₁-C₆)alkyl, N(R₃₀)(R₃₁)—(C₂-C₆)alkyl-, R₂₉—S(O)₂—, halo(C₁-C₆)alkyl-S(O)₂—, R₂₉—S(O)₀₋₁—(C₂-C₆)alkyl-, halo(C₁-C₆)alkyl-S(O)₀₋₁₀—(C₂-C₆)alkyl-, (C₁-C₆)alkyl-N(R₂₉)—SO₂—, or R₃₂-heteroaryl-SO₂;
R₂ is a six-membered heteroaryl ring having 1 or 2 heteroatoms independently selected from N or N—O, with the remaining ring atoms being carbon; a five-membered heteroaryl ring having 1, 2 or 3 heteroatoms independently selected from N, O or S, with the remaining ring atoms being carbon; R₃₂-quinolyl; R₃₂-aryl; 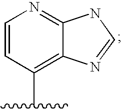
or heterocycloalkyl; wherein said six-membered heteroaryl ring or said five-membered heteroaryl ring is optionally substituted by R₆;
R₃ is H, halogen, C₁-C₆ alkyl, —OH or (C₁-C₆)alkoxy;
R₄ is independently selected from the group consisting of hydrogen, C₁-C₆ alkyl, C₃-C₆ cycloalkyl, (C₃-C₆)cycloalkyl(C₁-C₆)alkyl, R₃₃-aryl, R₃₃-aryl(C₁-C₆)alkyl, and R₃₃-heteroaryl;
R₅ is hydrogen, C₁-C₆ alkyl, —C(O)R₂₀, —C(O)₂R₂₀, —C(O)N(R₂₀)₂, R₃₃-aryl(C₁-C₆)alkyl or (C₁-C₆)alkyl-SO₂—;
R₆ is 1 to 3 substituents independently selected from the group consisting of —OH, halogen, C₁-C₆ alkyl, C₁-C₆ alkoxy, —CF₃, —NR₄R₅, —(C₁-C₆)alkyl-NR₄R₅, phenyl, R₃₃-phenyl, NO₂, —CO₂R₄, —CON(R₄)₂, —NHC(O)N(R₄)₂, R₃₂-heteroaryl-SO₂-NH—, R₃₂-aryl-(C₁-C₆)alkyl-NH—, R₃₂-heteroaryl-(C₁-C₆)alkyl-NH—, R₃₂-heteroaryl-NH—C(O)—NH— and R₃₇-heterocyclo-alkyl-N(R₂₉)—C(O)—;
R₁₂ is independently selected from the group consisting of C₁-C₆ alkyl, hydroxyl, C₁-C₆ alkoxy, or fluoro, provided that when R₁₂ is hydroxy or fluoro, then R₁₂ is not bound to a carbon adjacent to a nitrogen; or R₁₂ forms a C₁ to C₂ alkyl bridge from one ring carbon to another ring carbon;
R₁₃ is independently selected from the group consisting of C₁-C₆ alkyl, hydroxyl, C₁-C₆ alkoxy, or fluoro, provided that when R₁₃ is hydroxy or fluoro then R₁₃ is not bound to a carbon adjacent to a nitrogen; or forms a C₁ to C₂ alkyl bridge from one ring carbon to another ring carbon; or R₁₃ is ═O;
R₂₀ is independently selected from the group consisting of hydrogen, C₁-C₆ alkyl, or aryl, wherein said aryl group is optionally substituted with from 1 to 3 groups independently selected from halogen, —CF₃, —OCF₃, hydroxyl, or methoxy; or when two R₂₀ groups are present, said two R₂₀ groups taken together with the nitrogen to which they are bound can form a five or six membered heterocyclic ring;
R₂₂ is C₁-C₆ alkyl, R₃₄-aryl or heterocycloalkyl;
R₂₄ is H, C₁-C₆ alkyl, —SO₂R₂₂ or R₃₄-aryl;
R₂₅ is independently selected from the group consisting of C₁-C₆ alkyl, halogen, —CF₃, —OH, C₁-C₆ alkoxy, (C₁-C₆)alkyl-C(O)—, aryl-C(O)—, N(R₄)(R₅)—C(O)—, N(R₄)(R₅)—S(O)₁₋₂—, halo-(C₁-C₆)alkyl- or halo-(C₁-C₆)alkoxy-(C₁-C₆)alkyl-;
R₂₉ is H, C₁-C₆ alkyl, R₃₅-aryl or R₃₅-aryl(C₁-C₆)alkyl-;
R₃₀ is H, C₁-C₆ alkyl-, R₃₅-aryl or R₃₅-aryl(C₁-C₆)alkyl-;
R₃₁ is H, C₁-C₆ alkyl-, R₃₅-aryl, R₃₅-aryl(C₁-C₆)alkyl-, (C₁-C₆)alkyl-C(O)—, R₃₅-aryl-C(O)—, N(R₄)(R₅)—C(O)—, (C₁-C₆)alkyl-S(O)₂— or R₃₅-aryl-S(O)₂—;
or R₃₀ and R₃₁ together are —(CH₂)₄₋₅—, —CH₂)₂—O—(CH₂)₂— or —CH₂)₂—N(R₂₉)—(CH₂)₂— and form a ring with the nitrogen to which they are attached;
R₃₂ is 1 to 3 substituents independently selected from the group consisting of H, —OH, halogen, C₁-C₆ alkyl, C₁-C₆ alkoxy, R₃₅-aryl-O—, —SR₂₂, —CF₃, —OCF₃, —OCHF₂, —NR₄R₅, phenyl, R₃₃-phenyl, NO₂, —CO₂R₄, —CON(R₄)₂, —S(O)₂R₂₂, —S(O)₂N(R₂₀)₂, —N(R₂₄)S(O)₂R₂₂, —CN, hydroxy-(C₁-C₆)alkyl-, —OCH₂CH₂OR₂₂, and R₃₅-aryl(C₁-C₆)alkyl-O—, wherein said aryl group is optionally substituted with 1 to 3 independently selected halogens;
R₃₃ is 1 to 3 substituents independently selected from the group consisting of C₁-C₆ alkyl, halogen, —CN, —NO₂, —OCHF₂ and —O—(C₁-C₆)alkyl;
R₃₄ is 1 to 3 substituents independently selected from the group consisting of H, halogen, —CF₃, —OCF₃, —OH and —OCH₃.
R₃₅ is 1 to 3 substituents independently selected from the group consisting of hydrogen, halo, C₁-C₆ alkyl, hydroxy, C₁-C₆ alkoxy, phenoxy, —CF₃, —N(R₃₆)₂, —COOR₂₀ and —NO₂;
R₃₆ is independently selected from the group consisting of H and C₁-C₆ alkyl; and
R₃₇ is independently selected from the group consisting of H, C₁-C₆ alkyl and (C₁-C₆)alkoxycarbonyl.

25. The method according to claim 2, wherein the H₃ receptor antagonist/agonist is a compound of formula (XVI) 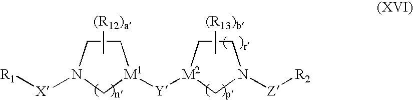
or a pharmaceutically acceptable salt or solvate thereof,
wherein, in formula (XVI):
(A) R₁ is selected from:
(1) aryl;
(2) heteroaryl;
(3) heterocycloalkyl
(4) alkyl;
(5) —C(O)N(R_{4B})₂;
(6) cycloalkyl;
(7) arylalkyl;
(8) heteroarylheteroaryl; or
(9) a group selected from: 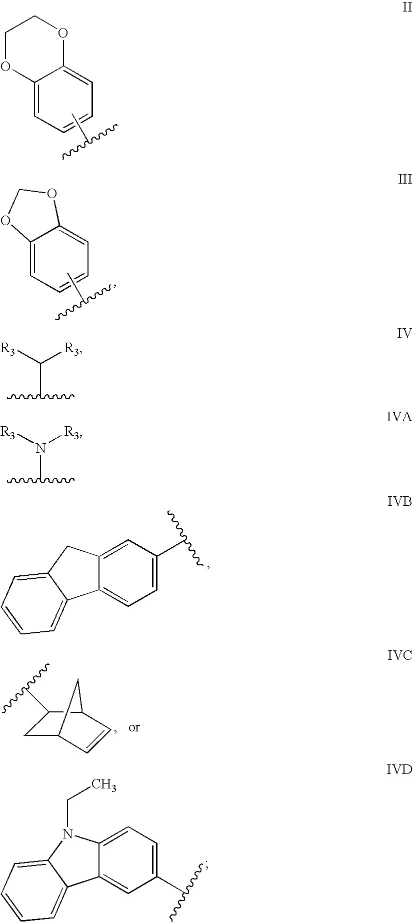
said aryl (see (A)(1) above), heteroaryl (see (A)(2) above), aryl portion of arylalkyl (see (A)(7) above), phenyl ring of formula II (see (A)(9) above), phenyl ring of formula III (see (A)(9) above), phenyl rings of formula IVB (see (A)(9) above), or phenyl rings of formula IVD (see (A)(9) above) are optionally substituted with 1 to 3 substituents independently selected from:
(1) halogen;
(2) hydroxyl;
(3) lower alkoxy;
(4) —Oaryl;
(5) —SR₂₂;
(6) —CF₃;
(7) —OCF₃;
(8) —OCHF₂;
(9) —NR₄R₅;
(10) phenyl;
(11) NO₂,
(12) —CO₂R₄;
(13) —CON(R₄)₂ wherein each R₄ is the same or different;
(14) —S(O)₂R₂₂;
(15) —S(O)₂N(R₂₀)₂ wherein each R₂₀ is the same or different;
(16) —N(R₂₄)S(O)₂R₂₂;
(17) —CN;
(18) —CH₂OH;
(19) —OCH₂CH₂OR₂₂;
(20) alkyl;
(21) substituted phenyl wherein said phenyl has 1 to 3 substituents independently selected from alkyl, halogen, —CN, —NO₂, —OCHF₂, —Oalkyl;
(22) —Oalkylaryl (preferably —Oalkylphenyl or —Oalkyl-substituted phenyl, e.g., —OCH₂dichlorophenyl, such as —CH₂-2,6-dichlorophenyl or —OCH₂-2-chloro-6-fluorophenyl) wherein said aryl group is optionally substituted with 1 to 3 independently selected halogens; or
(23) phenyl;
(B) X′ is selected from alkyl (e.g., —(CH₂)_{q′}— or branched alkyl) or —S(O)₂—;
(C) Y′ represents
(1) a single bond (i.e., Y′ represents a direct bond from M¹ to M²); or
(2) Y′ is selected from —C(O), —C(S)—, —(CH₂)_{q′}—, or —NR₄C(O)—; with the provisos that:
(a) when M¹ is N, then Y′ is not —NR₄C(O)—; and
(b) when Y′ is a bond, then M¹ and M² are both carbon;
(D) M¹ and M² are independently selected from C or N;
(E) Z′ is selected from: C₁-C₆ alkyl, —SO₂—, —C(O)— or —C(O)NR₄—;
(F) R₂ is selected from:
(1) a six-membered heteroaryl ring having 1 or 2 heteroatoms independently selected from N or N—O (i.e., N-oxide), with the remaining ring atoms being carbon;
(2) a five-membered heteroaryl ring having 1 to 3 heteroatoms selected from nitrogen, oxygen, or sulfur with the remaining ring atoms being carbon; or
(3) an alkyl group;
(4) an aryl group or an aryl group that is substituted with 1 to 3 substituents independently selected from: halogen, —Oalkyl, —OCF₃, —CF₃, —CN, —NO₂, —NHC(O)CH₃, or —O(CH₂)_{q′}N(R^{10A})₂;
(5) —N(R^{11A})₂ wherein each R^{11A} is independently selected from: H, alkyl or aryl;
(6) a group of the formula: 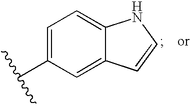
(7) a heteroarylheteroaryl group,?
said five membered heteroaryl ring ((F)(2) above) or six-membered heteroaryl ring ((F)(1) above) is optionally substituted with 1 to 3 substituents selected from:
(a) halogen;
(b) hydroxyl;
(c) lower alkyl;
(d) lower alkoxy;
(e) —CF₃;
(f) —NR₄R₅;
(g) phenyl;
(h) —NO₂;
(i) —C(O)N(R₄)₂ (wherein each R₄ is the same or different);
(j) —C(O)₂R₄; or
(k) phenyl substituted with 1 to 3 substituents independently selected from: halogen, —Oalkyl, —OCF₃, —CF₃, —CN, —NO₂ or —O(CH₂)_{q}N(R^{10A})₂;
(G) R₃ is selected from:
(1) aryl;
(2) heteroaryl;
(3) heterocycloalkyl
(4) alkyl; or
(5) cycloalkyl;
wherein said aryl or heteroaryl R₃ groups is optionally substituted with 1 to 3 substituents independently selected from:
(a) halogen;
(b) hydroxyl;
(c) lower alkoxy;
(d) —Oaryl;
(e) —SR₂₂;
(f) —CF₃;
(g) —OCF₃;
(h) —OCHF₂;
(i) —NR₄R₅;
(j) phenyl;
(k) —NO₂,
(l) —CO₂R₄;
(m) —CON(R₄)₂ wherein each R₄ is the same or different;
(n) —S(O)₂R₂₂;
(o) —S(O)₂N(R₂₀)₂ wherein each R₂₀ is the same or different;
(p) —N(R₂₄)S(O)₂R₂₂;
(q) —CN;
(r) —CH₂OH;
(s) —OCH₂CH₂OR₂₂; or
(t) alkyl;
(H) R₄ is selected from:
(1) hydrogen;
(2) C₁-C₆ alkyl;
(3) cycloalkyl;
(4) cycloalkylalkyl;
(5) heterocycloalkylalkyl;
(6) bridged bicyclic cycloalkyl ring;
(7) aryl having a fused heterocycloalkyl ring bound to said aryl ring;
(8) aryl;
(9) arylalkyl;
(10) alkylaryl;
(11) —(CH₂)_{d′}CH(R^{12A})₂ wherein d is 1 to 3, and each R_{12A} is independently selected from phenyl or substituted phenyl, said substituted phenyl being substituted with 1 to 3 substituents independently selected from: halogen, —Oalkyl, —OCF₃, —CF₃, —CN, or —NO₂;
(12) heterocycloalkylheteroaryl; or
(13) —(C₁ to C₆)alkylene-O—R₂₂;
wherein the aryl R₄ group, the aryl portion of the arylalkyl R₄ group, or the aryl portion of the alkylaryl R₄ group is optionally substituted with 1 to 3 substituents independently selected from:
(a) halogen;
(b) hydroxyl;
(c) lower alkyl;
(d) lower alkoxy;
(e) —CF₃;
(f) —N(R₂₀)(R₂₄),
(g) phenyl;
(h) —NO₂;
(i) —C(O)N(R₂₀)₂ (wherein each R₂₀ is the same or different),
(j) —C(O)R₂₂;
(i) —(CH₂)_{k′}-cycloalkyl;
(j) —(CH₂)_{q′}-aryl; or
(k) —(CH₂)_{m′}—OR₂₂;
(I) each R_{4B} is independently selected from: H, heteroaryl, alkyl, alkenyl, a group of the formula 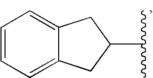
arylalkyl, or arylalkyl wherein the aryl moiety is substituted with 1-3 substituents independently selected from: halogen;
(J) R₅ is selected from: hydrogen, C₁-C₆ alkyl, —C(O)R₂₀, —C(O)₂R₂₀, —C(O)N(R₂₀)₂ (wherein each R₂₀ is the same or different);
(K) each R^{10A} is independently selected from H or C₁ to C₆ alkyl or each R_{10A}, taken together with the nitrogen atom to which they are bound, forms a 4 to 7 membered heterocycloalkyl ring;
(L) R₁₂ is
(1) selected from alkyl, hydroxyl, alkoxy, or fluoro, provided that when R₁₂ is hydroxy or fluoro then R₁₂ is not bound to a carbon adjacent to a nitrogen; or
(2) R₁₂ forms an alkyl bridge from one ring carbon to another ring carbon;
(M) R₁₃ is
(1) selected from alkyl, hydroxyl, alkoxy, or fluoro, provided that when R₁₃ is hydroxy or fluoro then R₁₃ is not bound to a carbon adjacent to a nitrogen; or
(2) R₁₃ forms an alkyl bridge from one ring carbon to another ring carbon;
(N) R₂₀ is selected from hydrogen, alkyl, or aryl, wherein said aryl group is optionally substituted with from 1 to 3 groups independently selected from: halogen, —CF₃, —OCF₃, hydroxyl, or methoxy; or when two R₂₀ groups are present, said two R₂₀ groups taken together with the nitrogen to which they are bound form a five or six membered heterocyclic ring;
(O) R₂₂ is selected from: heterocycloalkyl, alkyl or aryl, wherein said aryl group is optionally substituted with 1 to 3 groups independently selected from halogen, —CF₃, —OCF₃, hydroxyl, or methoxy;
(P) R₂₄ is selected from: hydrogen, alkyl, —SO₂R₂₂, or aryl, wherein said aryl group is optionally substituted with 1 to 3 groups independently selected from halogen, —CF₃, —OCF₃, hydroxyl, or methoxy;
(Q) a′ is 0 to 2;
(R) b′ is 0 to 2;
(S) k′ is 1 to 5;
(T) m′ is 2 to 5;
(U) n′ is 1, 2 or 3 with the proviso that when M¹ is N, then n′ is not 1;
(V) p′ is 1, 2 or 3 with the proviso that when M² is N, then p′ is not 1;
(W) q′ is 1 to 5; and
(X) r′ is 1, 2, or 3 with the proviso that when r′ is 2 or 3, then M² is C and ′p is 1.

26. The method according to claim 2, wherein the H₃ receptor antagonist/agonist is a compound of formula (XVII) 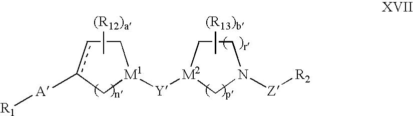
or a pharmaceutically acceptable salt or solvate thereof,
wherein, in formula XVII:
the dotted line represents an optional double bond;
a′ is 0 to 3;
b′ is 0 to 3;
n′ is 1, 2 or 3;
p′ is 1, 2 or 3;
r′ is 0, 1, 2, or 3;
with the provisos that when M² is N, p′ is not 1; and that when r′ is 0, M² is C; and that the sum of p′ and r′ is 1 to 4;
A′ is a bond or C₁-C₆ alkylene;
M¹ is CH or N;
M² is C(R₃) or N;
Y′ is —C(═O)—, —C(═S)—, —(CH₂)_{q′}—, —NR₄C(═O)—, —C(═O)NR₄—, —C(═O)CH₂—, —SO₁₋₂—, —NH—C(═N—CN)— or —C(═N—CN)—NH—; with the provisos that when M¹ is N, Y′ is not —NR₄C(═O)— or —NH—C(═N—CN)—; and when M² is N, Y′ is not —C(═O)NR₄— or —C(═N—CN)—NH—;
q′ is 1 to 5, provided that when M¹ and M² are both N, q′ is not 1;
Z′ is a bond, C₁-C₆ alkylene, C₁-C₆ alkenylene, —C(═O)—, —CH(CN)—, or —CH₂C(═O)NR₄—;
R₁ is 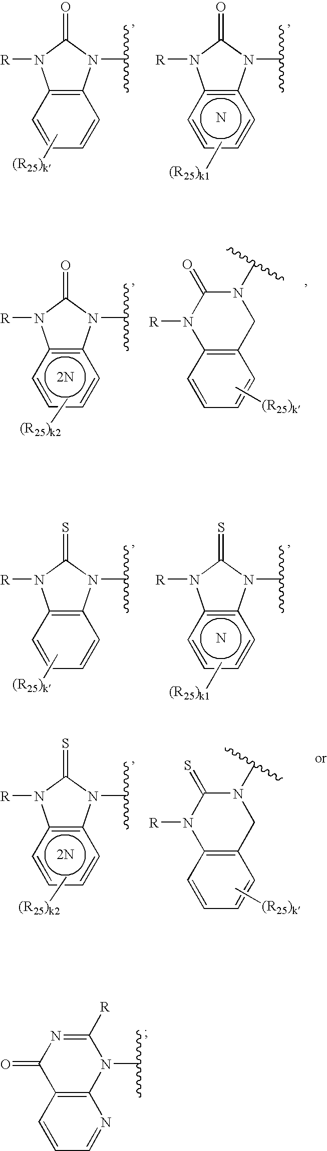
k′ is 0, 1, 2, 3 or 4;
k1 is 0, 1, 2 or 3;
k2 is 0, 1 or 2;
R is H, C₁-C₆ alkyl, hydroxy-(C₂-C₆)alkyl-, halo-(C₁-C₆)alkyl-, halo-(C₁-C₆)alkoxy-(C₁-C₆)alkyl-, R₂₉—O—C(O)—(C₁-C₆)alkyl-, (C₁-C₆)alkoxy-(C₁-C₆)alkyl-, N(R₃₀)(R₃₁)—(C₁-C₆)alkyl-, (C₁-C₆)alkoxy-(C₁-C₆)alkoxy-(C₁-C₆)alkyl-, R₃₂-aryl, R₃₂-aryl(C₁-C₆)alkyl-, R₃₂-aryloxy(C₁-C₆)alkyl-, R₃₂-heteroaryl, R₃₂-heteroaryl(C₁-C₆)alkyl-, (C₃-C₆)cycloalkyl, (C₃-C₆)cycloalkyl(C₁-C₆)alkyl-, N(R₃₀)(R₃₁)—C(O)—(C₁-C₆)alkyl-, or heterocycloalkyl(C₁-C₆)alkyl-;
R₂ is a six-membered heteroaryl ring having 1 or 2 heteroatoms independently selected from N or N—O, with the remaining ring atoms being carbon; a five-membered heteroaryl ring having 1, 2 or 3 heteroatoms independently selected is from N, O or S, with the remaining ring atoms being carbon; R₃₂-quinolyl; R₃₂-aryl; heterocycloalkyl; 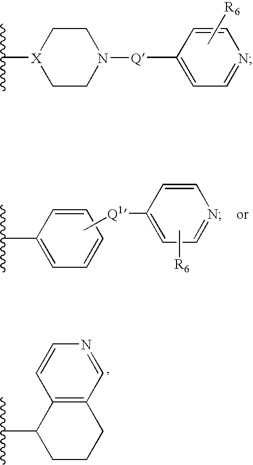
wherein said six-membered heteroaryl ring or said five-membered heteroaryl ring is optionally substituted by R₆;
X′ is C or N;
Q′ is a bond or C₁-C₆ alkylene;
Q^{1′} is a bond, C₁-C₆ alkylene or —N(R₄)—;
R₃ is H, halogen, C₁-C₆ alkyl, —OH or (C₁-C₆)alkoxy;
R₄ is independently selected from the group consisting of hydrogen, C₁-C₆ alkyl, C₃-C₆ cycloalkyl, (C₃-C₆)cycloalkyl(C₁-C₆)alkyl, R₃₃-aryl, R₃₃-aryl(C₁-C₆)alkyl, and R₃₂-heteroaryl;
R₅ is hydrogen, C₁-C₆ alkyl, —C(O)R₂₀, —C(O)₂R₂₀, —C(O)N(R₂₀)₂ or (C₁-C₆)alkyl-SO₂—;
R₆ is 1 to 3 substituents independently selected from the group consisting of —OH, halogen, C₁-C₆ alkyl-, C₁-C₆ alkoxy, C₁-C₆ alkylthio, —CF₃, —NR₄R₅, phenyl, R₃₃-phenyl, NO₂, —CO₂R₄, —CON(R₄)₂, 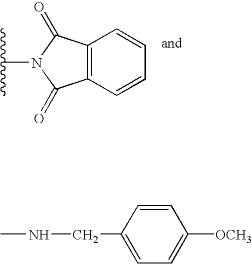
R₁₂ is independently selected from the group consisting of C₁-C₆ alkyl, hydroxyl, C₁-C₆ alkoxy, or fluoro, provided that when R₁₂ is hydroxy or fluoro, then R₁₂ is not bound to a carbon adjacent to a nitrogen; or R₁₂ forms a C₁ to C₂ alkyl bridge from one ring carbon to another ring carbon;
R₁₃ is independently selected from the group consisting of C₁-C₆ alkyl, hydroxyl, C₁-C₆ alkoxy, or fluoro, provided that when R₁₃ is hydroxy or fluoro then R₁₃ is not bound to a carbon adjacent to a nitrogen; or forms a C₁ to C₂ alkyl bridge from one ring carbon to another ring carbon; or R₁₃ is ═O;
R₂₀ is independently selected from the group consisting of hydrogen, C₁-C₆ alkyl, or aryl, wherein said aryl group is optionally substituted with from 1 to 3 groups independently selected from halogen, —CF₃, —OCF₃, hydroxyl, or methoxy; or when two R₂₀ groups are present, said two R₂₀ groups taken together with the nitrogen to which they are bound form a five or six membered heterocyclic ring;
R₂₂ is C₁-C₆ alkyl, R₃₄-aryl or heterocycloalkyl;
R₂₄ is H, C₁-C₆ alkyl, —SO₂R₂₂ or R₃₄-aryl;
R₂₅ is independently selected from the group-consisting of C₁-C₆ alkyl, halogen, —CF₃, —OH, C₁-C₆ alkoxy, (C₁-C₆)alkyl-C(O)—, aryl-C(O)—, N(R₄)(R₅)—C(O)—, N(R₄)(R₅)—S(O)₁₋₂—, halo-(C₁-C₆)alkyl- or halo-(C₁-C₆)alkoxy-(C₁-C₆)alkyl-;
R₂₉ is H, C₁-C₆ alkyl, R₃₅-aryl or R₃₅-aryl(C₁-C₆)alkyl-;
R₃₀ is H, C₁-C₆ alkyl-, R₃₅-aryl or R₃₅-aryl(C₁-C₆)alkyl-;
R₃, is H, C₁-C₆ alkyl-, R₃₅-aryl, R₃₅-aryl(C₁-C₆)alkyl-, (C₁-C₆)alkyl-C(O)—, R₃₅-aryl-C(O)—, N(R₄)(R₅)—C(O), (C₁-C₆)alkyl-S(O)₂— or R₃₅-aryl-S(O)₂—;
or R₃₀ and R₃₁ together are —(CH₂)₄₋₅—, —CH₂)₂—O—(CH₂)₂— or —CH₂)₂—N(R₂₉)—(CH₂)₂— and form a ring with the nitrogen to which they are attached;
R₃₂ is 1 to 3 substituents independently selected from the group consisting of H, —OH, halogen, C₁-C₆ alkyl, C₁-C₆ alkoxy, R₃₅-aryl-O—, —SR₂₂, —CF₃, —OCF₃, —OCHF₂, —NR₄R₅, phenyl, R₃₃-phenyl, NO₂, —CO₂R₄, —CON(R₄)₂, —S(O)₂R₂₂, —S(O)₂N(R₂₀)₂, —N(R₂₄)S(O)₂R₂₂, —CN, hydroxyl-(C₁-C₆)alkyl-, —OCH₂CH₂OR₂₂, and R₃₅-aryl(C₁-C₆)alkyl-O—, wherein said aryl group is optionally substituted with 1 to 3 independently selected halogens;
R₃₃ is 1 to 3 substituents independently selected from the group consisting of C₁-C₆ alkyl, halogen, —CN, —NO₂, —OCHF₂ and —O—(C₁-C₆)alkyl;
R₃₄ is 1 to 3 substituents independently selected from the group consisting of H, halogen, —CF₃, —OCF₃, —OH and —OCH₃.
R₃₅ is 1 to 3 substituents independently selected from hydrogen, halo, C₁-C₆ alkyl, hydroxyl, C₁-C₆ alkoxy, phenoxy, —CF₃, —N(R₃₆)₂, —COOR₂₀ and —NO₂; and
R₃₆ is independently selected form the group consisting of H and C₁-C₆ alkyl.

27. The method according to claim 1, wherein the cholesterol lowering agent inhibitor is a bile acid sequestrant.

28. The method according to claim 27, wherein the bile acid sequestrant is cholestyramine.

29. The method according to claim 1, wherein the cholesterol lowering agent is an HMG-CoA reductase inhibitor.

30. The method according to claim 29, wherein the HMG-CoA reductase inhibitor is selected from the group consisting of lovastatin, pravastatin, simvastatin, atorvastatin, fluvastatin, cerivastatin, CI-981, rivastatin, rosuvastatin and pitavastatin.

31. The method according to claim 1, wherein the cholesterol lowering agent is nicotinic acid (niacin) and/or a nicotinic acid agonist.

32. The method according to claim 1, wherein the cholesterol lowering agent is an activator of peroxisome proliferator-activated receptor.

33. The method according to claim 1 wherein the activator is a fibrate.

34. The method according to claim 33, where in the fibrate is clofibrate, gemfibrozil, ciprofibrate, bezafibrate, clinofibrate, binifibrate, lifibrol, or fenofibrate.

35. The method according to claim 2, which further comprises an effective amount of an HMG-CoA reductase inhibitor.

36. The method according to claim 18, wherein the HMG-CoA reductase inhibitor is selected from the group consisting of lovastatin, pravastatin fluvastatin, simvastatin, atorvastatin, cerivastatin, pitavastatin, and rosuvastatin.

37. The method according to claim 2, which further comprises as a third active component a PPAR activator, nicotinic acid and/or a nicotinic acid receptor agonist or a bile acid sequestrant.

38. The method according to claim 1, which further comprises an obesity control agent.

39. The method according to claim 2, which further comprises an obesity control agent.

40. The method according to claim 38, wherein the obesity control agent is selected from the group consisting of diethylpropion, mazindol, phenylpropanolamine, phentermine, phendimetrazine, phendamine tartrate, methamphetamine, phendimetrazine tartrate, sibutramine, fenfluramine, dexfenfluramine, fluoxetine, fluvoxamine paroxtine befloxatone, moclobemide, brofaromine, phenoxathine, esuprone, befol, toloxatone, pirlindol, amiflamine, sercloremine, bazinaprine, lazabemide, milacemide, caroxazone, and orlistat.

41. The method according to claim 40, which further comprises an HMG-CoA reductase inhibitor.

42. A method for the treatment, prevention or ameliorating the symptoms of nonalcoholic fatty liver disease (NAFLD) in a mammal in need thereof comprising the step of administering an effective amount of at least one sterol or 5-α-stanol absorption inhibitor or a pharmaceutically acceptable salt thereof or a solvate thereof.

43. A method for the treatment, prevention or ameliorating the symptoms of nonalcoholic fatty liver disease (NAFLD) in a mammal in need thereof comprising the step of administering an effective amount of at least one H₃ receptor antagonist/inverse agonist or a pharmaceutically acceptable salt thereof or a solvate thereof.

44. A method for the prevention or amelioration of the symptoms or the development of hepatic steatosis in a mammal in need there of comprising the step of administering an effective amount of antherapeutic composition comprising at least one cholesterol lowering agent and/or at least one H₃ receptor antagonist/inverse agonist to said mammal.

45. The method according to claim 44, which further comprises a HMG-CoA reductase inhibitor.

46. A method for the prevention or amelioration of the development of nonalcoholic steatohepatitis (NASH) in a mammal in need thereof by administering an effective amount of a therapeutic composition comprising at least one at least at least one H₃ receptor antagonist/inverse agonist and, optionally at least one cholesterol lowering agent to said mammal.

47. The method according to claim 46, which further comprises a HMG-CoA reductase inhibitor.

48. A method for the prevention or amelioration of the development of cirrhosis or heptacellular carcinoma in a mammal in need thereof comprising the step of administering an effective amount of a therapeutic composition comprising a at least one cholesterol lowering agent and/or at least one H₃ receptor antagonist/inverse agonist to said mammal.
